(19) United States
(12) Patent Application Publication
(10) Pub. No.: US 2024/0226312 A1
(43) Pub. Date: Jul. 11, 2024

(54) TREATMENT OF CANCER IN PATIENTS WITH SOLUBLE FR-ALPHA

(71) Applicant: ImmunoGen, Inc., Waltham, MA (US)

(72) Inventors: Eric WESTIN, Concord, MA (US); Jiuzhou WANG, Lexington, MA (US); Callum SLOSS, Wakefield, MA (US)

(73) Assignee: ImmunoGen, Inc., Waltham, MA (US)

(21) Appl. No.: 18/566,832

(22) PCT Filed: Jun. 2, 2022
(86) PCT No.: PCT/US2022/031927

§ 371 Date: Dec. 4, 2023

Related U.S. Application Data
(60) Provisional application No. 63/196,763, filed on Jun. 4, 2021.

(51) Int. Cl. A61K 47/68; A61K 39/00; A61K 47/65; A61P 35/00; C07K 16/28; G01N 33/574; G01N 33/68

(52) U.S. Cl. CPC A61K 47/68033; A61K 47/65; A61K 47/6849; A61P 35/00; C07K 16/28; G01N 33/57484; G01N 33/6848; A61K 2039/505; A61K 2039/545; G01N 2333/705; G01N 2474/20

(57) ABSTRACT

The present disclosure demonstrates that the amount of soluble folate receptor alpha (FRα) present in a cancer patient is a strong predictor of the efficacy of FRα-targeting therapies. Surprisingly, increased levels of soluble FRα are associated with improved outcomes. Accordingly, the present disclosure provides methods for treating cancer in patients with soluble FRα and methods for identifying a cancer as likely to respond to an anti-FRα therapy based on soluble FRα levels.

### REFERENCE TO RELATED APPLICATIONS

This application claims the priority benefit of U.S. Provisional Application No. 63/196,763, filed on Jun. 4, 2021, which is herein incorporated by reference in its entirety.

### REFERENCE TO A SEQUENCE LISTING SUBMITTED ELECTROTNICALLY VIA EFS-WEB

The content of the electronically submitted sequence listing (Name: 2921_110PC01_Seqlisting_ST25; Size: 97,063 bytes and Date of Creation: Jun. 1, 2022) is incorporated herein by reference in its entirety.

### FIELD OF THE DISCLOSURE

The field of this disclosure generally relates to methods of treating cancer in patients with soluble folate receptor alpha (FRα) and the use of anti-FRα active agents (e.g., antibodies and immunoconjugates) in such treatments.

### BACKGROUND OF THE DISCLOSURE

Cancer is one of the leading causes of death in the developed world, with over one million people diagnosed with cancer and 500,000 deaths per year in the United States alone. Overall it is estimated that more than 1 in 3 people will develop some form of cancer during their lifetime.

Folate receptor-α (FRα or FOLR1) is a glycosylphosphatidylinositol-linked cell-surface glycoprotein that has high affinity for folates. Its physiologic role in normal and cancerous tissues has not yet been fully elucidated. Most normal tissues do not express FRα, and transport of physiologic folates into most cells is thought to be mediated by several other proteins, most notably, reduced folate carrier. High levels of FRα have been found in serous and endometrioid epithelial ovarian cancer, endometrial adenocarcinoma, and non-small cell lung cancer of the adenocarcinoma subtype. Importantly, FRα expression is maintained in metastatic foci and recurrent carcinomas in ovarian cancer patients, and after chemotherapy in epithelial ovarian and endometrial cancers. These properties, together with the highly restricted expression of FRα on normal tissues, make FRα a highly promising target for targeted therapies such as ADCs.

Mirvetuximab soravtansine (IMGN853), a folate targeting antibody drug conjugate (ADC) that comprises a FRα targeting antibody conjugated to a potent tubulin-acting maytansinoid, DM4, was recently evaluated in the clinic in platinum-resistant ovarian cancer patients exhibiting medium and high membrane FRα levels as measured by immunohistochemistry (IHC) staining. The FORWARD I Phase 3 trial randomized 366 patients 2:1 to receive either mirvetuximab soravtansine or the physician's choice of single-agent chemotherapy (pegylated liposomal doxorubicin, topotecan, or weekly paclitaxel). While the trial did not meet its primary endpoint of improvement in progression-free survival (PFS) (in the overall population hazard ratio (HR)=0.98, p=0.897), the pre-specified high FRα sub-population (218/366) showed an overall response rate of 24% with IMGN853 treatment versus 10% for standard of care chemotherapy. In addition, in the pre-specified high FRα sub-population, the PFS was longer in patients who received IMGN853 compared with chemotherapy (HR=0.69, p-value=0.049), and overall survival was longer in patients who received IMGN853 compared with chemotherapy (HR=0.62, p-value=0.033). While, these results are encouraging for patients with tumors expressing high levels of membrane FRα as measured by IHC, a substantial portion of patients have tumors that do not have high levels of membrane FRα by IHC. Furthermore, soluble FRα could theoretically interfere with FRα-targeting therapies. Therefore, there is a need to understand the effect of soluble FRα on the safety and efficacy of FRα-targeting therapies and a need for methods to treat a broader patient population.

### SUMMARY OF THE DISCLOSURE

Provided herein are methods of treating cancer in a patient with soluble folate receptor alpha (α).

In some aspects, a method of treating cancer in a patient comprises administering a pharmaceutical composition comprising an anti-FRα active agent to a cancer patient with a soluble FRα level equal to or greater than a target soluble FRα level. In some aspects, the patient's soluble FRα has been detected in a sample obtained from the patient prior to the administration. In some aspects, a cancer sample obtained from the patient does not have a high FRα immunohistochemistry (IHC) score. In some aspects, a cancer sample obtained from the patient has a high FRα IHC score. In some aspects, no FRα IHC score has been obtained from the patient.

In some aspects, a method of treating cancer in a patient comprises (i) administering a pharmaceutical composition comprising an anti-FRα active agent to the patient if the patient has a soluble FRα level equal to or greater than a target soluble FRα level and/or a cancer sample obtained from the patient has a high FRα IHC score and (ii) administering chemotherapy to the patient if the patient does not have a soluble FRα level equal to or greater than a target soluble FRα level and a cancer sample obtained from the patient does not have a high FRα IHC score.

In some aspects, the method further comprises determining the level of soluble FRα in sample obtained from the patient prior to the administering of the active agent.

In some aspects, the method further comprises determining the FRα IHC score in a cancer sample obtained from the patient prior to the administration.

In some aspects, a method for identifying a cancer in a patient as likely to respond to an anti-FRα active agent comprises assaying for soluble FRα in a sample obtained from the patient and optionally determining the FRα IHC score in a tumor sample obtained from the patient, wherein the presence of a soluble FRα level equal to or greater than a target soluble FRα level and/or a high FRα IHC score indicates the cancer is likely to respond to the anti-FRα active agent. In some aspects, the method further comprises administering a pharmaceutical composition comprising the anti-FRα active agent to the patient if the cancer is likely to respond.

In some aspects, a level of soluble FRα that is not equal to or greater than a target soluble FRα level and an IHC score that is not high indicates the cancer is not likely to respond to a pharmaceutical composition comprising the anti-FRα active agent. In some aspects, a level of soluble FRα that is not equal to or greater than a target soluble FRα level and an IHC score that is not high indicates the cancer is likely to be more responsive to chemotherapy than to a pharmaceutical composition comprising the anti-FRα active agent.

In some aspects, the patient's level of soluble FRα is assessed using liquid chromatography-mass spectrometry (LC/MS), enzyme-linked immunosorbent assay (ELISA), and/or or meso scale discovery (MSD).

In some aspects, the target soluble FRα level is about 0.5 ng/mL. In some aspects, the target soluble FRα level is about 0.6 ng/mL. In some aspects, the target soluble FRα level is about 0.7 ng/mL. In some aspects, the target soluble FRα level is about 0.75 ng/mL. In some aspects, the target soluble FRα level is about 0.8 ng/mL. In some aspects, the target soluble FRα level is about 0.9 ng/mL. In some aspects, the target soluble FRα level is about 1 ng/mL. In some aspects, the target soluble FRα level is about 1.1 ng/mL. In some aspects, the target soluble FRα level is about 1.2 ng/mL. In some aspects, the target soluble FRα level is about 1.25 ng/mL. In some aspects, the target soluble FRα level is about 1.3 ng/mL. In some aspects, the target soluble FRα level is about 1.4 ng/mL. In some aspects, the target soluble FRα level is about 1.5 ng/mL. In some aspects, the target soluble FRα level is about 1.6 ng/mL. In some aspects, the target soluble FRα level is about 1.7 ng/mL. In some aspects, the target soluble FRα level is about 1.75 ng/mL. In some aspects, the target soluble FRα level is about 1.8 ng/mL. In some aspects, the target soluble FRα level is about 1.9 ng/mL. In some aspects, the target soluble FRα level is about 2.0 ng/mL. In some aspects, the target soluble FRα level is about 2.1 ng/mL. In some aspects, the target soluble FRα level is about 2.2 ng/mL. In some aspects, the target soluble FRα level is about 2.25 ng/mL. In some aspects, the target soluble FRα level is about 2.3 ng/mL. In some aspects, the target soluble FRα level is about 2.4 ng/mL. In some aspects, the target soluble FRα level is about 2.5 ng/mL. In some aspects, the target soluble FRα level is about 2.6 ng/mL. In some aspects, the target soluble FRα level is about 2.7 ng/mL. In some aspects, the target soluble FRα level is about 2.75 ng/mL. In some aspects, the target soluble FRα level is about 2.8 ng/mL. In some aspects, the target soluble FRα level is about 2.9 ng/mL. In some aspects, the target soluble FRα level is about 3.0 ng/mL. In some aspects, the target soluble FRα level is about 3.1 ng/mL. In some aspects, the target soluble FRα level is about 3.2 ng/mL. In some aspects, the target soluble FRα level is about 3.25 ng/mL. In some aspects, the target soluble FRα level is about 3.3 ng/mL. In some aspects, the target soluble FRα level is about 3.4 ng/mL. In some aspects, the target soluble FRα level is about 3.5 ng/mL. In some aspects, the target soluble FRα level is about 3.6 ng/mL. In some aspects, the target soluble FRα level is about 3.7 ng/mL. In some aspects, the target soluble FRα level is about 3.75 ng/mL. In some aspects, the target soluble FRα level is about 3.8 ng/mL. In some aspects, the target soluble FRα level is about 3.9 ng/mL. In some aspects, the target soluble FRα level is about 4.0 ng/mL. In some aspects, the target soluble FRα level is about 4.1 ng/mL. In some aspects, the target soluble FRα level is about 4.2 ng/mL. In some aspects, the target soluble FRα level is about 4.25 ng/mL. In some aspects, the target soluble FRα level is about 4.3 ng/mL. In some aspects, the target soluble FRα level is about 4.4 ng/mL. In some aspects, the target soluble FRα level is about 4.5 ng/mL. In some aspects, the target soluble FRα level is about 4.6 ng/mL. In some aspects, the target soluble FRα level is about 4.7 ng/mL. In some aspects, the target soluble FRα level is about 4.75 ng/mL. In some aspects, the target soluble FRα level is about 4.8 ng/mL. In some aspects, the target soluble FRα level is about 4.9 ng/mL. In some aspects, the target soluble FRα level is about 5 ng/mL.

In some aspects, the target soluble FRα level is the average soluble FRα level in patients with ovarian, primary peritonea, or fallopian tube cancer with a tumor with medium (50-74% cells positive) or high (at least 75% cells positive) membrane FRα levels as determined by percent staining (PS) 2+ staining intensity.

In some aspects, a high IHC score refers to at least 75% of tumor cells with percent staining (PS) 2+ staining intensity. In some aspects, a high IHC score refers to at least 50% of tumor cells with PS2+ staining intensity.

In some aspects, the cancer is a solid tumor. In some aspects, the cancer is selected from the group consisting of: ovarian cancer, uterine cancer, endometrial cancer, pancreatic cancer, renal cancer, lung cancer, peritoneal cancer, breast cancer, and fallopian tube cancer. In some aspects, the cancer is ovarian cancer. In some aspects, the ovarian cancer is platinum-resistant or platinum-refractory. In some aspects, the cancer is platinum-sensitive ovarian cancer. In some aspects, the ovarian cancer is epithelial ovarian cancer. In some aspects, the cancer is platinum-resistant, advanced high-grade epithelial ovarian cancer. In some aspects, the cancer is uterine cancer. In some aspects, the cancer is endometrial cancer. In some aspects, the cancer is pancreatic cancer. In some aspects, the cancer is renal cancer. In some aspects, the cancer is lung cancer. In some aspects, the lung cancer is non-small cell lung cancer. In some aspects, the cancer is peritoneal cancer. In some aspects, the peritoneal cancer is primary peritoneal cancer. In some aspects, the cancer is breast cancer. In some aspects, the breast cancer is triple negative breast cancer (TNBC). In some aspects, the cancer is fallopian tube cancer. In some aspects, the cancer is a recurrent cancer.

In some aspects, the active agent comprises an anti-FRα antibody or antigen-biding fragment thereof. In some aspects, the anti-FRα antibody or antigen-biding fragment thereof in the active agent binds to the same FRα epitope as an antibody comprising the VH of SEQ ID NO:38 and a VL of SEQ ID NO:44 and/or competitively inhibits binding of an antibody comprising the VH of SEQ ID NO:38 and a VL of SEQ ID NO:44 to FRα. In some aspects, the anti-FRα antibody or antigen-biding fragment thereof in the active agent comprises a variable heavy chain (VH) complementarity determining region (CDR) 1 comprising the amino acid sequence of SEQ ID NO:10, a VH-CDR2 comprising the amino acid sequence of SEQ ID NO:11, a VH-CDR3 comprising the amino acid sequence of SEQ ID NO:12, a variable light (VH)-CDR1 comprising the amino acid sequence of SEQ ID NO:15, a VL-CDR2 comprising the amino acid sequence of SEQ ID NO:16, and a VL-CDR3 comprising the amino acid sequence of SEQ ID NO:17. In some aspects, the anti-FRα antibody or antigen-biding fragment thereof in the active agent comprises a VH comprising the amino acid sequence of SEQ ID NO:38 and/or a VL comprising the amino acid sequence of SEQ ID NO:44. In some aspects, the anti-FRα antibody or antigen-biding fragment thereof in the active agent comprises a heavy chain comprising the amino acid sequence of SEQ ID NO:50 and/or a light chain comprising the amino acid sequence of SEQ ID NO:56. In some aspects, the anti-FRα antibody or antigen-biding fragment thereof in the active agent comprises a heavy chain comprising the same amino acid sequence as the amino acid sequence of the heavy chain encoded by the plasmid deposited with the American Type Culture Collection (ATCC) as PTA-10772 and/or a light chain comprising the same amino acid sequence as the amino acid sequence of the light chain encoded by the plasmid deposited with the ATCC as PTA-10774.

In some aspects, the anti-FRα antibody or antigen-biding fragment thereof in the active agent binds to the same FRα epitope as an antibody comprising the VH of SEQ ID NO:37 and a VL of SEQ ID NO:43 and/or competitively inhibits binding of an antibody comprising the VH of SEQ ID NO:37 and a VL of SEQ ID NO:43 to FRα. In some aspects, the anti-FRα antibody or antigen-biding fragment thereof in the active agent comprises a variable heavy chain (VH) complementarity determining region (CDR) 1 comprising the amino acid sequence of SEQ ID NO:2, a VH-CDR2 comprising the amino acid sequence of SEQ ID NO:3, a VH-CDR3 comprising the amino acid sequence of SEQ ID NO:4, a variable light (VH)-CDR1 comprising the amino acid sequence of SEQ ID NO:7, a VL-CDR2 comprising the amino acid sequence of SEQ ID NO:8, and a VL-CDR3 comprising the amino acid sequence of SEQ ID NO:9. In some aspects, the anti-FRα antibody or antigen-biding fragment thereof in the active agent comprises a VH comprising the amino acid sequence of SEQ ID NO:37 and/or a VL comprising the amino acid sequence of SEQ ID NO:43. In some aspects, the anti-FRα antibody or antigen-biding fragment thereof in the active agent comprises a heavy chain comprising the amino acid sequence of SEQ ID NO:49 and/or a light chain comprising the amino acid sequence of SEQ ID NO:55.

In some aspects, the active agent comprises an antigen-biding fragment of an anti-FRα antibody. In some aspects, the antigen-binding fragment is a single-chain variable fragment (scFv). In some aspects, the scFV comprises the amino acid sequence of SEQ ID NO:60.

In some aspects, the anti-FRα antibody or antigen-biding fragment thereof in the active agent is a monospecific antibody or antigen-binding fragment thereof. In some aspects, the anti-FRα antibody or antigen-biding fragment thereof in the active agent is a biparatopic antibody or antigen-binding fragment thereof. In some aspects, the biparatopic antibody or antigen-binding fragment thereof comprises the amino acid sequences of SEQ ID NOs:61, 62, and 56.

In some aspects, the active agent is an immunoconjugate comprising an anti-FRα antibody or antigen-biding fragment thereof conjugated to a cytotoxic agent. In some aspects, the cytotoxic agent is conjugated to the anti-FRα antibody or antigen-biding fragment thereof by a linker. In some aspects, the linker is selected from the group consisting of a cleavable linker, a non-cleavable linker, a hydrophilic linker, and a dicarboxylic acid based linker. In some aspects, the linker is selected from the group consisting of N-(γmaleimidobutryloxy)sulfosuccinimide ester (sulfo-GMBS or sGMBS), γmaleimidobutyric acid N-succinimidyl ester (GMBS), N-succinimidyl 4-(2-pyridyldithio)-2-sulfobutanoate (sulfo-SPDB); N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB), N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol] ester (NHS-PEG4-maleimide). In some aspects, the linker is sulfo-SPDB. In some aspects, the linker is sulfo-GMBS. In some aspects, the linker is GMBS.

In some aspects, the cytotoxic agent is selected from the group consisting of a maytansinoid, maytansinoid analog, benzodiazepine, taxoid, CC-1065, CC-1065 analog, duocarmycin, duocarmycin analog, calicheamicin, dolastatin, dolastatin analog, auristatin, tomaymycin derivative, and leptomycin derivative or a prodrug of the agent. In some aspects, the cytotoxic agent is a maytansinoid. In some aspects, the maytansinoid is DM4. In some aspects, the maytansinoid is DM21.

In some aspects, the immunoconjugate comprises 1 to 20 cytotoxic agents. In some aspects, the immunoconjugate comprises 1 to 10 cytotoxic agents. In some aspects, the immunoconjugate is represented by the following formula:

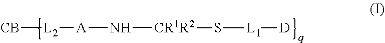

or a pharmaceutically acceptable salt thereof, wherein:
CB is an anti-FRα antibody or antigen-biding fragment thereof,
L₂ is represented by one of the following formula:

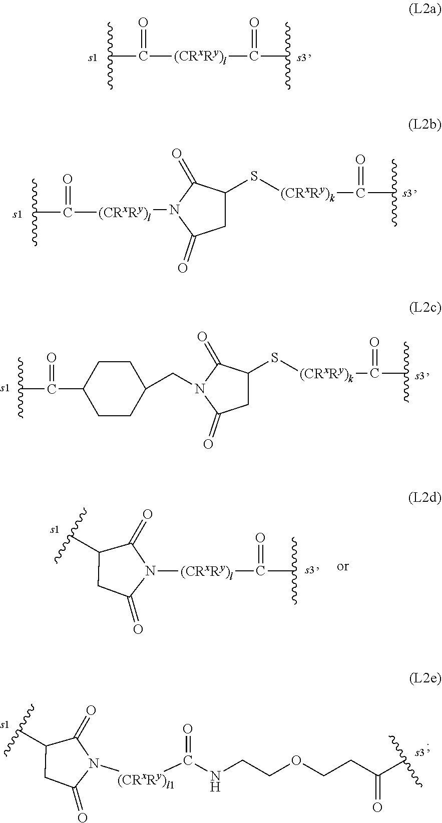

wherein:
R^{x}, R^{y}, R^{x′} and R^{y′}, for each occurrence, are independently H, —OH, halogen, —O—(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;
l1 is an integer from 2 to 5;
k1 is an integer from 1 to 5; and
s1 indicates the site connected to the cell-binding agent CB and s3 indicates the site connected to the A group;
A is an amino acid residue or a peptide comprising 2 to 20 amino acid residues;
R¹ and R² are each independently H or a C₁₋₃ alkyl;
L₁ is represented by the following formula:

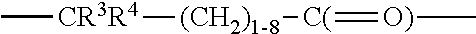

wherein R³ and R⁴ are each independently H or Me, and the —C(═O)— moiety in L₁ is connected to D;
D is represented by the following formula:

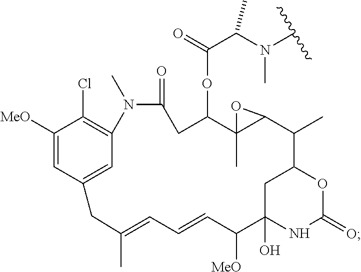 [SMILES: *N(C)[C@@H](C)C(=O)OC1CC(=O)N(C)c2cc(cc(OC)c2Cl)C/C(C)=C/C=C/C(OC)C2(O)CC(OC(=O)N2)C(C)C2OC12C]

q is an integer from 1 to 20.

In some aspects, R^{x}, R^{y}, R^{x′} and R^{y′} are all H; and l and k are each independently an integer an integer from 2 to 6. In some aspects, A is a peptide containing 2 to 5 amino acid residues. In some aspects, A is selected from the group consisting of Gly-Gly-Gly, Ala-Val, Val-Ala, D-Val-Ala, Val-Cit, D-Val-Cit, Val-Lys, Phe-Lys, Lys-Lys, Ala-Lys, Phe-Cit, Leu-Cit, Ile-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Ala-Ala, D-Ala-Ala-Ala, Ala-D-Ala-Ala, Ala-Ala-D-Ala, Ala-Leu-Ala-Leu (SEQ ID NO:54), P-Ala-Leu-Ala-Leu (SEQ ID NO:55), Gly-Phe-Leu-Gly (SEQ ID NO:56), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Gln-Val, Asn-Ala, Gln-Phe, Gln-Ala, D-Ala-Pro, and D-Ala-tBu-Gly, wherein the first amino acid in each peptide is connected to L₂ group and the last amino acid in each peptide is connected to —NH—CR¹R²—S-L₁-D. In some aspects, R¹ and R² are both H. In some aspects, L₁ is —(CH₂)₄₋₆—C(═O)—. In some aspects, D is represented by the following formula:

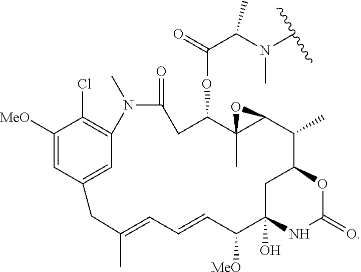 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some aspects, the immunoconjugate comprises an anti-FRα antibody or antigen-binding fragment thereof comprising a VH comprising the amino acid sequence of SEQ ID NO:38 and a VL comprising the amino acid sequence of SEQ ID NO:44, wherein the antibody or antigen-binding fragment thereof is conjugated to DM4 via a sulfo-SPDB linker. In some aspects, the immunoconjugate comprises an anti-FRα antibody comprising the a heavy chain comprising the amino acid sequence of SEQ ID NO:50 and a light chain comprising the amino acid sequence of SEQ ID NO:56. In some aspects, the pharmaceutical composition comprising an anti-folate receptor α (FRα) active agent comprises IMGN853. In some aspects, the pharmaceutical composition comprising IMGN853 is administered at a dose based on adjusted ideal body weight (AIBW), e.g., at a dose of about 6 mg/kg AIBW or at a dose of about 5 mg/kg AIBW.

In some aspects, the immunoconjugate is represented by the following formula:

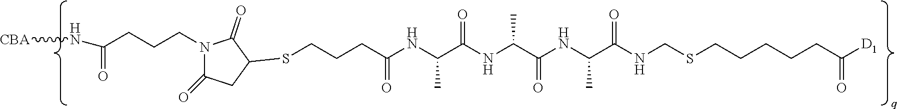

or a pharmaceutically acceptable salt thereof, wherein:
   CBA is an antibody or an antigen-binding fragment comprising the amino acid sequences of SEQ ID NOs:61, 62, and 56;
   D₁ is represented by the following formula:

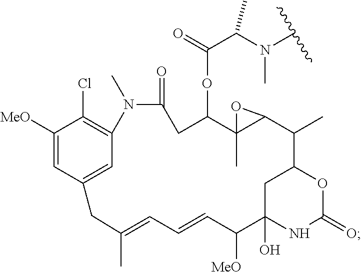 [SMILES: *N(C)[C@@H](C)C(=O)OC1CC(=O)N(C)c2cc(cc(OC)c2Cl)C/C(C)=C/C=C/C(OC)C2(O)CC(OC(=O)N2)C(C)C2OC12C]

q is an integer from 1 to 10.

In some aspects, the pharmaceutical composition comprising an anti-folate receptor α (FRα) active agent comprises IMGN151.

In some aspects, the pharmaceutical composition comprises anti-FRα immunoconjugates comprising an average of 2 to 5 cytotoxic agents per antibody or antigen-binding fragment thereof. In some aspects, the anti-FRα immunoconjugates comprise an average of 3 to 4 cytotoxic agents per antibody or antigen-binding fragment thereof. In some aspects, the pharmaceutical composition comprises anti-FRα immunoconjugates comprising an average of 3.5 cytotoxic agents per antibody or antigen-binding fragment thereof.

In some aspects, soluble FRα is detected using a detection antibody or antigen-binding fragment thereof that specifically binds to FRα, wherein an antibody comprising a VH comprising the amino acid sequence of SEQ ID NO:38 and a VL comprising the amino acid sequence of SEQ ID NO:44 does not competitively inhibit binding of the detection antibody or antigen-binding fragment thereof to FRα. In some aspects, soluble FRα is detected using a detection antibody or antigen-binding fragment thereof that specifically binds to FRα, wherein folic acid does not competitively inhibit binding of the detection antibody or antigen-binding fragment thereof to FRα. In some aspects, soluble FRα is detected using a detection antibody or antigen-binding fragment thereof that specifically binds to FRα and comprises the VH-CDR1, VH-CDR2, VH-CDR3, VL-CDR1, VL-CDR2 and VL-CDR3 amino acid sequences of: (a) SEQ ID NOs:18-20 and SEQ ID NOs:21-23, respectively; or (b) SEQ ID NOs:24-26 and SEQ ID NOs:27-29, respectively. In some aspects, the detection antibody or antigen-binding fragment thereof comprises the VH and VL amino acid sequences of (a) SEQ ID NO:39 and SEQ ID NO:45, respectively; or (b) SEQ ID NO:40 and SEQ ID NO:46, respectively. In some aspects, the detection antibody or antigen-binding fragment thereof comprises the heavy chain and light chain amino acid sequences of: (a) SEQ ID NO:51 and SEQ ID NO:57, respectively; or (b) SEQ ID NO:52 and SEQ ID NO:58, respectively.

In some aspects, soluble FRα is detected by (i) capturing FRα with an immunocapture reagent bound to a solid support (ii) eluting FRα from the solid support, (iii) digesting the eluted FRα, and (iv) performing LC/MS analysis on the digested FRα, wherein said FRα is detected by monitoring the chromatographic separation and mass spectrometric response of at least one signature FRα peptide. In some aspects, the solid support comprises a mass spectrometric immunoassay (MSIA) microcolumn. In some aspects, the solid support comprises magnetic beads. In some aspects, at least one wash step is performed prior to eluting FRα from the solid support. In some aspects, two or more wash steps are performed prior to eluting FRα from the solid support. In some aspects, the wash step comprises contacting the FRα bound to the solid support with washing buffers, a salt solution, and a detergent. In some aspects, FRα is eluted from the solid support with an acidic solution. In some aspects, the FRα is reduced and alkylated prior to digesting the FRα. In some aspects, the FRα is digested with Trypsin/Lys-C. In some aspects, digesting the FRα produces a peptide comprising the sequence of SEQ ID NO:68. In some aspects, digesting the FRα produces a peptide comprising the sequence of SEQ ID NO:69. In some aspects, digesting the FRα produces a peptide comprising the sequence of SEQ ID NO:70. In some aspects, digesting the FRα produces a peptide comprising the sequence of SEQ ID NO:71. In some aspects, at least two, at least three, or at least four signature peptides of FRα are selected and monitored at the LC/MS analysis step. In some aspects, the signature peptides comprise: (a) a peptide comprising the sequence of SEQ ID NO:68; (b) a peptide comprising the sequence of SEQ ID NO:69; (c) a peptide comprising the sequence of SEQ ID NO:70; and (d) a peptide comprising the sequence of SEQ ID NO:71.

In some aspects, the patient's level of soluble FRα is assessed using enzyme-linked immunosorbent assay (ELISA). In some aspects, the soluble FRα is detected in a body fluid sample. In some aspects, the body fluid is plasma, serum, or ascites fluid. In some aspects, the soluble FRα is detected in a peripheral blood sample.

In some aspects, the FRα IHC score is obtained using IHC that distinguishes between staining intensity and staining uniformity in a tumor sample as compared to a reference sample. In some aspects, the FRα IHC score is obtained using an IHC antibody or antigen-binding fragment thereof that specifically binds to FRα and comprises the VH-CDR1, VH-CDR2, VH-CDR3, VL-CDR1, VL-CDR2 and VL-CDR3 amino acid sequences of: SEQ ID NOs:30-32 and SEQ ID NOs:33-35, respectively. In some aspects, the IHC detection antibody or antigen-binding fragment thereof comprises the VH and VL amino acid sequences of: SEQ ID NO:41 and SEQ ID NO:47, respectively. In some aspects, the detection antibody or antigen-binding fragment thereof comprises the heavy chain and light chain amino acid sequences of: SEQ ID NO:53 and SEQ ID NO 59, respectively.

Also provided herein are pharmaceutical compositions for treating cancer in a patient with a soluble FRα level equal to or greater than a target soluble FRα according to any provided herein, wherein the composition comprises an anti-folate receptor α (FRα) active agent.

### BRIEF DESCRIPTIONS OF THE DRAWINGS

FIG. 1 shows the levels of soluble FRα detected in patients enrolled in a Phase 3 clinical trial comparing the safety and efficacy of IMGN853 with that of selected single-agent chemotherapy using either the original scale (left graph) or log 2 scale (right). (See Example 1.)

FIG. 2 shows the overall efficacy in IMGN853-treated subjects by soluble FRα level. PFS=progression free survival; OS=overall survival; ORR=objective response rate; BIRC=blinded independent review committee; and INV=investigators. (See Example 1.)

FIG. 3 is a progression free survival (PFS) hazard ratio forest plot showing the relative efficacy of IMGN853 and chemotherapy in patients with various soluble FRα levels. (See Example 1.)

FIG. 4 shows the proportion of patients with low, medium, and high membrane FRα as measured by IHC in each quartile (Q1-Q4) of soluble FRα levels (See Example 2.)

FIG. 5 shows 1+, 2+, and 3+ levels of membrane FRα immunohistochemistry (IHC) staining.

FIG. 6A shows soluble FRα distributions observed in MIRASOL and FORWARD I clinical trials. The horizontal lines that cut the boxes in half represent the median values, and the top of the boxes represent the 75% values. (See Example 3.)

FIG. 6B shows soluble FRα distributions observed in MIRASOL, FORWARD I, and SORAYA clinical trials. The horizontal lines that cut the boxes in half represent the median values, and the top of the boxes represent the 75% values. (See Example 3.)

FIGS. 7A and 7B show soluble FRα scores in patients with various IHC FRα PS2 scores. The horizontal lines that cut the boxes in half represent the median values, and the top of the boxes represent the 75% values (See Example 3.)

FIGS. 8A and 8B show surface tumor FRα expression across a soluble FRα distribution. (See Example 3.)

FIG. 9A provides pie charts showing the percentage of patients with low (Q1 and Q2) or high (Q3 and Q4) soluble FRα in patients with different FRα IHC (top row) and the percentage of patients with low (PS2<50), medium (PS2 50-74), or high (PS2≥75) FRα IHC in patients with different soluble FRα levels (bottom row). (See Example 2.)

FIG. 9B provides pie charts showing the percentage of patients with low (Q1 and Q2; <0.8 ng/mL) or high (Q3 and Q4; ≥0.8 ng/mL) soluble FRα in patients with different FRα IHC (top row) and the percentage of patients with PS2 0-24, PS2 25-49, PS250-74, or PS2≥75 FRα IHC in patients with different soluble FRα levels (bottom row). Soluble FRα quartiles were defined based on data from 440 MIRASOL patients with PS2 scores. Only the 404 patients with PS2 scores are included in the results shown in the figure. (See Example 2.)

FIG. 10 shows the correlation of liquid chromatography-mass spectrometry (LC-MS) and ELISA methods in detecting soluble FRα. (See Example 4.)

FIG. 11 shows that elevated soluble FRα levels as measured by ELISA correlate with increased progression free survival (PFS) and overall response rate (ORR). (See Example 4.)

### DETAILED DESCRIPTION OF THE DISCLOSURE

### I. Definitions

To facilitate an understanding of the present disclosure, a number of terms and phrases are defined below.

The terms “folate receptor 1,” “FRα,” “folate receptor alpha (FR-α),” or “FOLR1” as used herein, refers to any native FRα polypeptide, unless otherwise indicated. The terms encompasses “full-length,” unprocessed FRα polypeptide as well as any form of FRα polypeptide that results from processing within the cell. The term also encompasses naturally occurring variants of FRα, e.g., those encoded by splice variants and allelic variants. The FRα polypeptides described herein can be isolated from a variety of sources, such as from human tissue types or from another source, or prepared by recombinant or synthetic methods. Where specifically indicated, “FRα” can be used to refer to a nucleic acid that encodes a FRα polypeptide. Human FRα sequences are known and include, for example, the sequences publicly available at UniProtKB Accession No. P15328 (including isoforms). FRα can include a signal peptide (amino acids 1-24), the FRα protein chain (amino acids 25-234), and a propeptide that can be cleaved (amino acids 235 to 257). As used herein, the term “full-length human FRα” refers to polypeptide comprising the amino acid sequence SEQ ID NO:1, and the term “mature human FRα” refers to a polypeptide comprising amino acids 25-234 of SEQ ID NO:1).

(SEQ ID NO: 1)
MAQRMTTQLLLLLVWVAVVGEAQTRIAWARTELLNVCMNAKHHKEKPGP
EDKLHEQCRPWRKNACCSTNTSQEAHKDVSYLYRFNWNHCGEMAPACKR
HFIQDTCLYECSPNLGPWIQQVDQSWRKERVLNVPLCKEDCEQWWEDCR
TSYTCKSNWHKGWNWTSGFNKCAVGAACQPFHFYFPTPTVLCNEIWTHS
YKVSNYSRGSGRCIQMWFDPAQGNPNEEVARFYAAAMSGAGPWAAWPFL
LSLALMLLWLLS.

The term “soluble FRα” or “sFRC” as used herein refers to FRα protein that is soluble and that is not cell-associated. It is outside of the tumor tissue (e.g., in blood and/or plasma). In some aspects it includes the full-length FRα and the glycosylphosphatidyl inositol (GPI) anchor of FRα. In some aspects, soluble FRα includes only the full-length FRα. In some aspects the ECD and the GPI anchor can be embedded in a membrane (e.g., a soluble lipid raft). In some aspects, the soluble FRα comprises amino acids 1-233 of SEQ ID NO:1, amino acids 25-234 of SEQ ID NO:1, or a fragment thereof.

The term “anti-FRα antibody” or “an antibody that binds to FRα” refers to an antibody that is capable of binding FRα with sufficient affinity such that the antibody is useful as a diagnostic and/or therapeutic agent in targeting FRα. As used herein, such antibodies include, for example, monospecific and bispecific (e.g., biparatopic) antibodies. Unless otherwise specified, the extent of binding of an anti-FRα antibody to an unrelated, non-FRα protein is less than about 10% of the binding of the antibody to FRα as measured, e.g., by a radioimmunoassay (RIA). Examples of FRα antibodies are known in the art and are disclosed in U.S. Published Application Nos. 2012/0009181 and 2012/0282175 and U.S. Pat. No. 9,200,073 B2, and PCT publication WO 2011/106528 A1, and U.S. Published Application No. 2020/0362029, each of which is herein incorporated by reference in its entirety.

The term “IMGN853” (also known as “mirvetuximab soravtansine”) refers to a composition comprising immunoconjugates containing the huMov19 (or M9346A) antibody, the sulfo-SPDB linker, and the DM4 maytansinoid with an average drug to antibody ratio (DAR) of 3.5. The “huMov19” (or “M9346A”) antibody is an anti-FRα antibody comprising the full length heavy chain of SEQ ID NO:50 (comprising the variable heavy chain sequence SEQ ID NO:38, which is underlined in the context of SEQ ID NO:50 below) and the full length light chain of SEQ ID NO:56 (comprising the variable light chain sequence SEQ ID NO:44, which is underlined in the context of SEQ ID NO:56 below).

(SEQ ID NO: 50)
QVQLVQSGAEVVKPGASVKISCKASGYTFTGYFMNWVKQSPGQSLEWIG
RIHPYDGDTFYNQKFQGKATLTVDKSSNTAHMELLSLTSEDFAVYYCTR
YDGSRAMDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPG
(SEQ ID NO: 56)
DIVLTQSPLSLAVSLGQPAIISCKASQSVSFAGTSLMHWYHQKPGQQPR
LLIYRASNLEAGVPDRFSGSGSKTDFTLTISPVEAEDAATYYCQQSREY
PYTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

The huMov19 (M9346A) antibody is encoded by the plasmids deposited with the American Type Culture Collection (ATCC), located at 10801 University Boulevard, Manassas, VA 20110 on Apr. 7, 2010 under the terms of the Budapest Treaty and having ATCC deposit nos. PTA-10772 and PTA-10774. DM4 refers to N2′-deacetyl-N2′-(4-mercapto-4-methyl-1-oxopentyl) maytansinoid. “Sulfo-SPDB” refers to the N-succinimidyl 4-(2-pyridyldithio)-2-sulfobutanoate) linker.

The term “IMGN151” refers to a composition comprising immunoconjugates containing the KIH-FR57scfv-huMov19 antibody, the GMBS linker, and the DM21L maytansinoid with an average drug to antibody ratio (DAR) of 3.5. The sequences of the KIH-FR57scfv-huMov19 antibody are provided herein (see e.g., the deposits below and Table 8).

The KIH-FR57scfv-huMov19 antibody is encoded by the plasmids deposited with the American Type Culture Collection (ATCC) and having ATCC deposit nos. PTA-10774 (deposited in Apr. 7, 2010), PTA-125915 (“Mov19-Fc-hole”; deposited to the ATCC on Apr. 29, 2019 and received by the ATCC on Apr. 30, 2019), and PTA-125916 (“FR57scFv2-Fc-knob”; deposited to the ATCC on Apr. 29, 2019 and received by the ATCC on Apr. 30, 2019).

The terms “elevated” FRα, “increased expression” of FRα, or “overexpression” of FRα in a particular tumor, tissue, or sample refers to FRα (a FRα polypeptide or a nucleic acid encoding such a polypeptide) that is present at a level higher than that which is present in a healthy or non-diseased (native, wild type) tissue, cells, or samples of the same type or origin. Such increased expression or overexpression can be caused, for example, by mutation, gene amplification, increased transcription, increased translation, or increased protein stability.

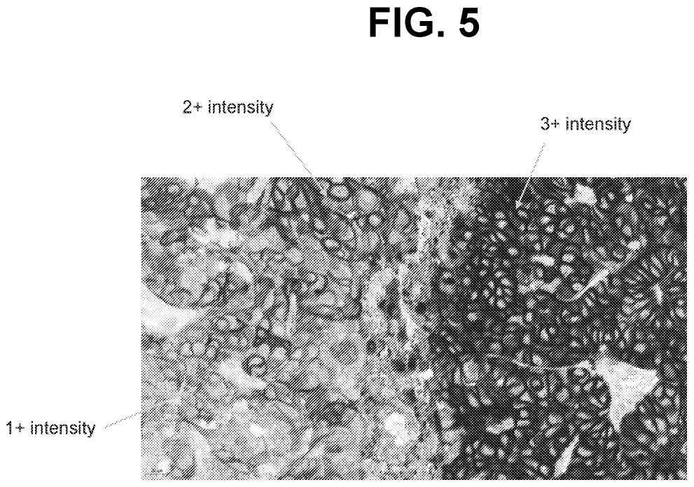

Membrane FRα expression can be measured by immunohistochemistry (IHC) and given a “staining intensity score” and/or a “staining uniformity score” by comparison to calibrated controls exhibiting defined scores (e.g., an intensity score of 3+ is given to the test sample if the intensity is comparable to the level 3+ calibrated control or an intensity of 2+ is given to the test sample if the intensity is comparable to the level 2+ calibrated control). A score of 0 refers to no staining. A score of 1+ refers to light (brown) staining. A score of 2+ refers to medium (brown) staining, and a score of 3+ refers to dark (brown) staining. Examples of 1+, 2+, and 3+ staining are shown in FIG. 5. Staining uniformity can be expressed as percentage (%) of cells staining at a certain intensity (e.g., 50% of cells staining at intensity of 1+, 2+, or 3+). With regard to membrane FRα expression by IHC, “PS” refers to percentage stained. Thus, for example, “≥75% of cells with PS2+ staining intensity” indicates that at least 75% of cells in sample have a staining intensity of at least 2+(i.e., 2+ or 3+). Methods of measuring membrane FRα expression by IHC are disclosed, for example, in WO 2012/135675 and WO 2015/031815, each of which in herein incorporated by reference in its entirety.

Methods of measuring soluble FRα are known in the art and are disclosed, for example, in WO 2019/050935 and WO 2012/061759, each of which is herein incorporated by reference in its entirety. Commercial kits that can be used to measure soluble FRα are also available (e.g., Human FOLR1 Quantikine© ELISA Kit (R&D systems)).

A “reference sample” can be used to correlate and compare the results obtained with a test sample. Reference samples can be cells (e.g., cell lines, cell pellets), bodily fluids, or tissue. The FRα levels in the “reference sample” can be an absolute or relative amount, a range of amount, a minimum and/or maximum amount, a mean amount, and/or a median amount of FRα. A “reference sample” can also serve as a baseline of FRα expression to which the test sample is compared. The “reference sample” can include a prior sample or baseline sample from the same patient, a normal reference, or a reference from a relevant patient population. Generally, FRα levels are expressed as values in a standard curve. A standard curve is a quantitative method of plotting assay data to determine the concentration of FRα in a sample. In some aspects, a reference sample is an antigen standard comprising purified FRα or FRα-Fc. The methods of detection disclosed herein may involve a comparison between expression levels of FRα in a test sample and a “reference value” or “reference level.” In some aspects, the reference value is the expression level of the FRα in a reference sample. A reference value can be a predetermined value and can also be determined from reference samples (e.g., control biological samples) tested in parallel with the test samples. A reference value can be a single cut-off value, such as a median or mean or a range of values, such as a confidence interval. Reference values can be established for various subgroups of individuals, such as individuals predisposed to cancer, individuals having early or late stage cancer, male and/or female individuals, or individuals undergoing cancer therapy.

A “biological sample” is of biological origin, in some aspects, such as from eukaryotic organisms. In some aspects, the sample is a human sample, but animal samples may also be used. Non-limiting sources of a sample for use include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example. The description provided herein is useful for cancer samples, including e.g., cancer samples that comprise bodily fluids such as ascites, where the amount of available material is small.

As used herein, the term “capture reagent” refers to a reagent capable of binding and capturing a target molecule in a sample such that under suitable condition, the capture reagent-target molecule complex can be separated from the rest of the sample. The term “immunocapture reagent” refers to an immunological reagent that is capable of binding and capturing a target molecule in a sample such that under suitable conditions, the capture reagent-target molecule complex can be separated from the rest of the sample. In some aspects, the immunocapture reagent is an antibody or antigen-binding fragment. In some aspects, the capture reagent or immunocapture reagent is immobilized. In some aspects, the capture reagent or immunocapture reagent is immobilized on a solid support.

As used herein, the term “detectable antibody” refers to an antibody that is capable of being detected either directly through a label amplified by a detection means, or indirectly through, e.g., another antibody that is labeled. For direct labeling, the antibody is typically conjugated to a moiety that is detectable by some means. In some aspects, the detectable antibody is a biotinylated antibody.

The word “label” when used herein refers to a detectable compound or composition which is conjugated directly or indirectly to the antibody so as to generate a “labeled” antibody. The label can be detectable by itself (e.g., radioisotope labels or fluorescent labels) or, in the case of an enzymatic label, can catalyze chemical alteration of a substrate compound or composition which is detectable.

By “correlate” or “correlating” is meant comparing, in any way, the performance and/or results of a first analysis with the performance and/or results of a second analysis. For example, one may use the results of a first analysis in carrying out the second analysis and/or one may use the results of a first analysis to determine whether a second analysis should be performed and/or one may compare the results of a first analysis with the results of a second analysis

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. As used herein, such antibodies include, for example, monospecific and bispecific (e.g., biparatopic) antibodies. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins (e.g., in an immunoconjugate), radioisotopes, etc.

The term “antibody fragment” or “antibody fragment thereof” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies (scFv). Antibody fragments can be naked or conjugated to other molecules such as toxins (e.g., in an immunoconjugate), radioisotopes, etc.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

A “constant” region of an antibody is not involved directly in binding an antibody to an antigen, but exhibits various effector functions, such as participation of the antibody in antibody-dependent cellular toxicity

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. 5th Ed., 1991, National Institutes of Health, Bethesda, Md.), (“Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better,” the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition enzyme-linked immunosorbent assays (ELISAs). An antibody may be said to competitively inhibit binding of the reference antibody to a given epitope by at least 90%, at least 80%, at least 70%, at least 60%, or at least 50%.

The phrase “substantially similar,” or “substantially the same,” as used herein, denotes a sufficiently high degree of similarity between two numeric values (generally one associated with an antibody of the disclosure and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

The terms “polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, aspects wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and optionally expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

“Bispecific antibodies” refer to antibodies that bind to two different epitopes. The epitopes can be on the same target antigen or can be on different target antigens.

“Biparatopic antibodies” are bispecific antibodies that bind to two different non-overlapping epitopes on the same target antigen (e.g., FRα).

In some aspects, the FRα antibodies or antigen binding fragments thereof disclosed herein are multivalent molecules. The term “valent” as used within the current application denotes the presence of a specified number of binding sites in an antibody molecule. A natural antibody for example or a full length antibody has two binding sites and is “bivalent.” The term “tetravalent,” denotes the presence of four binding sites in an antigen binding protein. The term “trivalent” denotes the presence of three binding sites in an antibody molecule. The term “bispecific, tetravalent,” as used herein denotes an antigen binding protein that has four antigen-binding sites of which at least one binds to a first antigen and at least one binds to a second antigen or another epitope of the antigen.

The term “immunoconjugate” or “conjugate” as used herein refers to a compound or a derivative thereof that is linked to a cell binding agent and is defined by a generic formula: C-L-A, wherein C=cytotoxin, L=linker, and A=antibody or antigen-binding fragment there of (e.g., an anti-FRα antibody or antibody fragment). Immunoconjugates can also be defined by the generic formula in reverse order: A-L-C.

A “linker” is any chemical moiety that is capable of linking a compound, usually a drug, such as maytansinoid, to a cell-binding agent such as an anti-FRα antibody or antigen-binding fragment thereof in a stable, covalent manner. Linkers can be susceptible to or be substantially resistant to cleavage (e.g., acid-induced cleavage, light-induced cleavage, peptidase-induced cleavage, esterase-induced cleavage, or disulfide bond cleavage) at conditions under which the compound or the antibody remains active. Suitable linkers are well known in the art and include, for example, disulfide groups and thioether groups.

As used herein, the term “cytotoxic agent” refers to a substance that inhibits or prevents one or more cellular functions and/or causes cell death. In some aspects, the cytotoxic agent is a maytansinoid, e.g., DM4 or DM21. Immunoconjugates comprising DM4 and DM21 are disclosed in WO 2011/106528 and WO 2018/160539 A1, each of which is herein incorporated by reference in its entirety.

An immunoconjugate can comprise the site-specific DM21 linkage of “DM21C” represented by the following structural formula:

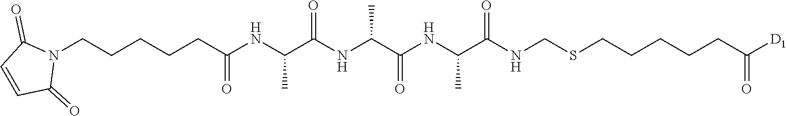

wherein D₁ is:

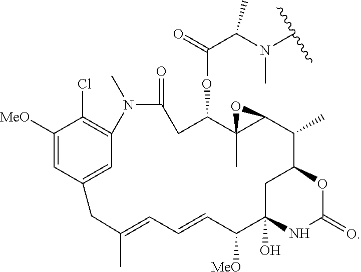 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

An immunoconjugate can also comprise the lysine-linked DM21 “L-DM21,” “DM21-L.” “DM21L,” or “DM21L-G” which are represented by the following structural formula:

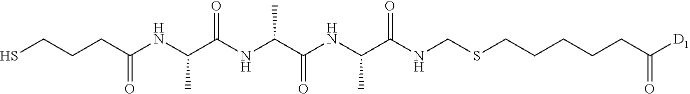

wherein D₁ is shown above, coupled to an antibody by a linker, e.g., a γ-maleimidobutyric acid N-succinimidyl ester (GMBS) or a N-(γ-maleimidobutryloxy)sulfosuccinimide ester (sulfo-GMBS or sGMBS) linker. The GMBS and sulfo-GMBS (or sGMBS) linkers are known in the art and can be presented by the following structural formula:

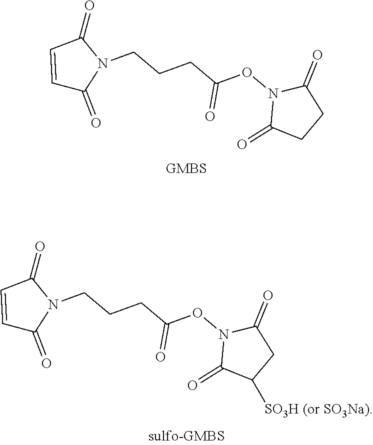

“Optional” or “optionally” means that the subsequently described circumstance may or may not occur, so that the application includes instances where the circumstance occurs and instances where it does not. For example, the phrase “optionally substituted” means that a nonhydrogen substituent may or may not be present on a given atom, and, thus, the application includes structures wherein a non-hydrogen substituent is present and structures wherein a nonhydrogen substituent is not present.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include fallopian tube cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. The cancer can be a cancer that expresses FRα (“FRα-expressing cancer”).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

An “advanced” cancer is one which has spread outside the site or organ of origin, either by local invasion or metastasis. The term “advanced” cancer includes both locally advanced and metastatic disease.

“Metastatic” cancer refers to cancer that has spread from one part of the body) to another part of the body.

A “refractory” cancer is one that progresses even though an anti-tumor treatment, such as a chemotherapy, is administered to the cancer patient.

A “recurrent” cancer is one that has regrown, either at the initial site or at a distant site, after a response to initial therapy.

A “relapsed” patient is one who has signs or symptoms of cancer after remission. Optionally, the patient has relapsed after adjuvant or neoadjuvant therapy.

The term “maintenance therapy” refers to therapy that is given to help keep cancer from coming back after it has disappeared following the initial therapy.

The terms “subject” and “patient” refer to any animal (e.g., a mammal), including, but not limited to humans, non-human primates, rodents, and the like, which is to be the recipient of a particular treatment.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an antibody, immunoconjugate, or other drug as disclosed herein is an amount sufficient to carry out a specifically stated purpose.

The term “therapeutically effective amount” refers to an amount of an antibody, immunoconjugate, or other drug effective to “treat” a disease or disorder in a subject or mammal. In the case of cancer, the therapeutically effective amount of the drug can reduce the number of cancer cells; reduce the tumor size or burden; inhibit (i.e., slow to some extent and in some aspects, stop) cancer cell infiltration into peripheral organs; inhibit (i.e., slow to some extent and in some aspects, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. See the definition herein of “treating”. To the extent the drug can prevent growth and/or kill existing cancer cells, it can be cytostatic and/or cytotoxic.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer according to the methods of the present disclosure if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

The terms “administer,” “administering,” “administration,” and the like, as used herein, refer to methods that may be used to enable delivery of the immunoconjugate to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. In one aspect, immunoconjugate is administered intravenously.

The term “instructing” means providing directions for applicable therapy, medication, treatment, treatment regimens, and the like, by any means, for example, in writing, such as in the form of package inserts or other written promotional material.

The terms “pre-treat” and “pre-treatment” refer to therapeutic measures that occur prior to the administration of a therapeutic antibody, antigen-binding fragment thereof, or immunoconjugate. For example, as described in more detail herein, a steroid (e.g., corticosteroid) can be administered as a prophylactic within about a week, about five days, about three days, about two days, or about one day or 24 hours prior to the administration of an anti-FRα active agent (e.g., an anti-FRα immunoconjugate). The steroid can also be administered prior to the anti-FRα active agent (e.g., anti-FRα immunoconjugate) on the same day as the anti-FRα active agent (e.g., anti-FRα immunoconjugate).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

The recitation of a listing of chemical groups in any definition of a variable herein includes definitions of that variable as any single group or combination of listed groups. The recitation of an aspect herein includes that aspect as any single aspect or in combination with any other aspects or portions thereof.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following: A, B, and C; A, B, or C; A or C; A or B; B or C; A and C; A and B; B and C; A (alone); B (alone); and C (alone).

Any compositions or methods provided herein can be combined with one or more of any of the other compositions and methods provided herein.

### II. Anti-FRα Antibodies and Antigen-Binding Fragments Thereof

As described herein, anti-FRα antibodies and antigen-binding fragments thereof can be used therapeutically (e.g., to treat cancer) and/or to detect FRα (e.g., soluble FRα and/or membrane FRα). Exemplary anti-FRα antibodies and antigen binding fragments thereof are known in the art and have been disclosed, for example, in WO 2011/106528, WO 2014/036495, WO 2015/031815, and U.S. Published Application No. 2020/0362029, each of which is herein incorporated by reference in its entirety. Additional anti-FRα antibodies and antigen binding fragments thereof are known in the art and have been disclosed, for example, in WO 2012/061759, U.S. Published Application No. 2019/0233512, U.S. Published Application No. 2020/0147229, U.S. Published Application No. 2020/0297860 U.S. Published Application No. 2020/0353076, U.S. Pat. Nos. 10,822,410 and 10,101,343, each of which is herein incorporated by reference in its entirety.

In addition, the anti-FRα antibody huMov19 (M9346A) is encoded by the plasmids deposited with the American Type Culture Collection (ATCC), located at 10801 University Boulevard, Manassas, VA 20110 on Apr. 7, 2010 under the terms of the Budapest Treaty and having ATCC deposit nos. PTA-10772 and PTA-10774. In addition, a biparatopic anti-FRα antibody is encoded by the plasmids deposited with the American Type Culture Collection (ATCC) and having ATCC deposit nos. PTA-10774 (deposited in Apr. 7, 2010), PTA-125915 (“Mov19-Fc-hole”; deposited to the ATCC on Apr. 29, 2019 and received by the ATCC on Apr. 30, 2019), and PTA-125916 (“FR57scFv2-Fc-knob”; deposited to the ATCC on Apr. 29, 2019 and received by the ATCC on Apr. 30, 2019).

By way of example, an FRα-antibody or antigen-binding fragment thereof can comprise the six CDR sequences (i.e., the VH-CDR1, VH-CDR2, VH-CDR3, VL-CDR1, VL-CDR2, and VL-CDR3) of the huMov19 antibody and/or the FR57 antibody or a variant thereof, the mu1-9 antibody, the mu1-13 antibody, or the 2.1 antibody. An FRα-antibody or antigen-binding fragment thereof can comprise the six CDR sequences (i.e., the VH-CDR1, VH-CDR2, VH-CDR3, VL-CDR1, VL-CDR2, and VL-CDR3) of the MORAb-003 antibody (also known as farletuzumab) and/or the 1848-H01 antibody. The CDR sequences can be the Kabat-defined CDRs, the Chothia-defined CDRs, the AbM-defined CDRs, or a mixture thereof. The CDR sequences can be the IMGT-defined CDRs. CDR sequences of huMov19, FR57 and variants thereof, 1-9, 1-13, and 2.1 are provided in Tables 1 and 2 below. CDR sequences of MORAb-003 and 1848-H01 are also provided in Tables 1 and 2 below.

TABLE 1
Heavy chain CDR sequences

| Antibody | VH-CDR1 | VH-CDR2 | VH-CDR3 |
|---|---|---|---|
| FR57 | SFGMH (SEQ ID | Kabat Defined: | Kabat or AbM Defined: |
| | NO: 2) | YISSGSSTISYADSVKG | EAYGSSMEY (SEQ ID |
| | AbM Defined: | (SEQ ID NO: 3) | NO: 4) |
| | GFTFSSFGMH | AbM Defined: | |
| | (SEQ ID NO: 5) | YISSGSSTIS (SEQ ID | |
| | | NO: 6) | |
| huMov19 | Kabat Defined: | Kabat Defined: | Kabat or AbM Defined: |
| | GYFMN (SEQ ID | RIHPYDGDTFYNQKFQG | YDGSRAMDY (SEQ ID |
| | NO: 10) | (SEQ ID NO: 11) | NO: 12) |
| | AbM Defined: | AbM Defined: | |
| | GYTFTGYFMN | RIHPYDGDTF (SEQ ID | |
| | (SEQ ID NO: 13) | NO: 14) | |
| muFR1-9 | SFGMH (SEQ ID | YISSGSSTFYYADTVKG | ELTGTFAY (SEQ ID |
| | NO: 18) | (SEQ ID NO: 19) | NO: 20) |
| muFR1-13 | RYSVH (SEQ ID | MIWSGGNTDYNSVFKS | FDGKVSWFAY (SEQ |
| | NO: 24) | (SEQ ID NO: 25) | ID NO: 26) |
| muFRIHC2- | NSYIH (SEQ ID | WIYPESLNTQYNEKFKA | RGIYYYSPYALDH |
| 1 (″2.1″) | NO: 30) | (SEQ ID NO: 31) | (SEQ ID NO: 32) |
| MORAb- | Kabat Defined: | Kabat Defined: | Kabat Defined: |
| 003 | GYGLS (SEQ ID | MISSGGSYTYYADSVKG | HGDDPAWFAY (SEQ |
| | NO: 80) | (SEQ ID NO: 81) | ID NO: 82) |
| | IMGT Defined: | IMGT Defined: | IMGT Defined: |
| | GFTFSGYG (SEQ | ISSGGSYT (SEQ ID | ARHGDDPAWFAY |
| | ID NO: 83) | NO: 84) | (SEQ ID NO: 85) |
| 1848-H01 | Kabat Defined: | Kabat Defined: | GSWSWPSGMDYYLDY |
| | TQSIH (SEQ ID | DIFPIDGITDYADSVKG | (SEQ ID NO: 93) |
| | NO: 91) | (SEQ ID NO: 92) | |
| | Chothia Defined: | Chothia Defined: FPIDGI | |
| | GFNIRTQ (SEQ ID | (SEQ ID NO: 95) | |
| | NO: 94) | | |

TABLE 2
Light chain CDR sequences

| Antibody | VL-CDR1 | VL-CDR2 | VL-CDR3 |
|---|---|---|---|
| FR57 | RASQNINNNLH (SEQ | YVSQSVS (SEQ ID | QQSNSWPHYT |
| | ID NO: 7) | NO: 8) | (SEQ ID NO: 9) |
| huMov19 | KASQSVSFAGTSLMH | RASNLEA | QQSREYPYT |
| | (SEQ ID NO: 15) | (SEQ ID NO: 16) | (SEQ ID NO: 17) |
| muFR1-9 | RASQSINNNLH (SEQ | YASQSIS (SEQ ID | QQSNSWPQVT |
| | ID NO: 21) | NO: 22) | (SEQ ID NO: 23) |
| muFR1-13 | KASQSVSNDVL (SEQ | YAYNRYS (SEQ ID | QQDHSSPFT |
| | ID NO: 27) | NO: 28) | (SEQ ID NO: 29) |
| muFRIHC2-1 | KSSKSLLNSDGFTYLD | LVSNHFS (SEQ ID | FQSNYLPLT |
| (″2.1″) | (SEQ ID NO: 33) | NO: 34) | (SEQ ID NO: 35) |
| MORAb-003 | Kabat Defined: | Kabat Defined: | Kabat or IMGT |
| | SVSSSISSNNLH (SEQ | GTSNLAS (SEQ ID | Defined: |
| | ID NO: 86) | NO: 87) | QQWSSYPYMYT |
| | IMGT Defined: | IMGT Defined: | (SEQ ID NO: 88) |
| | SSISSNN (SEQ ID | GTS (SEQ ID NO: 90) | |
| | NO: 89) | | |
| 1848-H01 | RASQDVNTAVA (SEQ | SASFLYS (SEQ ID | QQHYTTPPT |
| | ID NO: 96) | NO: 97) | (SEQ ID NO: 98) |

In some aspects, an anti-FRα˜-antibody or antigen-binding fragment thereof comprises (a) VH CDR1, VH CDR2, and VH CDR3 comprising the amino acid sequences of SEQ ID NOs:2-4, respectively; and (b) VL CDR1, VL CDR2, and VL CDR3 comprising the amino acid sequences of SEQ ID NOs:7-9, respectively. In some aspects, an anti-FRα˜-antibody or antigen-binding fragment thereof comprises (a) VH CDR1, VH CDR2, and VH CDR3 comprising the amino acid sequences of SEQ ID NOs:5, 6, and 4, respectively; and (b) VL CDR1, VL CDR2, and VL CDR3 comprising the amino acid sequences of SEQ ID NOs:7-9, respectively. In some aspects, an anti-FRα˜-antibody or antigen-binding fragment thereof comprises (a) VH CDR1, VH CDR2, and VH CDR3 comprising the amino acid sequences of SEQ ID NOs:2, 6, and 4, respectively; and (b) VL CDR1, VL CDR2, and VL CDR3 comprising the amino acid sequences of SEQ ID NOs:7-9, respectively. In some aspects, an anti-FRα-antibody or antigen-binding fragment thereof comprises (a) VH CDR1, VH CDR2, and VH CDR3 comprising the amino acid sequences of SEQ ID NOs:5, 3, and 4, respectively; and (b) VL CDR1, VL CDR2, and VL CDR3 comprising the amino acid sequences of SEQ ID NOs:7-9, respectively.

In some aspects, an anti-FRα-antibody or antigen-binding fragment thereof comprises (a) VH CDR1, VH CDR2, and VH CDR3 comprising the amino acid sequences of SEQ ID NOs:10-12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof comprises (a) VH CDR1, VH CDR2, and VH CDR3 comprising the amino acid sequences of SEQ ID NOs:13, 14, and 12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof comprises (a) VH CDR1, VH CDR2, and VH CDR3 comprising the amino acid sequences of SEQ ID NOs:10, 14, and 12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof comprises (a) VH CDR1, VH CDR2, and VH CDR3 comprising the amino acid sequences of SEQ ID NOs:13, 11, and 12, respectively; and (b) VL CDR1, VL CDR2, and VL CDR3 comprising the amino acid sequences of SEQ ID NOs:15-17, respectively.

In some aspects, an anti-FRα-antibody or antigen-binding fragment thereof comprises (a) VH CDR1, VH CDR2, and VH CDR3 comprising the amino acid sequences of SEQ ID NOs:18-20, respectively; and (b) VL CDR1, VL CDR2, and VL CDR3 comprising the amino acid sequences of SEQ ID NOs:21-23, respectively.

In some aspects, an anti-FRα-antibody or antigen-binding fragment thereof comprises (a) VH CDR1, VH CDR2, and VH CDR3 comprising the amino acid sequences of SEQ ID NOs:24-26, respectively; and (b) VL CDR1, VL CDR2, and VL CDR3 comprising the amino acid sequences of SEQ ID NOs:27-29, respectively.

In some aspects, an anti-FRα-antibody or antigen-binding fragment thereof comprises (a) VH CDR1, VH CDR2, and VH CDR3 comprising the amino acid sequences of SEQ ID NOs:30-32, respectively; and (b) VL CDR1, VL CDR2, and VL CDR3 comprising the amino acid sequences of SEQ ID NOs:33-35, respectively.

In some aspects, an anti-FRα-antibody or antigen-binding fragment thereof comprises (a) VH CDR1, VH CDR2, and VH CDR3 comprising the amino acid sequences of SEQ ID NOs:80-82, respectively; and (b) VL CDR1, VL CDR2, and VL CDR3 comprising the amino acid sequences of SEQ ID NOs:86-88, respectively.

In some aspects, an anti-FRα-antibody or antigen-binding fragment thereof comprises (a) VH CDR1, VH CDR2, and VH CDR3 comprising the amino acid sequences of SEQ ID NOs:83-85, respectively; and (b) VL CDR1, VL CDR2, and VL CDR3 comprising the amino acid sequences of SEQ ID NOs:89, 90, and 88, respectively.

In some aspects, an anti-FRα-antibody or antigen-binding fragment thereof comprises (a) VH CDR1, VH CDR2, and VH CDR3 comprising the amino acid sequences of SEQ ID NOs:91-93, respectively; and (b) VL CDR1, VL CDR2, and VL CDR3 comprising the amino acid sequences of SEQ ID NOs:96-98, respectively.

In some aspects, an anti-FRα-antibody or antigen-binding fragment thereof comprises (a) VH CDR1, VH CDR2, and VH CDR3 comprising the amino acid sequences of SEQ ID NOs:94, 95, and 93, respectively; and (b) VL CDR1, VL CDR2, and VL CDR3 comprising the amino acid sequences of SEQ ID NOs:96-98, respectively.

In some aspects, an anti-FRα-antibody or antigen-binding fragment thereof comprises the heavy and/or light chain variable sequences of the huMov19 antibody and/or the FR57 antibody or a variant thereof, the mu1-9 antibody, the mu1-13 antibody, or the 2.1 antibody. In some aspects, an anti-FRα-antibody or antigen-binding fragment thereof comprises the heavy and/or light chain variable sequences of the MORAb-003 antibody or the 1848-H01 antibody. The heavy chain variable sequences and light chain variable sequences of huMov19, FR57 and variants thereof, 1-9, 1-13, and 2.1 are provided in Tables 3 and 4 below. The heavy chain variable sequences and light chain variable sequences of MORAb-003 and 1848-H01 are also provided in Tables 3 and 4 below

TABLE 3
Heavy Chain Variable (VH) Sequence

| Antibody | VH Sequence (SEQ ID NO) |
|---|---|
| FR57 | EVQLVESGGGLVQPGGSRRLSCAASGFTFSSFGMHWVRQAPGKGLEWV |
| | AYISSGSSTISYADSVKGRFTISRDNSKKTLLLQMTSLRAEDTAMYYCAR |
| | EAYGSSMEYWGQGTLVTVSS (SEQ ID NO: 36) |
| FR57 | EVQLVQSGGGLVQPGGSRRLSCAASGFTFSSFGMHWVRQAPGKCLEWV |
| E6Q; | AYISSGSSTISYADSVKGRFTISRDNSKKTLLLQMTSLRAEDTAMYYCAR |
| G44C | EAYGSSMEYWGQGTLVTVSS (SEQ ID NO: 37) |
| huMov19 | QVQLVQSGAEVVKPGASVKISCKASGYTFTGYFMNWVKQSPGQSLEWI |
| | GRIHPYDGDTFYNQKFQGKATLTVDKSSNTAHMELLSLTSEDFAVYYCT |
| | RYDGSRAMDYWGQGTTVTVSS (SEQ ID NO: 38) |
| muFR1-9 | QVQLVESGGGLVQPGGSRKLSCAASGFTFSSFGMHWVRQAPEKGLEWV |
| | AYISSGSSTFYYADTVKGRFTISRDNPKNTLFLQMTSLRSEDTAMYYCAK |
| | ELTGTFAYWGQGTLVTVSA (SEQ ID NO: 39) |
| muFR1- | QVQLKESGPDLVAPSQSLSITCTVSGFSLSRYSVHWIRQPPGKGLEWLGM |
| 13 | IWSGGNTDYNSVFKSRLNITKDNSKSQVFLKMNSLQTDDTAIYYCATFD |
| | GKVSWFAYWGQGTLVTVSA (SEQ ID NO: 40) |
| muFRIHC2-1 | QVQLQQSGPELVKPGASVRISCKASGYTFTNSYIHWVKKRPGQGLEWIG |
| (″2.1″) | WIYPESLNTQYNEKFKAKATLTADKSSSTSYMQLSSLTSEDSAVYFCARR |
| | GIYYYSPYALDHWGQGASVTVSS (SEQ ID NO: 41) |
| MORAb- | EVQLVESGGGVVQPGRSLRLSCSASGFTFSGYGLSWVRQAPGKGLEWV |
| 003 | AMISSGGSYTYYADSVKGRFAISRDNAKNTLFLQMDSLRPEDTGVYFCA |
| | RHGDDPAWFAYWGQGTPVTVSS (SEQ ID NO: 99) |
| 1848-H01 | EVQLVESGGGLVQPGGSLRLSCAASGFNIRTQSIHWVRQAPGKGLEWIG |
| | DIFPIDGITDYADSVKGRFTISADTSKNTAYLQMNSLRAEDTAVYYCARG |
| | SWSWPSGMDYYLDYWGQGTLVTVSS (SEQ ID NO: 100) |

TABLE 4
Light Chain Variable (VL) Sequence

| Antibody | VL Sequence (SEQ ID NO) |
|---|---|
| FR57 | EIVLTQSPATLSVTPGDRVSLSCRASQNINNNLHWYQQKPGQSPRLLIK |
| | YVSQSVSGIPDRFSGSGSGTDFTLSISSVEPEDFGMYFCQQSNSWPHYT |
| | FGQGTKLEIK (SEQ ID NO: 42) |
| FR57 F83E; | EIVLTQSPATLSVTPGDRVSLSCRASQNINNNLHWYQQKPGQSPRLLIK |
| Q101C | YVSQSVSGIPDRFSGSGSGTDFTLSISSVEPEDEGMYFCQQSNSWPHYT |
| | FGCGTKLEIK (SEQ ID NO: 43) |
| huMov19 | DIVLTQSPLSLAVSLGQPAIISCKASQSVSFAGTSLMHWYHQKPGQQPR |
| | LLIYRASNLEAGVPDRFSGSGSKTDFTLTISPVEAEDAATYYCQQSREY |
| | PYTFGGGTKLEIK (SEQ ID NO: 44) |
| muFR1-9 | DIVLTQSPATLSVTPGDSVSLSCRASQSINNNLHWYQQKSHESPRLLIK |
| | YASQSISGIPSRFSGSGSGTDFTLSINSVETEDFGMYFCQQSNSWPQVTF |
| | GAGTKLELKR (SEQ ID NO: 45) |
| muFR1-13 | SIVMTQTPKFLLVSTGDRFTITCKASQSVSNDVLWYQQKPGQSPKLLIY |
| | YAYNRYSGVPDRFTGSGYGTDFTFTITTVQSEDLAVYFCQQDHSSPFT |
| | FGSGTKLEIKR (SEQ ID NO: 46) |
| muFRIHC2-1 | SDVVLTQTPLSLPVNIGDQASISCKSSKSLLNSDGFTYLDWYLQKPGQS |
| (″2.1″) | PQLLIYLVSNHFSGVPDRFSGSGSGTDFTLKISRVEAEDLGVYYCFQSN |
| | YLPLTFGGGTKLEIKR (SEQ ID NO: 47) |
| MORAb- | DIQLTQSPSSLSASVGDRVTITCSVSSSISSNNLHWYQQKPGKAPKPWI |
| 003 | YGTSNLASGVPSRFSGSGSGTDYTFTISSLQPEDIATYYCQQWSSYPYM |
| | YTFGQGTKVEIK (SEQ ID NO: 101) |
| 1848-H01 | DIQMTQSPSSLSASVGDRVTITCRASQDVNTAVAWYQQKPGKAPKLLI |
| | YSASFLYSGVPSRFSGSRSGTDFTLTISSLQPEDFATYYCQQHYTTPPTF |
| | GQGTKVEIK (SEQ ID NO: 102) |

In some aspects, an anti-FRα˜-antibody or antigen-binding fragment thereof comprises the heavy and/or light chain sequences of the huMov19 antibody and/or the FR57 antibody or a variant thereof, the mu1-9 antibody, the mu1-13 antibody, or the 2.1 antibody. In some aspects, an anti-FRα˜-antibody or antigen-binding fragment thereof comprises the heavy and/or light chain sequences of the MORAb-003 antibody. The heavy chain sequences and light chain variable sequences of huMov19, FR57 and variants thereof, 1-9, 1-13, and 2.1 are provided in Tables 5 and 6 below. The heavy chain sequences and light chain variable sequences of MORAb-003 are also provided in Tables 5 and 6 below.

TABLE 5
Full-length heavy chain amino acid sequences

| Antibody | Full-Length Heavy Chain Amino Acid Sequence (SEQ ID NO) |
|---|---|
| FR57 | EVQLVESGGGLVQPGGSRRLSCAASGFTFSSFGMHWVRQAPGKGLE |
| | WVAYISSGSSTISYADSVKGRFTISRDNSKKTLLLQMTSLRAEDTAM |
| | YYCAREAYGSSMEYWGQGTLVTVSSASTKGPSVFPLAPSSKSTSGG |
| | TAALGCLVKDYFPEPVTVSWNSGALTSGVHTFPAVLQSSGLYSLSSV |
| | VTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQ |
| | QGNVFSCSVMHEALHNHYTQKSLSLSPG (SEQ ID NO: 48) |
| FR57 E6Q; | EVQLVQSGGGLVQPGGSRRLSCAASGFTFSSFGMHWVRQAPGKCL |
| G44C | EWVAYISSGSSTISYADSVKGRFTISRDNSKKTLLLQMTSLRAEDTA |
| | MYYCAREAYGSSMEYWGQGTLVTVSSASTKGPSVFPLAPSSKSTSG |
| | GTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNW |
| | YVDGVEVHNAKTKPREEQYNSTYRVVSVLTVLHQDWLNGKEYKC |
| | KVSNKALPAPIEKTISKAKGQPREPQVYTLPPSRDELTKNQVSLTCL |
| | VKGFYPSDIAVEWESNGQPENNYKTTPPVLDSDGSFFLYSKLTVDK |
| | SRWQQGNVFSCSVMHEALHNHYTQKSLSLSPG (SEQ ID NO: 49) |
| huMov19 | QVQLVQSGAEVVKPGASVKISCKASGYTFTGYFMNWVKQSPGQSL |
| | EWIGRIHPYDGDTFYNQKFQGKATLTVDKSSNTAHMELLSLTSEDF |
| | AVYYCTRYDGSRAMDYWGQGTTVTVSSASTKGPSVFPLAPSSKST |
| | SGGTAALGCLVKDYFPEPVTVSWNSGALTSGVHTFPAVLQSSGLYS |
| | LSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPAPIEKTISKAKGQPREPQVYTLPPSRDELTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPG (SEQ ID |
| | NO: 50) |
| muFR1-9 | QVQLVESGGGLVQPGGSRKLSCAASGFTFSSFGMHWVRQAPEKGL |
| | EWVAYISSGSSTFYYADTVKGRFTISRDNPKNTLFLQMTSLRSEDTA |
| | MYYCAKELTGTFAYWGQGTLVTVSAAKTTPPSVYPLAPGSAAQTN |
| | SMVTLGCLVKGYFPEPVTVTWNSGSLSSGVHTFPAVLESDLYTLSS |
| | SVTVPSSMRPSETVTCNVAHPASSTKVDKKIVPRDCGCKPCICTVPE |
| | VSSVFIFPPKPKDVLTITLTPKVTCVVVDISKDDPEVQFSWFVDDVE |
| | VHTAQTQPREEQFNSTFRSVSELPIMHQDWLNGKEFKCRVNSAAFP |
| | APIEKTISKTKGRPKAPQVYTIPPPKEQMAKDKVSLTCMITDFFPEDI |
| | TVEWQWNGQPAENYKNTQPIMNINGSYFVYSKLNVQKSNWEAGN |
| | TFTCSVLHEGLHNHHTEKSLSHSPGK (SEQ ID NO: 51) |
| muFR1-13 | QVQLKESGPDLVAPSQSLSITCTVSGFSLSRYSVHWIRQPPGKGLEW |
| | LGMIWSGGNTDYNSVFKSRLNITKDNSKSQVFLKMNSLQTDDTAIY |
| | YCATFDGKVSWFAYWGQGTLVTVSAAKTTPPSVYPLAPGCGDTTG |
| | SSVTLGCLVKGYFPESVTVTWNSGSLSSSVHTFPALLQSGLYTMSSS |
| | VTVPSSTWPSQTVTCSVAHPASSTTVDKKLEPSGPISTINPCPPCKEC |
| | HKCPAPNLEGGPSVFIFPPNIKDVLMISLTPKVTCVVVDVSEDDPDV |
| | QISWFVNNVEVHTAQTQTHREDYNSTIRVVSTLPIQHQDWMSGKEF |
| | KCKVNNKDLPSPIERTISKIKGLVRAPQVYILPPPAEQLSRKDVSLTC |
| | LVVGFNPGDISVEWTSNGHTEENYKDTAPVLDSDGSYFIYSKLNMK |
| | TSKWEKTDSFSCNVRHEGLKNYYLKKTISRSPGK (SEQ ID NO: 52) |
| muFRIHC2-1 | QVQLQQSGPELVKPGASVRISCKASGYTFTNSYIHWVKKRPGQGLE |
| (″2.1″) | WIGWIYPESLNTQYNEKFKAKATLTADKSSSTSYMQLSSLTSEDSA |
| | VYFCARRGIYYYSPYALDHWGQGASVTVSSAKTTPPSVYPLAPGSA |
| | AQTNSMVTLGCLVKGYFPEPVTVTWNSGSLSSGVHTFPAVLESDLY |
| | TLSSSVTVPSSMRPSETVTCNVAHPASSTKVDKKIVPRDCGCKPCIC |
| | TVPEVSSVFIFPPKPKDVLTITLTPKVTCVVVDISKDDPEVQFSWFVD |
| | DVEVHTAQTQPREEQFNSTFRSVSELPIMHQDWLNGKEFKCRVNSA |
| | AFPAPIEKTISKTKGRPKAPQVYTIPPPKEQMAKDKVSLTCMITDFFP |
| | EDITVEWQWNGQPAENYKNTQPIMNTNGSYFVYSKLNVQKSNWE |
| | AGNTFTCSVLHEGLHNHHTEKSLSHSPGK (SEQ ID NO: 53) |
| MORAb-003 | EVQLVESGGGVVQPGRSLRLSCSASGFTFSGYGLSWVRQAPGKGLE |
| | WVAMISSGGSYTYYADSVKGRFAISRDNAKNTLFLQMDSLRPEDT |
| | GVYFCARHGDDPAWFAYWGQGTPVTVSSASTKGPSVFPLAPSSKS |
| | TSGGTAALGCLVKDYFPEPVTVSWNSGALTSGVHTFPAVLQSSGLY |
| | SISSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSRDELTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGK (SEQ |

NO: 103)

TABLE 6
Full-length light chain amino acid sequences

| Antibody | Full-Length Light Chain Amino Acid Sequence (SEQ ID NO) |
|---|---|
| FR57 | EIVLTQSPATLSVTPGDRVSLSCRASQNINNNLHWYQQKPGQSPRLLI |
| | KYVSQSVSGIPDRFSGSGSGTDFTLSISSVEPEDFGMYFCQQSNSWPH |
| | YTFGQGTKLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC (SEQ ID NO: 54) |
| FR57 F83E; | EIVLTQSPATLSVTPGDRVSLSCRASQNINNNLHWYQQKPGQSPRLL |
| Q101C | IKYVSQSVSGIPDRFSGSGSGTDFTLSISSVEPEDEGMYFCQQSNSWP |
| | HYTFGCGTKLEIKRTVAAPSVFIFPPSDEQLKSGTASVVCLLNNFYP |
| | REAKVQWKVDNALQSGNSQESVTEQDSKDSTYSLSSTLTLSKADY |
| | EKHKVYACEVTHQGLSSPVTKSFNRGEC (SEQ ID NO: 55) |
| huMov19 | DIVLTQSPLSLAVSLGQPAIISCKASQSVSFAGTSLMHWYHQKPGQQ |
| | PRLLIYRASNLEAGVPDRFSGSGSKTDFTLTISPVEAEDAATYYCQQ |
| | SREYPYTFGGGTKLEIKRTVAAPSVFIFPPSDEQLKSGTASVVCLLN |
| | NFYPREAKVQWKVDNALQSGNSQESVTEQDSKDSTYSLSSTLTLSK |
| | ADYEKHKVYACEVTHQGLSSPVTKSFNRGEC (SEQ ID NO: 56) |
| muFR1-9 | DIVLTQSPATLSVTPGDSVSLSCRASQSINNNLHWYQQKSHESPRLLI |
| | KYASQSISGIPSRFSGSGSGTDFTLSINSVETEDFGMYFCQQSNSWPQ |
| | VTFGAGTKLELKRADAAPTVSIFPPSSEQLTSGGASVVCFLNNFYPK |
| | DINVKWKIDGSERQNGVLNSWTDQDSKDSTYSMSSTLTLTKDEYE |
| | RHNSYTCEATHKTSTSPIVKSFNRNEC (SEQ ID NO: 57) |
| muFR1-13 | SIVMTQTPKFLLVSTGDRFTITCKASQSVSNDVLWYQQKPGQSPKL |
| | LIYYAYNRYSGVPDRFTGSGYGTDFTFTITTVQSEDLAVYFCQQDH |
| | SSPFTFGSGTKLEIKRADAAPTVSIFPPSSEQLTSGGASVVCFLNNFY |
| | PKDINVKWKIDGSERQNGVLNSWTDQDSKDSTYSMSSTLTLTKDE |
| | YERHNSYTCEATHKTSTSPIVKSFNRNEC (SEQ ID NO: 58) |
| muFRIHC2-1 | SDVVLTQTPLSLPVNIGDQASISCKSSKSLLNSDGFTYLDWYLQKPG |
| (″2.1″) | QSPQLLIYLVSNHFSGVPDRFSGSGSGTDFTLKISRVEAEDLGVYYC |
| | FQSNYLPLTFGGGTKLEIKRADAAPTVSIFPPSSEQLTSGGASVVCFL |
| | NNFYPKDINVKWKIDGSERQNGVLNSWTDQDSKDSTYSMSSTLTL |
| | TKDEYERHNSYTCEATHKTSTSPIVKSFNRNEC (SEQ ID NO: 59) |
| MORAb-003 | DIQLTQSPSSLSASVGDRVTITCSVSSSISSNNLHWYQQKPGKAPKP |
| | WIYGTSNLASGVPSRFSGSGSGTDYTFTISSLQPEDIATYYCQQWSS |
| | YPYMYTFGQGTKVEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSFNRGEC (SEQ ID NO: 104) |

In some aspects, an anti-FRα-antibody or antigen-binding fragment thereof comprises a single chain variable fragment (scFv). In some aspects, an anti-FRα-antibody or antigen-binding fragment thereof comprises a scFv comprising the variable heavy chain and variable light chain of FR57 antibody or a variant thereof.

Accordingly, in some aspects, an anti-FRα scFv comprises, from N- to C-terminus: a VL comprising the amino acid sequence of SEQ ID NO:42, a linker (e.g., a glycine-serine linker), and a VH comprising the amino acid sequence of SEQ ID NO:36. In some aspects, an anti-FRα scFv comprises, from N to C terminus: a VH comprising the amino acid sequence of SEQ ID NO:36, a linker (e.g., a glycine-serine linker), and a VL comprising the amino acid sequence of SEQ ID NO:42.

In some aspects, an anti-FRα scFv comprises, from N- to C-terminus: a VL comprising the amino acid sequence of SEQ ID NO:43, a linker (e.g., a glycine-serine linker), and a VH comprising the amino acid sequence of SEQ ID NO:37. In some aspects, an anti-FRα scFv comprises, from N to C terminus: a VH comprising the amino acid sequence of SEQ ID NO:37, a linker (e.g., a glycine-serine linker), and a VL comprising the amino acid sequence of SEQ ID NO:43.

In some aspects, an anti-FRα-antibody or antigen-binding fragment thereof comprises a scFv comprising the variable heavy chain and variable light chain of huMov19.

In some aspects, an anti-FRα scFv comprises, from N- to C-terminus: a VL comprising the amino acid sequence of SEQ ID NO:44, a linker (e.g., a glycine-serine linker), and a VH comprising the amino acid sequence of SEQ ID NO:38. In some aspects, an anti-FRα scFv comprises, from N to C terminus: a VH comprising the amino acid sequence of SEQ ID NO:38, a linker (e.g., a glycine-serine linker), and a VL comprising the amino acid sequence of SEQ ID NO:44.

Linkers that can be used to connect a VH and a VL are known in the art. For example, a linker can be a glycine-serine linker. In some aspects, the linker can be of any length and can comprise at least 1, 2, 3, 4, 5, 6, 7, 8, 9, 10, 11, 12, 13, 14, 15, 16, 17, 18, 19, 20, 25, 30, 31, 32, 33, 34, 35, 36, 37, 38, 39, 40, 50, or 60 or more amino acids. In some aspects, a linker useful for the present disclosure has at least one amino acid and less than 100 amino acids, less than 90 amino acids, less than 80 amino acids, less than 70 amino acids, less than 60 amino acids, less than 50 amino acids, less than 40 amino acids, less than 30 amino acids, less than 20 amino acids, less than 19 amino acids, less than 18 amino acids, less than 17 amino acids, less than 16 amino acids, less than 15 amino acids, less than 14 amino acids, less than 13 amino acids, or less than 12 amino acids. In some aspects, the linker sequence comprises glycine amino acid residues. In some aspects, the linker sequence comprises a combination of glycine and serine amino acid residues.

In some aspects, anti-FRα scFv comprises a linker fused in frame between the VH and the VL. In some aspects, such glycine/serine linkers comprises any combination of the amino acid residues, including, but not limited to, the peptide GGGS (SEQ ID NO:64) or GGGGS (SEQ ID NO:65) or repeats of the same, including 1, 2, 3, 4, 5, 6, 7, 8, 9, 10 or more repeats of these given peptides. The glycine/serine linkers disclosed herein comprises an amino acid sequence of (GS)ₙ, (GGS)ₙ, (GGGS)ₙ, (GGGGS)ₙ, or (GGGGS)ₙ, wherein n is an integer of 1, 2, 3, 4, 5, 6, 7, 8, 9, or 10. In some aspects, the linker sequence is GGGGSGGGGSGGGGS (SEQ ID NO:66) (also noted as (Gly₄Ser)₃). In some aspects, the linker sequence is GGGGSGGGGSGGGGSGGGGS (SEQ ID NO:67) (also noted as (Gly₄Ser)₄).

An exemplary sequence of an scFv comprising the variable heavy chain and variable light chain of FR57 antibody or a variant thereof is provided in Table 7 below.

TABLE 7
ScFv sequence

| Name | scFv Sequence (SEQ ID NO) |
|---|---|
| FR57scFv | EIVLTQSPATLSVTPGDRVSLSCRASQNINNNLHWYQQKPGQSPRLLIK |
| scFv in VL | YVSQSVSGIPDRFSGSGSGTDFTLSISSVEPEDEGMYFCQQSNSWPHYT |
| (F83E; | FGCGTKLEIKGGGGSGGGGSGGGGSGGGGSEVQLVQSGGGLVQPGGS |
| Q101C)-(G₄S)₄- | RRLSCAASGFTFSSFGMHWVRQAPGKCLEWVAYISSGSSTISYADSVK |
| VH (E6Q; G44C) | GRFTISRDNSKKTLLLQMTSLRAEDTAMYYCAREAYGSSMEYWGQGT |
| orientation | LVTVSS (SEQ ID NO: 60) |

In some aspects, an anti-FRα-antibody or antigen-binding fragment thereof is a biparatopic antibody or antigen-binding fragment thereof. Many different types of bispecific constructs are known in the art and can be used in suck biparatopic anti-FRα antibodies or antigen binding fragments.

In some aspects, a biparatopic anti-FRα-antibody or antigen-binding fragment thereof is a construct with asymmetric-Fc molecules, including in “knob-in-hole” structures. See Kontermann, MAbs., 4(2):182-97 (2012). Knobs-into-holes (KIHs) technology involves engineering C_{H}3 domains to create either a “knob” or a “hole” in each heavy chain to promote heterodimerization. KIH technology is described, for instance, in Ridgway et al., Protein Engineering 9(7):617-721 (1996); U.S. Pat. Nos. 5,731,168; 5,807,706; 5,821,333, each of which is herein incorporated by reference in its entirety. The “CrossMab” technique further involves the exchange of heavy and light chain domains within the Fab of one half of the bispecific antibody, making the two arms so different that light-heavy chain mispairing cannot occur (Schaefer et al., 2011, Proc Natl. Acad Sci USA 108:11187-92). The knobs-into-holes approach introduces amino acids with bulky side chains into the CH3 domain of one heavy chain that fit into appropriately designed cavities in the CH3 domain of the other heavy chain. The combination of approaches prevents mismatch of both heavy chain to heavy chain and heavy chain to light chain interactions, resulting in primarily a single product.

In some aspects, a biparatopic anti-FRα antibody or antigen binding fragment thereof is bivalent (e.g., in a “knob in hole” format). A bivalent biparatopic anti-FRα antibody or antigen binding fragment thereof can comprise, for example, two scFvs, two VH-VL pairs on separate polypeptide chains, or one scFv and one VH-VL pair on separate polypeptide chains.

In some aspects, a bivalent biaparatopic anti-FRα antibody or antigen binding fragment thereof comprises an scFv and a VH-VL pair on separate polypeptides. In such aspects, the scFv can be fused to a heavy chain constant region and the VH can be fused to a heavy chain constant region. In some aspects, the constant regions have “knob and hole” sequences. The “knob” sequence can be in the heavy chain constant region fused to the scFv, and the “hole” sequence can be fused to the constant region fused to the VH. Alternatively the “hole” mutation can be in the heavy chain constant region fused to the scFv, and the “knob” sequence can be fused to the constant region fused to the VH.

In some aspects, a biparatopic anti-FRα antibody or antigen binding fragment thereof is trivalent.

In some aspects, a biparatopic anti-FRα antibody or antigen-binding fragment thereof is tetravalent. Tetravalent antibodies and are described, for instance, in M. J. Coloma, S. L. Morrison, Nat. Biotechnol., 15(2):159-63 (1997), which is herein incorporated by reference in its entirety.

In some aspects, a tetravalent biparatopic anti-FRα antibody or antigen binding fragment thereof comprises two FRα-binding domains that are scFvs and two FRα-binding domains that comprises VHs and VLs on separate polypeptides. In such aspects, the scFvs can be fused to the N- or C-terminal of the polypeptide comprising the VH. The scFvs can also be fused to the N- or C-terminal of the polypeptide comprising the VL.

A tetravalent biparatopic anti-FRα antibody or antigen binding fragment thereof can comprise two polypeptides wherein the first polypeptide comprises a heavy chain constant region, a VH, and an scFv and the second polypeptide comprises a light chain constant region and a VL. A tetravalent biparatopic anti-FRα antibody or antigen binding fragment thereof can also comprise two polypeptides wherein the first polypeptide comprises a heavy chain constant region and a VH and the second polypeptide comprises a light chain constant region, a VL, and an scFv.

In some aspects, a biparatopic anti-FRα antibody or antigen binding fragment thereof is a bispecific heterodimeric diabody, e.g., a tetrameric bispecific heterodimeric diabody. As used herein, the term “bispecific heterodimeric diabody” refers to a complex of two or more polypeptide chains or proteins, and each can comprise at least one antibody VL and one antibody VH domain, and wherein the VL and VH domains in each polypeptide chain are from different antibodies.

In some aspects, a biparatopic anti-FRα-antibody or antigen-binding fragment thereof comprise the sequences disclosed in Table 8 below, i.e., polypeptides comprising the amino acid sequences of SEQ ID NOs:61, 62, and 56.

TABLE 8
FR57scFv-knob-Mov19-hole Sequences

| Name | Sequences |
|---|---|
| FR57scFv-Fc- | EIVLTQSPATLSVTPGDRVSLSCRASQNINNNLHWYQQKPGQSPRLLIK |
| knob (C220S, | YVSQSVSGIPDRFSGSGSGTDFTLSISSVEPEDEGMYFCQQSNSWPHYT |
| T366W) | FGCGTKLEIKGGGGSGGGGSGGGGSGGGGSEVQLVQSGGGLVQPGGS |
| | RRLSCAASGFTFSSFGMHWVRQAPGKCLEWVAYISSGSSTISYADSVK |
| | GRFTISRDNSKKTLLLQMTSLRAEDTAMYYCAREAYGSSMEYWGQGT |
| | LVTVSSGSEPKSSDKTHTCPPCPAPELLGGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTL |
| | PPSREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPG |
| | (SEQ ID NO: 61) |
| Mov19-Fc-hole | QVQLVQSGAEVVKPGASVKISCKASGYTFTGYFMNWVKQSPGQSLEW |
| (T366S, L368A, | IGRIHPYDGDTFYNQKFQGKATLTVDKSSNTAHMELLSLTSEDFAVYY |
| Y407V) | CTRYDGSRAMDYWGQGTTVTVSSASTKGPSVFPLAPSSKSTSGGTAAL |
| | GCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEW |
| | ESNGQPENNYKTTPPVLDSDGSFFLVSKLTVDKSRWQQGNVFSCSVM |
| | HEALHNHYTQKSLSLSPG (SEQ ID NO: 62) |
| Mov19-LC | DIVLTQSPLSLAVSLGQPAIISCKASQSVSFAGTSLMHWYHQKPGQQPR |
| | LLIYRASNLEAGVPDRFSGSGSKTDFTLTISPVEAEDAATYYCQQSREY |
| | PYTFGGGTKLEIKRTVAAPSVFIFPPSDEQLKSGTASVVCLLNNFYPREA |
| | KVQWKVDNALQSGNSQESVTEQDSKDSTYSLSSTLTLSKADYEKHKV |
| | YACEVTHQGLSSPVTKSFNRGEC (SEQ ID NO: 56) |

In some aspects, an anti-FRα-antibody or antigen-binding fragment thereof is a murine, chimeric, or humanized anti-FRα-antibody or antigen-binding fragment thereof. As used herein, a humanized anti-FRα-antibody or antigen-binding fragment thereof can be a resurfaced anti-FRα-antibody or antigen-binding fragment thereof.

In some aspects, an anti-FRα-antibody or antigen-binding fragment thereof binds to human FRα but not to FOLR2 or FOLR3.

The affinity or avidity of an antibody for an antigen can be determined experimentally using any suitable method well known in the art, e.g., cytometry (including flow cytometry), enzyme-linked immunoabsorbent assay (ELISA), or radioimmunoassay (RIA), or kinetics (e.g., surface plasmon resonance spectroscopy (BIACORE™) analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kₒₙ, K_{off}) are made with standardized solutions of antibody and antigen, and a standardized buffer, as known in the art and such as the buffer described herein.

In some aspects, an anti-FRα-antibody or antigen-binding fragment thereof comprises a heavy chain constant region, such as an IgG1, IgG2, IgG3, IgG4, IgA, IgE, IgM or IgD constant region. In some aspects, the heavy chain constant region is an IgG1 heavy chain constant region or an IgG4 heavy chain constant region. Furthermore, in some aspects, an anti-FRα-antibody or antigen-binding fragment thereof can comprise a light chain constant region, either a kappa light chain constant region or a lambda light chain constant region. In some aspects, the light chain constant region is a kappa light chain constant region.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies.

Alternatively monoclonal antibodies can also be made using recombinant DNA methods as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some aspects, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In some aspects, the monoclonal antibody against the human FRα is a humanized antibody. In some aspects, such antibodies are used therapeutically to reduce antigenicity and HAMA (human anti-mouse antibody) responses when administered to a human subject.

Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing FRα-binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for the antigen FRα and other favorable biological properties. To achieve this goal, humanized (or human) or engineered anti-FRα antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as FRα. In this way, framework (FR) residues can be selected and combined from the consensus and import sequences so that the desired antibody characteristic, such as increased affinity for the target antigen(s), is achieved.

Humanization, resurfacing or engineering of antibodies disclosed herein can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Roguska et al., Proc. Natl. Acad. Sci., USA, 91(3):969-973 (1994), Roguska et al., Protein Eng. 9(10):895-904 (1996), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538,195; and 7,342,110, each of which is entirely incorporated herein by reference, including the references cited therein.

The agents that specifically bind to FRα disclosed herein also encompass antibody fragments. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In some aspects, antibody fragments are produced recombinantly. Fab, Fv, and scFv antibody fragments can all be expressed in and secreted from E. coli or other host cells, thus allowing the production of large amounts of these fragments. Such antibody fragments can also be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

It should be appreciated that modified antibodies can comprise any type of variable region that provides for the association of the antibody with the polypeptides of a human FRα. In this regard, the variable region can comprise or be derived from any type of mammal that can be induced to mount a humoral response and generate immunoglobulins against the desired tumor associated antigen. As such, the variable region of the modified antibodies can be, for example, of human, murine, non-human primate (e.g., cynomolgus monkeys, macaques, etc.) or lupine origin. In some aspects both the variable and constant regions of the modified immunoglobulins are human. In some aspects the variable regions of compatible antibodies (usually derived from a non-human source) can be engineered or specifically tailored to improve the binding properties or reduce the immunogenicity of the molecule. In this respect, variable regions can be humanized or otherwise altered through the inclusion of imported amino acid sequences.

In some aspects, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in some aspects from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

The polypeptides and analogs can be further modified to contain additional chemical moieties not normally part of the protein. Those derivatized moieties can improve the solubility, the biological half life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, PA (2000).

### II. Soluble FRα and Detection Thereof

Methods of detecting soluble FRα and antibodies that can be used for the same have been disclosed, for example, in WO 2012/061759, WO 2014/036495 and WO 2019/050935, each of which is herein incorporated by reference in its entirety. In addition, kits that can be used to detect soluble FRα (such as Human FOLR1 Quantikine© ELISA Kit (R&D systems)) are commercially available. Soluble FRα can be detected in a sample obtained from a patient, e.g., a patient having cancer. The sample can comprise a bodily fluid. In some aspects, the bodily fluid is plasma, serum, or ascites fluid. In some aspects, the sample is plasma. In some aspects, the sample comprises a peripheral blood sample.

In some aspects, soluble FRα is detected using enzyme-linked immunosorbent assay (ELISA).

Soluble FRα can be detected using anti-FRα antibodies and antigen-binding fragments thereof. Anti-FRα antibodies and antigen-binding fragments thereof useful in the detection of soluble FRα can be called soluble FRα-detection antibodies or antigen-binding fragments thereof. Soluble FRα-detection antibodies or antigen-binding fragments thereof include, e.g., the muFR1-9 and muFR1-13 antibodies described in Section II, above. Soluble FRα-detection antibodies or antigen-binding fragments thereof also include, e.g., antibodies and antigen-binding fragments thereof that comprise the six CDRs (i.e., the VH-CDR1, VH-CDR2, VH-CDR3, VL-CDR1, VL-CDR2, and VL-CDR3) of muFR1-9 and muFR1-13, or the VH and/or VL of muFR1-9 and muFR1-13.

In some aspects, a soluble FRα-detection antibody or antigen-binding fragment thereof comprises (a) VH CDR1, VH CDR2, and VH CDR3 comprising the amino acid sequences of SEQ ID NOs:18-20, respectively; and (b) VL CDR1, VL CDR2, and VL CDR3 comprising the amino acid sequences of SEQ ID NOs:21-23, respectively.

In some aspects, a soluble FRα-detection antibody or antigen-binding fragment thereof comprises (a) VH CDR1, VH CDR2, and VH CDR3 comprising the amino acid sequences of SEQ ID NOs:24-26, respectively; and (b) VL CDR1, VL CDR2, and VL CDR3 comprising the amino acid sequences of SEQ ID NOs:27-29, respectively.

In some aspects, a soluble FRα-detection antibody or antigen-binding fragment thereof comprises (a) a variable heavy chain (VH) comprising the amino acid sequence of SEQ ID NO:39 and/or (b) a variable light chain (VL) comprising the amino acid sequence of SEQ ID NO:45.

In some aspects, a soluble FRα-detection antibody or antigen-binding fragment thereof comprises (a) a variable heavy chain (VH) comprising the amino acid sequence of SEQ ID NO:40 and/or (b) a variable light chain (VL) comprising the amino acid sequence of SEQ ID NO:46.

In some aspects, a soluble FRα-detection antibody or antigen-binding fragment thereof comprises (a) a heavy chain (HC) comprising the amino acid sequence of SEQ ID NO:51 and/or (b) a light chain (CL) comprising the amino acid sequence of SEQ ID NO:57.

In some aspects, a soluble FRα-detection antibody or antigen-binding fragment thereof comprises (a) a heavy chain (HC) comprising the amino acid sequence of SEQ ID NO:52 and/or (b) a light chain (CL) comprising the amino acid sequence of SEQ ID NO:58.

In some aspects, binding of huMov19 to FRα does not competitively inhibit the binding of an soluble FRα-detection antibody or antigen-binding fragment to FRα. In some aspects, binding of IMGN853 to FRα does not competitively inhibit the binding of an soluble FRα-detection antibody or antigen-binding fragment to FRα. In some aspects binding of an antibody or antigen-binding fragment comprising (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774 to FRα does not competitively inhibit the binding of an soluble FRα-detection antibody or antigen-binding fragment to FRα.

In some aspects, binding of folic acid to FRα does not competitively inhibit the binding of a soluble FRα-detection antibody or antigen-binding fragment to FRα.

In some aspects, a soluble FRα-detection antibody or antigen-binding fragment binds to human FRα with a Kd of about 1.0 nM to about 10 nM. In some aspects, a soluble FRα-detection antibody or antigen-binding fragment binds to human FRα with a Kd of about 0.5 nM to about 5 nM.

In some aspects, soluble FRα is detecting using a method comprising liquid chromatography-mass spectrometry (LC/MS), enzyme-linked immunosorbent assay (ELISA), or electrochemiluminescence immunoassay (ECLIA) or meso scale discovery (MSD), which is a method similar to ELISA except MSD uses electrochemiluminescence (ECL) as a detection technique as opposed to a colormetric reaction employed by ELISA. In some aspects, soluble FRα is detecting using a method comprising ELISA.

In some aspects, soluble FRα is detecting using electrochemiluminescence (ECL). In some aspects, soluble FRα is detecting using a colormetric reaction.

In some aspects, soluble FRα is detecting using a multi-step approach. In one such multi-step approach, an initial immunocapture step is performed to enrich for FRα in a sample, followed by digestion of the FRα into peptides and analysis by liquid chromatography-mass spectrometry (LC/MS). Analysis of the peptides by LC/MS further allows the level of FRα present in a sample to be quantitatively determined, including, for example, the level of FRα in a sample from a patient with cancer. In some aspects, the method of detecting human FRα in a sample comprises: (a) capturing said FRα with an immunocapture reagent bound to a solid support; (b) eluting FRα from the solid support; (c) digesting the eluted FRα; and (d) performing liquid chromatography-mass spectrometry (LC/MS) analysis on the digested FRα, wherein the FRα is detected by monitoring the chromatographic separation and mass spectrometric response of at least one signature FRα peptide. Such methods provide the advantage of enhanced sensitivity and selectivity.

An initial immunocapture step is performed on the sample using an immunocapture reagent bound to a solid support. The immunocapture reagent can be any immunological reagent which binds to FRα, including, for example, a soluble FRα-detection antibody or antigen-binding fragment thereof as discussed above. When an antibody or antigen-binding fragment is used as an immunocapture reagent, the binding of the antibody or antigen-binding fragment to FRα may not be competitively inhibited by binding of an antibody-based active agent, such as IMGN853, to FRα in the sample. Moreover, when an antibody or antigen-binding fragment is used as an immunocapture reagent, the binding of the antibody or antigen-binding fragment to FRα may not be inhibited by folic acid present in the sample.

In some aspects, the immunocapture reagent is biotinylated. In some aspects, the immunocapture reagent is bound to the solid support through a biotin-streptavidin interaction. In some aspects, the solid support is a mass spectrometric immunoassay (MSIA) microcolumn. In some aspects, the immunocapture reagent comprises magnetic beads.

To perform the initial immunocapture step, a sample containing FRα can be incubated with the immunocapture reagent bound to a solid support. A wash step can be performed after the immunocapture step to further purify the captured FRα. In some aspects, one or more wash steps are performed following incubation of the sample with the immunocapture reagent and prior to elution of the captured FRα. In some aspects, two or more wash steps are performed prior to elution of the captured FRα. In some aspects, at least two, at least three, at least four, at least five, at least six, at least seven, at least eight, at least nine, or at least ten wash steps are performed on the captured FRα. In some aspects, the wash step comprises contacting the captured FRα with washing buffers. In some aspects, the washing buffers are commercially available washing buffers. In some aspects, the wash step comprises contacting the captured FRα with a salt solution and a detergent. In some aspects, the salt solution is 400 mM NaCl and the detergent is 0.1% Tween 20.

Following immunocapture, the FRα can be released from the solid support by performing an elution step. In some aspects, the elution step is performed by contacting the captured FRα with an acidic solution. In some aspects, the acidic solution is a commercially available solution. In some aspects, the eluate is brought to neutral pH by addition of a neutralization buffer. In some aspects, the neutralization buffer is 500 mM Ammonium Bicarbonate at pH 8. In some aspects, the neutralization buffer is a commercially available buffer.

Following immunocapture and elution, the resulting FRα protein can be digested into peptides and analyzed by liquid chromatography-mass spectrometry (LC/MS). In some aspects, the FRα-containing solution is alkylated and reduced prior to analysis by LC/MS. In some aspects, the FRα is alkylated with methanol. In some aspects, the FRα is reduced by contacting the FRα with a solution containing tris(2-carboxyethyl)phosphine (TCEP). In some aspects, a solution containing 100 mM TCEP is used to reduce the FRα. In some aspects, a cysteine blocking reagent is added to the FRα-containing solution after alkylation and reduction. In some aspects, the cysteine blocking reagent is iodoacetamide (IAM). In some aspects, a solution containing 100 mM IAM is added to the FRα-containing solution.

After alkylation and reduction, the FRα is digested into peptides. In some aspects, the FRα is digested with trypsin. In some aspects, the FRα is digested with Lys-C. In some aspects, the FRα is digested with a mixture of trypsin and Lys-C. In some aspects, the FRα is digested by contacting the FRα with a 50 mM ammonium bicarbonate solution containing 30 ng/μL trypsin/Lys-C.

Digestion of the FRα with trypsin/Lys-C produces peptides that are useful in conducting quantitative analysis of the sample by LC/MS. Exemplary signature peptides produced by digestion of FRα are provided in Table 9 below.

TABLE 9
FRα signature peptide amino acid sequences

| | |
|---|---|
| sFRα Signature Peptide Sequence 1 | TELLNVCMNAK (SEQ ID NO: 68) |
| sFRα Signature Peptide Sequence 2 | IAWAR (SEQ ID NO: 69) |
| sFRα Signature Peptide Sequence 3 | VLNVPLCK (SEQ ID NO: 70) |
| sFRα Signature Peptide Sequence 4 | CIQMWFDPAQGNPNEEVAR (SEQ ID NO: 71) |

After digestion of FRα into peptides, the peptide-containing solution can be prepared for LC/MS analysis. In some aspects, a surfactant is added to the peptide-containing solution prior to LC/MS analysis. In some aspects, the surfactant is a commercial reagent formulated for mass spectrometry analysis. In some aspects, the reaction with surfactant is quenched by adding 10% formic acid.

Following digestion and preparation of the samples for LC/MS analysis, the samples can be injected into an LC/MS instrument and analyzed. In some aspects, at least two signature peptides of FRα are selected and monitored at the LC/MS analysis step. In some aspects, at least three signature peptides of FRα are selected and monitored at the LC/MS analysis step. In some aspects, at least four signature peptides of FRα are selected and monitored at the LC/MS analysis step. In some aspects, the at least four signature peptides selected and monitored at the LC/MS step comprise: a peptide having the amino acid sequence of SEQ ID NO:68; a peptide having the amino acid sequence of SEQ ID NO:69; a peptide having the amino acid sequence of SEQ ID NO:70; and a peptide having the amino acid sequence of SEQ ID NO:71. In some aspects, quantitative measurements of the FRα levels are provided by LC/MS analysis. In some aspects, the level of FRα in the sample is quantitated by comparing the level of FRα in the sample to a reference level of FRα. Methods of analysis by LC/MS analysis are well known in the art and are described in, e.g., Yang et al. Scientific Reports. 2015 Nov. 17; 5:16733; doi10.1038/srep16733.

In some aspects provided herein, soluble FRα is detected using a method that can detect levels FRα at least as low as 0.5 ng/mL FRα in a sample. In some aspects provided herein, soluble FRα is detected using a method that can detect levels FRα at least as low as 0.3 ng/mL FRα in a sample. In some aspects provided herein, soluble FRα is detected using a method that can detect levels FRα at least as low as 0.25 ng/mL FRα in a sample. In some aspects provided herein, soluble FRα is detected using a method that can detect levels FRα at least as low as 0.2 ng/mL FRα in a sample. In some aspects provided herein, soluble FRα is detected using a method that can detect levels FRα at least as low as 0.15 ng/mL FRα in a sample.

In some aspects provided herein, soluble FRα is detected using a method wherein a signal-to-noise ratio of at least 5 is observed. In some aspects provided herein, soluble FRα is detected using a method wherein a signal-to-noise ratio of at least 6 is observed. In some aspects provided herein, soluble FRα is detected using a method wherein a signal-to-noise ratio of at least 7 is observed. In some aspects provided herein, soluble FRα is detected using a method wherein a signal-to-noise ratio of at least 8 is observed. In some aspects provided herein, soluble FRα is detected using a method wherein a signal-to-noise ratio of at least 9 is observed. In some aspects provided herein, soluble FRα is detected using a method wherein a signal-to-noise ratio of at least 10 is observed.

In some aspects, a soluble FRα level is a soluble FRα level which is normalized to the size of a tumor; such a normalized soluble FRα level can be determined by dividing a soluble FRα level by the size of the tumor. In some aspects, a soluble FRα level is not normalized to the size of a tumor.

### IV. Soluble FRα as an Indicator of Efficacy of FRα-Targeting Therapies

As described herein, higher soluble FRα levels in a patient with cancer are surprisingly associated with improved responses to FRα-targeting therapies (e.g., anti-FRα immunoconjugates), and this association can be independent of membrane FRα levels as measured by immunohistochemistry (IHC). Thus, a soluble FRα level in a patient that is equal to or greater than a target soluble FRα level can be an independent predictor of the responsiveness of a cancer in the patient to an anti-FRα active agent (e.g., anti-FRα immunoconjugate such as IMGN853 or IMGN151). Accordingly, provided herein are methods of treating cancer in a patient comprising administering a pharmaceutical composition comprising an anti-FRα active agent (e.g., anti-FRα immunoconjugate such as IMGN853 or IMGN151) to a cancer patient with a soluble FRα level equal to or greater than a target soluble FRα level. In some aspects, the patient's soluble FRα has been detected prior to the administration (e.g., as described in Section III, above). In some aspects, the method further comprises detecting the patient's soluble FRα prior to the administration (e.g., as described in Section III, above).

In some aspects, a method of increasing the efficacy of cancer therapy comprises administering a pharmaceutical composition comprising an anti-FRα active agent (e.g., anti-FRα immunoconjugate such as IMGN853 or IMGN151) to a patient with cancer, wherein the patient has a soluble FRα level equal to or greater than a target soluble FRα level. In some aspects, the patient's soluble FRα has been detected prior to the administration (e.g., as described in Section III, above). In some aspects, the method further comprises detecting the patient's soluble FRα prior to the administration (e.g., as described in Section III, above).

In some aspects, a method of treating cancer in a patient comprises determining if the patient has a soluble FRα level equal to or greater than a target soluble FRα level (e.g., by obtaining soluble FRα test results measured by another party) and administering a pharmaceutical composition comprising an anti-FRα active agent (e.g., anti-FRα immunoconjugate such as IMGN853 or IMGN151) if a soluble FRα level equal to or greater than a target soluble FRα level has been detected in the patient.

In some aspects, a method of treating cancer in a patient comprises determining if a soluble FRα level equal to or greater than a target soluble FRα level is present in a sample obtained from a patient with cancer and instructing a physician to administer a pharmaceutical composition comprising an anti-FRα active agent (e.g., anti-FRα immunoconjugate such as IMGN853 or IMGN151) if a soluble FRα level equal to or greater than a target soluble FRα level has been detected in the sample.

In some aspects, a method of treating cancer in a patient comprises (i) administering a pharmaceutical composition comprising an anti-FRα active agent to the patient if the patient has a soluble FRα level equal to or greater than a target soluble FRα level; and (ii) administering chemotherapy to the patient if the patient does not have a soluble FRα level equal to or greater than a target soluble FRα level or a cancer sample obtained from the patient does not have a high FRα IHC score. In some aspects, the cancer sample used for the IHC determination was obtained at least 6 months, at least 9 months, at least a year, or at least 18 months prior to detection of the patient's soluble FRα level. In some aspects, the patient's soluble FRα has been detected prior to the administration (e.g., as described in Section III, above). In some aspects, the method further comprises detecting the patient's soluble FRα prior to the administration (e.g., as described in Section III, above).

In some aspects, the FRα IHC score has been detected prior to the administration. In some aspects the cancer sample used for the IHC determination was obtained at least 6 months, at least 9 months, at least a year, at least 18 months, or at least 2 years prior to the administration. In some aspects, the method further comprises detecting the FRα IHC score prior to the administration. In some aspects, the soluble FRα and the FRα IHC score have been detected prior to the administration. In some aspects the cancer sample used for the IHC determination was obtained at least 6 months, at least 9 months, at least a year, at least 18 months, or at least 2 years prior to the administration. In some aspects, the method further comprises detecting the soluble FRα and the FRα IHC score prior to the administration. In some aspects, the soluble FRα has been detected prior to the administration, and the method further comprises detecting the FRα IHC score prior to the administration. In some aspects, the FRα IHC score has been detected prior to the administration (e.g., in a cancer sample obtained at least 6 months, at least 9 months, at least a year, at least 18 months, or at least 2 years prior to the administration), and the method further detecting the soluble FRα prior to the administration.

In some aspects, a method for identifying a cancer in a patient as likely to respond to an anti-FRα active agent comprises assaying for soluble FRα in a sample obtained from the patient and optionally determining the FRα IHC score in a sample obtained from the patient, wherein the presence of a soluble FRα level equal to or greater than a target soluble FRα level and/or a high FRα IHC score indicates the cancer is likely to respond to the anti-FRα active agent, optionally wherein the method further comprises administering a pharmaceutical composition comprising the anti-FRα active agent to the patient if the cancer is likely to respond.

Effective target soluble FRα levels for determining the likelihood of a cancer to respond to an anti-FRα active agent are demonstrated here. In some aspects, the target soluble FRα level is about 0.5 ng/mL, about 0.6 ng/mL, about 0.7 ng/mL, about 0.75 ng/mL, about 0.8 ng/mL, about 0.9 ng/mL, or about 1 ng/mL. In some aspects, the target soluble FRα level is about 1.1 ng/mL, about 1.2 ng/mL, about 1.25 ng/mL, about 1.3 ng/mL, about 1.4 ng/mL, about 1.5 ng/mL, about 1.6 ng/mL, about 1.7 ng/mL, about 1.75 ng/mL, about 1.8 ng/mL, about 1.9 ng/mL, or about 2 ng/mL. In some aspects, the target soluble FRα level is about 2.1 ng/mL, about 2.2 ng/mL, about 2.25 ng/mL, about 2.3 ng/mL, about 2.4 ng/mL, about 2.5 ng/mL, about 2.6 ng/mL, about 2.7 ng/mL, about 2.75 ng/mL, about 2.8 ng/mL, about 2.9 ng/mL, or about 3 ng/mL. In some aspects, the target soluble FRα level is about 3.1 ng/mL, about 3.2 ng/mL, about 3.25 ng/mL, about 3.3 ng/mL, about 3.4 ng/mL, about 3.5 ng/mL, about 3.6 ng/mL, about 3.7 ng/mL, about 3.75 ng/mL, about 3.8 ng/mL, about 3.9 ng/mL, or about 4 ng/mL. In some aspects, the target soluble FRα level is about 4.1 ng/mL, about 4.2 ng/mL, about 4.25 ng/mL, about 4.3 ng/mL, about 4.4 ng/mL, about 4.5 ng/mL, about 4.6 ng/mL, about 4.7 ng/mL, about 4.75 ng/mL, about 4.8 ng/mL, about 4.9 ng/mL, or about 5.0 ng/mL.

As demonstrated herein, patients with ovarian, primary peritoneal, or fallopian tube cancer and an above average level of soluble FRα for such patients have an increased likelihood of responding to an anti-FRα active agent. Accordingly, in some aspects, a high level of soluble FRα refers to a level that is above average for patients with ovarian, primary peritoneal, or fallopian tube cancer. In some aspects, a high level of soluble FRα refers to a level that is greater than the level in 75% of patients with ovarian, primary peritoneal, or fallopian tube cancer. In some aspects, a high level of soluble FRα refers to a level that is above average for patients with ovarian, primary peritoneal, or fallopian tube cancer, who also have a tumor with medium (50-74% cells positive) or high (at least 75% cells positive) membrane FRα levels as determined by PS2 scoring. In some aspects, a high level of soluble FRα refers to a level that is greater than the level in 75% of patients with ovarian, primary peritoneal, or fallopian tube cancer, who also have a tumor with medium (50-74% cells positive) or high (at least 75% cells positive) membrane FRα levels as determined by PS2 scoring.

### V. Anti-FRα Active Agents

As provided herein, anti-FRα active agent can be administered to patients with soluble FRα. Anti-FRα active agents include, for example, anti-FRα antibodies and antigen-binding fragments thereof as well as anti-FRα immunoconjugates. Anti-FRα active agents and methods of using the same have been described, for example, in WO 2011/106528, WO 2015/054400, and U.S. Published Application No. 2020/0362029, each of which is herein incorporated by reference in its entirety. Additional anti-FRα antibodies and antigen binding fragments thereof are known in the art and have been disclosed, for example, in WO 2012/061759, U.S. Published Application No. 2019/0233512, U.S. Published Application No. 2020/0147229, U.S. Published Application No. 2020/0297860 U.S. Published Application No. 2020/0353076, U.S. Pat. Nos. 10,822,410 and 10,101,343, each of which is herein incorporated by reference in its entirety Anti-FRα active agents include, e.g., the FR57 antibody and variants thereof and the huMov19 antibody described in Section II, above as well as antigen-binding fragments thereof and immunoconjugates thereof.

Anti-FRα active agents also include, e.g., antibodies and antigen-binding fragments thereof that comprise the six CDRs (i.e., the VH-CDR1, VH-CDR2, VH-CDR3, VL-CDR1, VL-CDR2, and VL-CDR3) of FR57, variants thereof, and huMov19 and immunoconjugate thereof. Anti-FRα active agents also include, e.g., antibodies and antigen-binding fragments thereof that comprise the six VH and/or VL of FR57, variants thereof, and huMov19, as well immunoconjugates thereof.

In some aspects, an anti-FRα-antibody or antigen-binding fragment thereof in an anti-FRα active agent (e.g., an anti-FRα immunoconjugate) comprises an anti-FRα antibody or antigen-biding fragment thereof that binds to the same FRα epitope as an antibody comprising the VH of SEQ ID NO:38 and a VL of SEQ ID NO:44 and/or competitively inhibits binding of an antibody comprising the VH of SEQ ID NO:38 and a VL of SEQ ID NO:44 to FRα.

In some aspects, an anti-FRα-antibody or antigen-binding fragment thereof in an anti-FRα active agent (e.g., an anti-FRα immunoconjugate) comprises an anti-FRα antibody or antigen-biding fragment thereof that binds to the same FRα epitope as an antibody comprising the VH of SEQ ID NO:37 and a VL of SEQ ID NO:43 and/or competitively inhibits binding of an antibody comprising the VH of SEQ ID NO:37 and a VL of SEQ ID NO:43 to FRα.

In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:2-4, respectively; and (b) VL CDR1, VL CDR2, and VL CDR3 comprising the amino acid sequences of SEQ ID NOs:7-9, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:5, 6, and 4, respectively; and (b) VL CDR1, VL CDR2, and VL CDR3 comprising the amino acid sequences of SEQ ID NOs:7-9, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:2, 6, and 4, respectively; and (b) VL CDR1, VL CDR2, and VL CDR3 comprising the amino acid sequences of SEQ ID NOs:7-9, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:5, 3, and 4, respectively; and (b) VL CDR1, VL CDR2, and VL CDR3 comprising the amino acid sequences of SEQ ID NOs:7-9, respectively.

In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:10-12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:13, 14, and 12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:10, 14, and 12, respectively; and (b) VL CDR1, VL CDR2, and VL CDR3 comprising the amino acid sequences of SEQ ID NOs:15-17, respectively. In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:13, 11, and 12, respectively; and (b) VL CDR1, VL CDR2, and VL CDR3 comprising the amino acid sequences of SEQ ID NOs:15-17, respectively.

In some aspects, an anti-FRα-antibody or antigen-binding fragment thereof in a biparatopic anti-FRα active agent (e.g., a biparatopic anti-FRα immunoconjugate) comprises (a) VH CDR1, VH CDR2, and VH CDR3 comprising the amino acid sequences of SEQ ID NOs:2-4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10-12, respectively; and (d) VL CDR1, VL CDR2, and VL CDR3 comprising the amino acid sequences of SEQ ID NOs:15-17, respectively.

In some aspects, an anti-FRα-antibody or antigen-binding fragment thereof in a biparatopic anti-FRα active agent (e.g., a biparatopic anti-FRα immunoconjugate) comprises (a) VH CDR1, VH CDR2, and VH CDR3 comprising the amino acid sequences of SEQ ID NOs:5, 6, and 4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10, 11, and 12, respectively; and (d) VL CDR1, VL CDR2, and VL CDR3 comprising the amino acid sequences of SEQ ID NOs:15-17, respectively.

In some aspects, an anti-FRα-antibody or antigen-binding fragment thereof in a biparatopic anti-FRα active agent (e.g., a biparatopic anti-FRα immunoconjugate) comprises (a) VH CDR1, VH CDR2, and VH CDR3 comprising the amino acid sequences of SEQ ID NOs:2, 6, and 4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10, 14, and 12, respectively; and (d) VL CDR1, VL CDR2, and VL CDR3 comprising the amino acid sequences of SEQ ID NOs:15-17, respectively.

In some aspects, an anti-FRα-antibody or antigen-binding fragment thereof in an anti-FRα active agent (e.g., an anti-FRα immunoconjugate) comprises (a) a variable heavy chain (VH) comprising the amino acid sequence of SEQ ID NO:36, (b) a variable light chain (VL) comprising the amino acid sequence of SEQ ID NO:42.

In some aspects, an anti-FRα-antibody or antigen-binding fragment thereof in an anti-FRα active agent (e.g., an anti-FRα immunoconjugate) comprises (a) a variable heavy chain (VH) comprising the amino acid sequence of SEQ ID NO:37, (b) a variable light chain (VL) comprising the amino acid sequence of SEQ ID NO:43.

In some aspects, an anti-FRα-antibody or antigen-binding fragment thereof in an anti-FRα active agent (e.g., an anti-FRα immunoconjugate) comprises (a) a variable heavy chain (VH) comprising the amino acid sequence of SEQ ID NO:38, (b) a variable light chain (VL) comprising the amino acid sequence of SEQ ID NO:44.

In some aspects, an anti-FRα-antibody or antigen-binding fragment thereof in a biparatopic anti-FRα active agent (e.g., a biparatopic anti-FRα immunoconjugate) comprises (a) a variable heavy chain (VH) comprising the amino acid sequence of SEQ ID NO:36, (b) a variable light chain (VL) comprising the amino acid sequence of SEQ ID NO:42, (c) a VH comprising the amino acid sequence of SEQ ID NO:38 and (d) a VL comprising the amino acid sequence of SEQ ID NO:44.

In some aspects, an anti-FRα-antibody or antigen-binding fragment thereof in a biparatopic anti-FRα active agent (e.g., a biparatopic anti-FRα immunoconjugate) comprises (a) a variable heavy chain (VH) comprising the amino acid sequence of SEQ ID NO:37, (b) a variable light chain (VL) comprising the amino acid sequence of SEQ ID NO:43, (c) a VH comprising the amino acid sequence of SEQ ID NO:38 and (d) a VL comprising the amino acid sequence of SEQ ID NO:44.

In some aspects, an anti-FRα-antibody or antigen-binding fragment thereof in a biparatopic anti-FRα active agent (e.g., a biparatopic anti-FRα immunoconjugate) comprises (a) single chain variable region (scFv) comprising the amino acid sequence of SEQ ID NO:60, (b) a VH comprising the amino acid sequence of SEQ ID NO:38 and (c) a VL comprising the amino acid sequence of SEQ ID NO:44.

In some aspects, an anti-FRα-antibody or antigen-binding fragment thereof in an anti-FRα active agent (e.g., an anti-FRα immunoconjugate) comprises (a) a heavy chain (HC) comprising the amino acid sequence of SEQ ID NO:48 and/or (b) a light chain (CL) comprising the amino acid sequence of SEQ ID NO:54.

In some aspects, an anti-FRα-antibody or antigen-binding fragment thereof in an anti-FRα active agent (e.g., an anti-FRα immunoconjugate) comprises (a) a heavy chain (HC) comprising the amino acid sequence of SEQ ID NO:49 and/or (b) a light chain (CL) comprising the amino acid sequence of SEQ ID NO:55.

In some aspects, an anti-FRα-antibody or antigen-binding fragment thereof in an anti-FRα active agent (e.g., an anti-FRα immunoconjugate) comprises (a) a heavy chain (HC) comprising the amino acid sequence of SEQ ID NO:50 and/or (b) a light chain (CL) comprising the amino acid sequence of SEQ ID NO:56.

In some aspects, an anti-FRα-antibody or antigen-binding fragment thereof in a biparatopic anti-FRα active agent (e.g., a biparatopic anti-FRα immunoconjugate) comprises (a) a polypeptide comprising the amino acid sequence of SEQ ID NO:61; (b) a polypeptide comprising the amino acid sequence of SEQ ID NO:62; and/or (c) a polypeptide comprising the amino acid sequence of SEQ ID NO:56.

In some aspects, an anti-FRα-antibody or antigen-binding fragment thereof in an anti-FRα active agent (e.g., an anti-FRα antibody or immunoconjugate) comprises (a) VH CDR1, VH CDR2, and VH CDR3 comprising the amino acid sequences of SEQ ID NOs:80-82, respectively and (b) VL CDR1, VL CDR2, and VL CDR3 comprising the amino acid sequences of SEQ ID NOs:86-88, respectively.

In some aspects, an anti-FRα-antibody or antigen-binding fragment thereof in an anti-FRα active agent (e.g., an anti-FRα antibody or immunoconjugate) comprises (a) VH CDR1, VH CDR2, and VH CDR3 comprising the amino acid sequences of SEQ ID NOs:83-85, respectively and (b) VL CDR1, VL CDR2, and VL CDR3 comprising the amino acid sequences of SEQ ID NOs:89, 90, and 88, respectively.

In some aspects, an anti-FRα-antibody or antigen-binding fragment thereof in an anti-FRα active agent (e.g., an anti-FRα antibody or immunoconjugate) comprises a VH comprising the amino acid sequence of SEQ ID NO:99 and a VL comprising the amino acid sequences of SEQ ID NO:101.

In some aspects, an anti-FRα-antibody or antigen-binding fragment thereof in an anti-FRα active agent (e.g., an anti-FRα antibody or immunoconjugate) comprises a heavy chain comprising the amino acid sequence of SEQ ID NO:103 and a light chain comprising the amino acid sequences of SEQ ID NO:104.

In some aspects, an antibody or antigen-binding fragment thereof in an anti-FRα active agent is farletuzumab (MORAb-003). MORAb-003 is disclosed in U.S. Published Application No. 2020/0297860, which is herein incorporated by reference in its entirety.

In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:91-93, respectively and (b) VL CDR1, VL CDR2, and VL CDR3 comprising the amino acid sequences of SEQ ID NOs:96-98, respectively.

In some aspects, an anti-FRα-antibody or antigen-binding fragment thereof in an anti-FRα active agent (e.g., an anti-FRα immunoconjugate) comprises (a) VH CDR1, VH CDR2, and VH CDR3 comprising the amino acid sequences of SEQ ID NOs:94, 95, and 93, respectively and (b) VL CDR1, VL CDR2, and VL CDR3 comprising the amino acid sequences of SEQ ID NOs:96-98, respectively.

In some aspects, an anti-FRα-antibody or antigen-binding fragment thereof in an anti-FRα active agent (e.g., an anti-FRα immunoconjugate) comprises a VH comprising the amino acid sequence of SEQ ID NO:100 and a VL comprising the amino acid sequences of SEQ ID NO:102. In some aspects, such an a antibody or antigen-binding fragment thereof comprises a non-natural amino acid at heavy chain position F404 according to the Kabat or EU numbering scheme of Kabat. In some aspects, such an a antibody or antigen-binding fragment thereof comprises a non-natural amino acid at heavy chain position Y180 according to the Kabat or EU numbering scheme of Kabat. In some aspects, such an a antibody or antigen-binding fragment thereof comprises a non-natural amino acid at heavy chain position F404 and Y180 according to the Kabat or EU numbering scheme of Kabat. The non-natural amino acid sequence be, e.g., para-azidomethylphenylalanine and p-azido-methyl-L-phenylalanine. The antibody can be, for example, an IgG1 antibody.

In some aspects, an antibody or antigen-binding fragment thereof in an anti-FRα active agent is 1848-H01. 1848-H01 is disclosed in U.S. Published Application No. 2019/0083641, which is herein incorporated by reference in its entirety.

In some aspects, an anti-FRα antibody or antigen-binding fragment in an anti-FRα active agent (e.g., an anti-FRα immunoconjugate) comprises an altered (e.g., mutated or engineered) Fc region. For example, in some aspects, the Fc region has been altered to reduce or enhance the effector functions of the antibody, alter serum half-life or other functional properties of the antibody. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Anti-FRα antibodies or antigen-binding fragments in anti-FRα active agents (e.g., an anti-FRα immunoconjugates) possessing such conferred or altered effector function activity are useful for the treatment and/or prevention of a disease, disorder or infection in which an enhanced efficacy of effector function activity is desired. In some aspects, the Fc region is an isotype selected from IgM, IgA, IgG, IgE, or other isotype.

Although the Fc Region of the anti-FRα antibodies or antigen-binding fragments in anti-FRα active agents (e.g., an anti-FRα immunoconjugates) can possess the ability to bind to one or more Fc receptors (e.g., FcγR(s)), in some aspects the antibody or antigen-binding fragment comprises a variant Fc region having an altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Region), e.g., will have enhanced binding to an activating receptor and/or will have substantially reduced or no ability to bind to inhibitory receptor(s). Thus, the Fc region of the anti-FRα antibodies or antigen-binding fragments in anti-FRα active agents (e.g., an anti-FRα immunoconjugates) can include some or all of the CH2 domain and/or some or all of the CH3 domain of a complete Fc region, or may comprise a variant CH2 and/or a variant CH3 sequence (that may include, for example, one or more insertions and/or one or more deletions with respect to the CH2 or CH3 domains of a complete Fc Region). Such Fc regions may comprise non-Fc polypeptide portions, or may comprise portions of non-naturally complete Fc regions, or may comprise non-naturally occurring orientations of CH2 and/or CH3 domains (such as, for example, two CH2 domains or two CH3 domains, or in the N-terminal to C-terminal direction, a CH3 domain linked to a CH2 domain, etc.).

Fe Region modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, et al., Cancer Res. 57(18):8882-8890 (2007)). Table 10 lists exemplary single, double, triple, quadruple and quintuple substitutions (numbering is that of the EU index as in Kabat, and substitutions are relative to the amino acid sequence of SEQ ID NO:72) of exemplary modification that increase binding to activating receptors and/or reduce binding to inhibitory receptors.

TABLE 10
Variations of Activating Fc Regions
Single-Site Variations

| | | | |
|---|---|---|---|
| F243L | R292G | D270E | R292P |
| Y300L | P396L | | |

Double-Site Variations

| | | | |
|---|---|---|---|
| F243L and R292P | F243L and Y300L | F243L and P396L | R292P and Y300L |
| D270E and P396L | R292P and V305I | P396L and Q419H | P247L and N421K |
| R292P and P396L | Y300L and P396L | R255L and P396L | R292P and P305I |
| K392T and P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L and P396L | F243L, R292P, V305I, Y300L and P396L |
| L235P, F243L, R292P, Y300L and P396L | |

Exemplary variants of human IgG1 Fe Regions with reduced binding to CD32B and/or increased binding to CD16A contain F243L, R292P, Y300L, V3051, or P396L substitutions, wherein the numbering is that of the EU index as in Kabat. These amino acid substitutions may be present in a human IgG1 Fc Region in any combination. In some aspects, the variant human IgG1 Fc Region contains a F243L, R292P and Y300L substitution. In some aspects, the variant human IgG1 Fc Region contains a F243L, R292P, Y300L, V3051, and P396L substitution.

In some aspects, the anti-FRα antibody or antigen-binding fragment in an anti-FRα active agent (e.g., an anti-FRα immunoconjugate) comprises an immunoglobulin heavy chain constant region containing a modification that decreases effector function (see, e.g., Idusogie et al., J. Immunol. 166:2571-2575 (2001); Sazinsky et al., PNAS USA 105:20167-20172 (2008); Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-26 (2000); Cole et al., Transplantation 68:563-571 (1999); Hutchins et al., PNAS USA 92:11980-11984 (1995); Reddy et al., J. Immunol. 164:1925-1933 (2000); WO97/11971, and WO07/106585; U.S. Appl. Publ. 2007/0148167A1; McEarchern et al., Blood 109:1185-1192 (2007); Strohl, Curr. Op. Biotechnol. 20:685-691 (2009); and Kumagai et al., J. Clin. Pharmacol. 47:1489-1497 (2007), the contents of each of which is herein incorporated by reference in its entirety).

In some aspects, the Fc region of the anti-FRα antibody or antigen-binding fragment in an anti-FRα active agent (e.g., an anti-FRα immunoconjugate) exhibits decreased (or substantially no) binding to an effector receptor selected from the group consisting of: FcγRIA (CD64), FcγRIIA (CD32A)(allotypes R₁₃₁ and H131), FcγRIIB (CD32B), FcγRIIIA (CD16a) (allotype V158 and F158) and FcγRIIIB (CD16b)(allotype FcγIIIb-NA1 and FcγIIIb-NA2); relative to the binding exhibited by the wild-type IgG Fc Region. In some aspects, the anti-FRα antibody or antigen-binding fragment in an anti-FRα active agent (e.g., an anti-FRα immunoconjugate) Fc region variant effector receptor binding affinity has been reduced to 1/10 or less, 1/50 or less, or 1/100 or less as, compared to the binding affinity of the corresponding antibody or antibody binding fragment comprising the wildtype Fc region of the corresponding immunoglobulin.

In some aspects, the anti-FRα antibody or antigen-binding fragment in an anti-FRα active agent (e.g., an anti-FRα immunoconjugate) comprises an IgG Fc region that exhibits reduced effector function (e.g., reduced ADCC) and comprise a modification at one or more amino acid positions selected from the group consisting of 233, 234, 235, 236, 237, 238, 239, 265, 266, 267, 269, 270, 271, 295, 296, 297, 298, 300, 324, 325, 327, 328, 329, 331, and 332, wherein the amino acid position numbering is according to the EU index as set forth in Kabat. In some aspects, the CH2-CH3 domain of the anti-FRα antibody or antigen-binding fragment in an anti-FRα active agent (e.g., an anti-FRα immunoconjugate) includes any 1, 2, 3, or 4 of the substitutions: L234A, L235A, D265A, N297Q, N297A, and N297G, wherein the numbering is that of the EU index as in Kabat. In some aspects, the CH2-CH3 domains contain an N297Q substitution, an N297A substitution, or L234A and L235A substitutions, as these mutations abolish FcR binding. Alternatively, the anti-FRα antibody or antigen-binding fragment in an anti-FRα active agent (e.g., an anti-FRα immunoconjugate) comprises a CH2-CH3 domain of a naturally occurring Fc region that inherently exhibits decreased (or substantially no) binding to FcγRIIIA (CD16a) and/or reduced effector function (relative to the binding and effector function exhibited by the wild-type IgG1 Fc region (SEQ ID NO:72). In some aspects, the Fc constant region of the anti-FRα antibody or antigen-binding fragment in an anti-FRα active agent (e.g., an anti-FRα immunoconjugate) comprises an IgG2 Fc region (SEQ ID NO:73) or an IgG4 Fc region (SEQ ID NO:74). Since the N297A, N297G, N297Q, L234A, L235A and D265A substitutions abolish effector function, in circumstances in which effector function is desired, these substitutions may not be employed.

An IgG1 sequence for the CH2 and CH3 Domains of the Fc region-containing anti-FRα antibody or antigen-binding fragment in an anti-FRα active agent (e.g., an anti-FRα immunoconjugate) that has reduced or abolished effector function comprises the substitutions L234A/L235A (shown underlined in SEQ ID NO:75).

An IgG1 sequence for the CH2 and CH3 Domains of the Fc region-containing anti-FRα antibody or antigen-binding fragment in an anti-FRα active agent (e.g., an anti-FRα immunoconjugate) that has reduced or abolished effector function comprises the substitution N297A (shown underlined in SEQ ID NO:76).

An IgG1 sequence for the CH2 and CH3 Domains of the Fc region-containing anti-FRα antibody or antigen-binding fragment in an anti-FRα active agent (e.g., an anti-FRα immunoconjugate) that has reduced or abolished effector function comprises the substitution N297Q (shown underlined in SEQ ID NO:77).

In some aspects, the anti-FRα antibody or antigen-binding fragment in an anti-FRα active agent (e.g., an anti-FRα immunoconjugate) comprises an Fc (immunoglobulin) sequence selected from SEQ ID NO:75, SEQ ID NO:76, or SEQ ID NO:77.

In some aspects, a biparatopic anti-FRα antibody or antigen-binding fragment in a biparatopic anti-FRα active agent (e.g., a biparatopic anti-FRα immunoconjugate) comprises an Fc (immunoglobulin) sequence with reduced or abolished effector function (e.g., comprising the substitutions shown above in SEQ ID NO:75, SEQ ID NO:76, and/or SEQ ID NO:77) and comprises one or more knob-in-hole mutations as disclosed herein. In some aspects, the Fc sequence comprises a knob mutation as disclosed herein. In some aspects, the Fc sequence comprises a hole mutation as disclosed herein.

In some aspects, the anti-FRα antibody or antigen-binding fragment in an anti-FRα active agent (e.g., an anti-FRα immunoconjugate) comprises one or more modifications corresponding to: IgG1-C220S, C226S, C229S, P238S; IgG1-C226S, C229S; IgG1-C226S, C229S, E233P, L234V, L235A; IgG1-L234A, L235A; IgG1-L234F, L235E, P331S; IgG1-L234F, L235E, P331S; IgG1-H268Q, A330S, P331S; IgG1-G236R, L328R; IgG1-L235G, G236R, IgG1-N297A; IgG1-N325A, L328R; IgG1-N325L, L328R; IgG1-K326W, E333S; IgG2-V234A, G237A; IgG2-E333S; IgG2 H268Q, V309L, A330S, A331S; IgG4-S228P, L236E; IgG4-F234A, L235A; IgG4-F234A, G237A, E318A; IgG4-L235A, G237A, E318A; IgG4-L236E; IgG2-EU sequence 118-260; and IgG4-EU sequence 261-447; wherein the position numbering is according to the EU index as in Kabat.

In some aspects, the anti-FRα antibody or antigen-binding fragment in an anti-FRα active agent (e.g., an anti-FRα immunoconjugate) comprises a heavy chain immunoglobulin constant domain that has reduced CDC activity. In particular aspects, the anti-FRα antibody or antigen-binding fragment in an anti-FRα active agent (e.g., an anti-FRα immunoconjugate) comprises an IgG1 heavy chain constant region containing a mutation that decreases CDC activity (see, e.g., WO 1997/11971 and WO 2007/106585; U.S. Appl. Publ. 2007/0148167A1; McEarchern et al., Blood 109:1185-1192 (2007); Hayden-Ledbetter et al., Clin. Cancer 15:2739-2746 (2009); Lazar et al., PNAS USA 103:4005-4010 (2006); Bruckheimer et al., Neoplasia 11:509-517 (2009); Strohl, Curr. Op. Biotechnol. 20:685-691 (2009); and Sazinsky et al., PNAS USA 105:20167-20172 (2008); each of which is herein incorporated by reference in its entirety). Examples of heavy chain constant domain sequence modifications that decrease CDC include one or more modifications corresponding to: IgG1-C226S, C229S, E233P, L234V, L235A; IgG1-C226S, P230S; IgG1-L234F, L235E, P331S; IgG1-S239D, A330L, 1332E; IgG2 EU sequence 118-260; IgG4-EU sequence 261-447; and IgG2-H268Q, V309L, A330S, A331S, according to the EU index

In some aspects, the anti-FRα antibody or antigen-binding fragment in an anti-FRα active agent (e.g., an anti-FRα immunoconjugate) comprises a heavy chain immunoglobulin constant domain that contains one or more half-life extending amino acid modifications (e.g., substitutions). Numerous mutations capable of increasing the half-life of an Fc region-containing molecule are known in the art and are encompassed as components of the anti-FRα antibody or antigen-binding fragment in an anti-FRα active agent (e.g., an anti-FRα immunoconjugate) provided herein. See, e.g., U.S. Pat. Nos. 6,277,375; 7,083,784; 7,217,797, and 8,088,376; U.S. Publ. Nos. 2002/0147311; and 2007/0148164; and PCT Publication Nos. WO 1998/23289; WO 2009/058492; and WO 2010/033279, the contents of each of which is herein incorporated by reference in its entirety.

The serum half-life of proteins comprising Fc regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's (e.g., a human patient or other mammal) body or a specific compartment thereof, for example, as measured in serum, i.e., circulating half-life, or in other tissues. In general, an increase in half-life results in an increase in mean residence time (MRT) in circulation for the administered molecule.

In some aspects, the anti-FRα antibody or antigen-binding fragment in an anti-FRα active agent (e.g., an anti-FRα immunoconjugate) comprises a half-life extending amino acid substitution at one or more positions selected from the group consisting of: 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436, wherein the amino acid position numbering is according to the EU index. In some aspects, the anti-FRα antibody or antigen-binding fragment in an anti-FRα active agent (e.g., an anti-FRα immunoconjugate) contains one or more amino acid substitutions of amino acid residues at positions 251-257, 285-290, 308-314, 385-389, and 428-436, wherein the amino acid position numbering is according to the EU index. In some aspects, the anti-FRα antibody or antigen-binding fragment in an anti-FRα active agent (e.g., an anti-FRα immunoconjugate) contains one or more of a substitution of the amino acid at Kabat position 252 with Tyr, Phe, Trp, or Thr; a substitution of the amino acid at Kabat position 254 with Thr; a substitution of the amino acid at Kabat position 256 with Ser, Arg, Gln, Glu, Asp, or Thr; a substitution of the amino acid at Kabat position 257 with Leu; a substitution of the amino acid at Kabat position 309 with Pro; a substitution of the amino acid at Kabat position 311 with Ser; a substitution of the amino acid at Kabat position 428 with Thr, Leu, Phe, or Ser; a substitution of the amino acid at Kabat position 433 with Arg, Ser, Iso, Pro, or Gln; or a substitution of the amino acid at Kabat position 434 with Trp, Met, Ser, His, Phe, or Tyr. More specifically, the anti-FRα antibody or antigen-binding fragment in an anti-FRα active agent (e.g., an anti-FRα immunoconjugate) can contain amino acid substitutions relative to a wild-type human IgG constant domain including a substitution of the amino acid at Kabat position 252 with Tyr, a substitution of the amino acid at Kabat position 254 with Thr, and a substitution of the amino acid at Kabat position 256 with Glu.

In some aspects, the anti-FRα antibody or antigen-binding fragment in an anti-FRα active agent (e.g., an anti-FRα immunoconjugate) comprises a least one substitution selected from: T250Q, M252Y, S254T, T256E, K288D, T307Q, V308P, A378V, M428L, N434A, N434S, N434H, N434Y, H435K, and Y436I, wherein the numbering is that of the EU index as in Kabat. In some aspects, the anti-FRα antibody or antigen-binding fragment in an anti-FRα active agent (e.g., an anti-FRα immunoconjugate) comprises substitutions selected from: (a) M252Y, S254T and T256E; (b) M252Y and S254T; (c) M252Y and T256E; (d) T250Q and M428L; (e) T307Q and N434A; (f) A378V and N434A; (g) N434A and Y436I; (h) V308P and N434A; and (i) K288D and H435K.

In some aspects, the anti-FRα antibody or antigen-binding fragment in an anti-FRα active agent (e.g., an anti-FRα immunoconjugate) contains a variant IgG Fc Region comprising any 1, 2, or 3 of the substitutions: M252Y, S254T and T256E. The disclosure further provides an anti-FRα antibody or antigen-binding fragment in an anti-FRα active agent (e.g., an anti-FRα immunoconjugate) possessing variant Fc regions comprising: (a) one or more mutations which alter effector function and/or FcγR; and (b) one or more mutations which extend serum half-life.

TABLE 11
Immunoglobulin Sequences

| | |
|---|---|
| Exemplary IgG1 | APELLGGPSVFLFPPKPKDTLMISRTPEVTCVVVDVSHEDPEVKFN |
| Fc Region | WYVDGVEVHNAKTKPREEQYNSTYRVVSVLTVLHQDWLNGKEY |
| | KCKVSNKALPAPIEKTISKAKGQPREPQVYTLPPSRDELTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG (SEQ ID NO: 72) |
| Exemplary IgG2 | APPVAGPSVFLFPPKPKDTLMISRTPEVTCVVVDVSHEDPEVQFNW |
| Fc Region | YVDGVEVHNAKTKPREEQFNSTFRVVSVLTVVHQDWLNGKEYKC |
| | KVSNKGLPAPIEKTISKTKGQPREPQVYTLPPSREEMTKNQVSLTCL |
| | VKGFYPSDIAVEWESNGQPENNYKTTPPMLDSDGSFFLYSKLTVDK |
| | SRWQQGNVFSCSVMHEALHNHYTQKSLSLSPG (SEQ ID NO: 73) |
| Exemplary IgG4 | APEFLGGPSVFLFPPKPKDTLMISRTPEVTCVVVDVSQEDPEVQFNW |
| Fc Region | YVDGVEVHNAKTKPREEQFNSTYRVVSVLTVLHQDWLNGKEYKC |
| | KVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDK |
| | SRWQEGNVFSCSVMHEALHNHYTQKSLSLSLG (SEQ ID NO: 74) |
| Exemplary | APEAAGGPSVFLFPPKPKDTLMISRTPEVTCVVVDVSHEDPEVKFN |
| L234A/L235A | WYVDGVEVHNAKTKPREEQYNSTYRVVSVLTVLHQDWLNGKEY |
| IgG1 Fc Region | KCKVSNKALPAPIEKTISKAKGQPREPQVYTLPPSRDELTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG (SEQ ID NO: 75) |
| Exemplary | APELLGGPSVFLFPPKPKDTLMISRTPEVTCVVVDVSHEDPEVKFN |
| N297A IgG1 Fc | WYVDGVEVHNAKTKPREEQYASTYRVVSVLTVLHQDWLNGKEY |
| Region | KCKVSNKALPAPIEKTISKAKGQPREPQVYTLPPSRDELTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG (SEQ ID NO: 76) |
| Exemplary | APELLGGPSVFLFPPKPKDTLMISRTPEVTCVVVDVSHEDPEVKFN |
| N297Q IgG1 Fc | WYVDGVEVHNAKTKPREEQYQSTYRVVSVLTVLHQDWLNGKEY |
| Region | KCKVSNKALPAPIEKTISKAKGQPREPQVYTLPPSRDELTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG (SEQ ID NO: 77) |

In some aspects, an anti-FRα active agent is comprises an anti-FRα antibody or antigen-binding fragment and a cytotoxic agent. The cytotoxic agent may be coupled or conjugated either directly to the anti-FRα-antibody or antigen-binding fragment or indirectly, through a linker using techniques known in the art to produce an “immunoconjugate,” “conjugate,” or “ADC.” Suitable cytotoxic agents can be any compound that results in the death of a cell, or induces cell death, or in some manner decreases cell viability, and includes, for example, maytansinoids and maytansinoid analogs.

### A. Exemplary Immunoconjugates

As provided herein, an anti-FRα immunoconjugate can comprise an anti-FRα antibody or antigen-binding fragment thereof linked to a maytansinoid.

The anti-FRα immunoconjugate can comprise a maytansinoid linked to an anti-FRα antibody or antigen-binding fragment thereof comprising (a) VH CDR1, VH CDR2, and VH CDR3 comprising the amino acid sequences of SEQ ID NOs:10-12, respectively; and (b) VL CDR1, VL CDR2, and VL CDR3 comprising the amino acid sequences of SEQ ID NOs:15-17, respectively, wherein the maytansinoid is DM4 and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited CDR sequences and a DM4 maytansinoid and/or a sulfo-SPDB linker can be administered at a dose of 6 mg/kg adjusted ideal body weight (AIBW) or 5 mg/kg AIBW as disclosed, e.g., in WO 2015/054400. In some aspects, such an immunoconjugate comprising the recited CDR sequences and a DM4 maytansinoid and/or a sulfo-SPDB linker is administered at a dose of 6 mg/kg AIBW every three weeks. In some aspects, such an immunoconjugate comprising the recited CDR sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) VH CDR1, VH CDR2, and VH CDR3 comprising the amino acid sequences of SEQ ID NOs:13, 14, and 12, respectively; and (b) VL CDR1, VL CDR2, and VL CDR3 comprising the amino acid sequences of SEQ ID NOs:15-17, respectively, wherein the maytansinoid is DM4, and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited CDR sequences and a DM4 maytansinoid and/or a sulfo-SPDB linker can be administered at a dose of 6 mg/kg AIBW. In some aspects, such an immunoconjugate comprising the recited CDR sequences and a DM4 maytansinoid and/or a sulfo-SPDB linker is administered at a dose of 6 mg/kg AIBW every three weeks. In some aspects, such an immunoconjugate comprising the recited CDR sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) VH CDR1, VH CDR2, and VH CDR3 comprising the amino acid sequences of SEQ ID NOs:10, 14, and 12, respectively; and (b) VL CDR1, VL CDR2, and VL CDR3 comprising the amino acid sequences of SEQ ID NOs:15-17, respectively, wherein the maytansinoid is DM4 and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited CDR sequences and a DM4 maytansinoid and/or a sulfo-SPDB linker can be administered at a dose of 6 mg/kg AIBW. In some aspects, such an immunoconjugate comprising the recited CDR sequences and a DM4 maytansinoid and/or a sulfo-SPDB linker is administered at a dose of 6 mg/kg AIBW every three weeks. In some aspects, such an immunoconjugate comprising the recited CDR sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) VH CDR1, VH CDR2, and VH CDR3 comprising the amino acid sequences of SEQ ID NOs:13, 11, and 12, respectively; and (b) VL CDR1, VL CDR2, and VL CDR3 comprising the amino acid sequences of SEQ ID NOs:15-17, respectively, wherein the maytansinoid is DM4 and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited CDR sequences and a DM4 maytansinoid and/or a sulfo-SPDB linker can be administered at a dose of 6 mg/kg AIBW. In some aspects, such an immunoconjugate comprising the recited CDR sequences and a DM4 maytansinoid and/or a sulfo-SPDB linker is administered at a dose of 6 mg/kg AIBW every three weeks. In some aspects, such an immunoconjugate comprising the recited CDR sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) a VH comprising the amino acid sequences of SEQ ID NO:38; and (b) a VL comprising the amino acid sequences of SEQ ID NO:44, wherein the maytansinoid is DM4 and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited VH and VL sequences and a DM4 maytansinoid and/or a sulfo-SPDB linker can be administered at a dose of 6 mg/kg AIBW. In some aspects, such an immunoconjugate comprising the recited VH and VL sequences and a DM4 maytansinoid and/or a sulfo-SPDB linker is administered at a dose of 6 mg/kg AIBW every three weeks. In some aspects, such an immunoconjugate comprising the recited VH and VL sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) a heavy chain (HC) comprising the amino acid sequences of SEQ ID NO:50; and (b) a light chain (LC) comprising the amino acid sequences of SEQ ID NO:56, wherein the maytansinoid is DM4, and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited HC and LC sequences and a DM4 maytansinoid and/or a sulfo-SPDB linker can be administered at a dose of 6 mg/kg AIBW. In some aspects, such an immunoconjugate comprising the recited HC and LC sequences and a DM4 maytansinoid and/or a sulfo-SPDB linker is administered at a dose of 6 mg/kg AIBW every three weeks. In some aspects, such an immunoconjugate comprising the recited HC and LC sequences and a DM4 maytansinoid and/or a sulfo-SPDB linker is administered at a dose of 6 mg/kg AIBW every four weeks.

The anti-FRα immunoconjugate can comprise a maytansinoid linked to an anti-FRα antibody or antigen-binding fragment thereof comprising (a) a heavy chain (HC) comprising the same amino acid sequence as the amino acid sequence of the heavy chain encoded by the plasmid deposited with the American Type Culture Collection (ATCC) as PTA-10772 and (b) a light chain (LC) comprising the same amino acid sequence as the amino acid sequence of the light chain encoded by the plasmid deposited with the ATCC as PTA-10774, wherein the maytansinoid is DM4 and/or the maytansinoid is linked to the antibody or antigen-binding fragment thereof via a sulfo-SPDB linker. The immunoconjugate can comprise 1-10, 2-5, or 3-4 maytansinoids. Such an immunoconjugate comprising the recited HC and LC sequences and a DM4 maytansinoid and/or a sulfo-SPDB linker can be administered at a dose of 6 mg/kg AIBW. In some aspects, such an immunoconjugate comprising the recited HC and LC sequences and a DM4 maytansinoid and/or a sulfo-SPDB linker is administered at a dose of 6 mg/kg AIBW every three weeks. In some aspects, such an immunoconjugate comprising the recited HC and LC sequences and a DM4 maytansinoid and/or a sulfo-SPDB linker is administered at a dose of 6 mg/kg AIBW every four weeks.

The anti-FRα active agent (e.g., anti-FRα immunoconjugate) can be IMGN853. IMGN853 can be administered at a dose of 6 mg/kg AIBW. In some aspects, IMGN853 is administered at a dose of 6 mg/kg AIBW every three weeks. In some aspects, IMGN853 is administered at a dose of 6 mg/kg AIBW every four weeks.

The anti-FRα immunoconjugate can comprise a maytansinoid linked to a biparatopic anti-FRα antibody or antigen-binding fragment thereof can comprising (a) VH CDR1, VH CDR2, and VH CDR3 comprising the amino acid sequences of SEQ ID NOs:2-4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10-12, respectively; and (d) VL CDR1, VL CDR2, and VL CDR3 comprising the amino acid sequences of SEQ ID NOs:15-17, respectively. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof can comprising (a) VH CDR1, VH CDR2, and VH CDR3 comprising the amino acid sequences of SEQ ID NOs:5, 6, and 4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:13, 14, and 12, respectively; and (d) VL CDR1, VL CDR2, and VL CDR3 comprising the amino acid sequences of SEQ ID NOs:15-17, respectively. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof can comprising (a) VH CDR1, VH CDR2, and VH CDR3 comprising the amino acid sequences of SEQ ID NOs:2, 6, and 4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10, 14, and 12, respectively; and (d) VL CDR1, VL CDR2, and VL CDR3 comprising the amino acid sequences of SEQ ID NOs:15-17, respectively. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof can comprising (a) a VH comprising the amino acid sequence of SEQ ID NO:37; (b) a VL comprising the amino acid sequence of SEQ ID NO:43; (c) a VH comprising the amino acid sequence of SEQ ID NO:38; and (d) a VL comprising the amino acid sequence of SEQ ID NO:44. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof can comprising (a) an scFv comprising the amino acid sequence of SEQ ID NO:60; (b) a VH comprising the amino acid sequence of SEQ ID NO:38; and (c) a VL comprising the amino acid sequence of SEQ ID NO:44. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof comprising (a) a polypeptide comprising the amino acid sequence of SEQ ID NO:61; (b) a polypeptide comprising the amino acid sequence of SEQ ID NO:62, and (c) a polypeptide comprising the amino acid sequence of SEQ ID NO:56. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα immunoconjugate can comprise a maytansinoid linked to a biparatopic anti-FRα antibody or antigen-binding fragment thereof comprising (a) a polypeptide comprising the same amino acid sequence as the amino acid sequence encoded by the plasmid deposited with the American Type Culture Collection (ATCC) as PTA-125915; (b) a polypeptide comprising the same amino acid sequence as the amino acid sequence encoded by the plasmid deposited with the ATCC as PTA-125915, and (c) a polypeptide comprising the amino acid sequence as the amino acid sequence of the light chain encoded by the plasmid deposited with the ATCC as PTA-10774. The maytansinoid can be, for example, DM21. The maytansinoid can be linked to the antibody or antigen-binding fragment thereof via a GMBS or sulfo-GMBS linker. The immunoconjugate can comprise 1-10, 2-5, or 3-4 maytansinoids.

The anti-FRα active agent (e.g., anti-FRα immunoconjugate) can be IMGN151.

In a first particular aspect (A1), an immunoconjugate provided herein comprises an anti-FRα antibody or antigen binding fragment thereof described herein covalently linked to a maytansinoid compound through the ε-amino group of one or more lysine residues located on the anti-FRα antibody or antigen binding fragment thereof. In some aspects, the immunoconjugate is represented by formula (I):

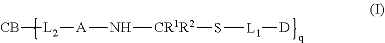

or a pharmaceutically acceptable salt thereof, wherein:
   CB is a an anti-FRα antibody or antigen binding fragment thereof (e.g., a biparatopic anti-FRα antibody or antigen binding fragment thereof);
   L₂ is represented by one of the following formula:

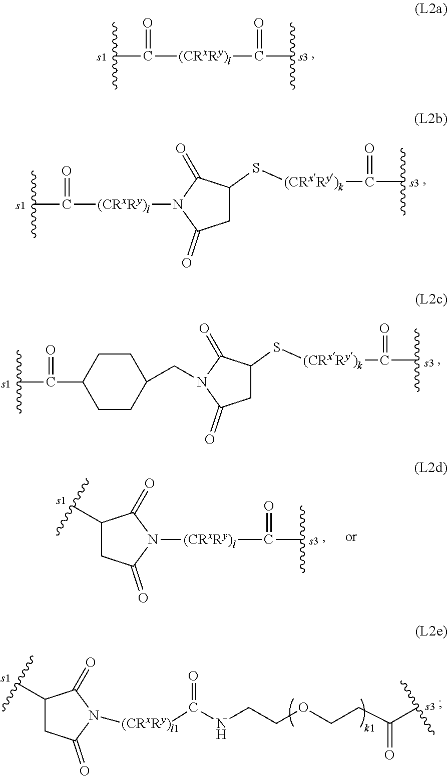

wherein:
R^{x}, R^{y}, R^{x′} and R^{y′}, for each occurrence, are independently H, —OH, halogen, —O—(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;
l1 is an integer from 2 to 5;
k1 is an integer from 1 to 5; and
s1 indicates the site connected to the cell-binding agent CB and s3 indicates the site connected to the A group;
A is an amino acid residue or a peptide comprising 2 to 20 amino acid residues;
R¹ and R² are each independently H or a C₁₋₃alkyl;
L₁ is represented by the following formula:

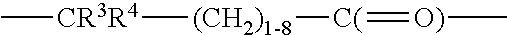

wherein R³ and R⁴ are each independently H or Me, and the —C(═O)— moiety in L₁ is connected to D;
D is represented by the following formula:

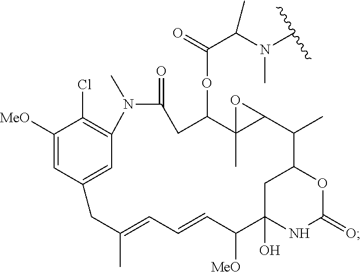 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

q is an integer from 1 to 20. In some aspects q is an integer from 1 to 10. In some aspects q is an integer from 2 to 5. In some aspects, q is an integer from 3 to 4.

In a 1^{st} specific aspect of the first particular aspect (A1-1), an immunoconjugate provided herein is represented by formula (I) described above, wherein R^{x}, R^{y}, R^{x′} and R^{y′} are all H; and l and k are each independently an integer an integer from 2 to 6; and the remaining variables are as described above for formula (I).

In a 2^{nd} specific aspect of the first particular aspect (A1-2), an immunoconjugate provided herein is represented by formula (I) described above, wherein A is a peptide containing 2 to 5 amino acid residues; and the remaining variables are as described above for formula (I) in the first particular aspect (A1) or the i^{st} specific aspect of the first particular aspect (A1-1). In some aspects, A is a peptide cleavable by a protease. In some aspects, a peptide cleavable by a protease expressed in tumor tissue. In some aspects, A is a peptide having an amino acid that is covalently linked with —NH—CR¹R²—S-L₁-D selected from the group consisting of Ala, Arg, Asn, Asp, Cit, Cys, selino-Cys, Gln, Glu, Gly, Ile, Leu, Lys, Met, Phe, Pro, Ser, Thr, Trp, Tyr and Val, each independently as L or D isomer. In some aspects, the amino acid connected to —NH—CR¹R²—S-L₁-D is an L amino acid.

In a 3^{rd} specific aspect of the first particular aspect (A1-3), an immunoconjugate provided herein is represented by formula (I) described above, wherein A is selected from the group consisting of Gly-Gly-Gly, Ala-Val, Val-Ala, D-Val-Ala, Val-Cit, D-Val-Cit, Val-Lys, Phe-Lys, Lys-Lys, Ala-Lys, Phe-Cit, Leu-Cit, Ile-Cit, Phe-Ala, Phe-N9-tosyl-Arg, Phe-N9-nitro-Arg, Phe-Phe-Lys, D-Phe-Phe-Lys, Gly-Phe-Lys, Leu-Ala-Leu, Ile-Ala-Leu, Val-Ala-Val, Ala-Ala-Ala, D-Ala-Ala-Ala, Ala-D-Ala-Ala, Ala-Ala-D-Ala, Ala-Leu-Ala-Leu (SEQ ID NO:78), 0-Ala-Leu-Ala-Leu (SEQ ID NO:79), Gly-Phe-Leu-Gly (SEQ ID NO:63), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Gln-Val, Asn-Ala, Gln-Phe, Gln-Ala, D-Ala-Pro, and D-Ala-tBu-Gly, wherein the first amino acid in each peptide is connected to L₂ group and the last amino acid in each peptide is connected to —NH-CR¹R₂—S-L₁-D; and the remaining variables are as described for formula (I) in the first particular aspect (A1) or the 1^{st} specific aspect of the first particular aspect (A1-1).

In a 4^{th} specific aspect of the first particular aspect (A1-4), an immunoconjugate provided herein is represented by formula (I) described above, wherein R¹ and R² are both H; and the remaining variables are as described for formula (I) in the first particular aspect (A1), the 1^{st}specific aspect of the first particular aspect (A1-1), the 2^{nd} specific aspect of the first particular aspect (A1-2), or the 3^{rd} specific aspect of the first particular aspect (A1-3).

In a 5^{th} specific aspect of the first particular aspect (A1-5), an immunoconjugate provided herein is represented by formula (I) described above, wherein L₁ is —(CH₂)₄₋₆—C(═O)—; and the remaining variables are as described for formula (I) in the first particular aspect (A1), the 1^{st} specific aspect of the first particular aspect (A1-1), the 2^{nd} specific aspect of the first particular aspect (A1-2), the 3^{rd} specific aspect of the first particular aspect (A1-3), or the₄′ specific aspect of the first particular aspect (A1-4).

In a 6^{th} specific aspect of the first particular aspect (A1-6), an immunoconjugate provided herein is represented by formula (I) described above, wherein D is represented by the following formula:

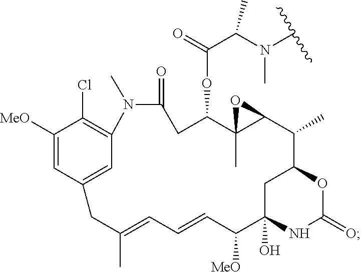 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

and the remaining variables are as described for formula (I) in the first particular aspect (A1), the 1^{st} specific aspect of the first particular aspect (A1-1), the 2^{nd} specific aspect of the first particular aspect (A1-2), the 3^{rd} specific aspect of the first particular aspect (A1-3), the 4^{th} specific aspect of the first particular aspect (A1-4) or the 5^{th} specific aspect of the first particular aspect (A1-5).

In a 7^{th} specific aspect (A7), an immunoconjugate provided herein is represented by the following formula:

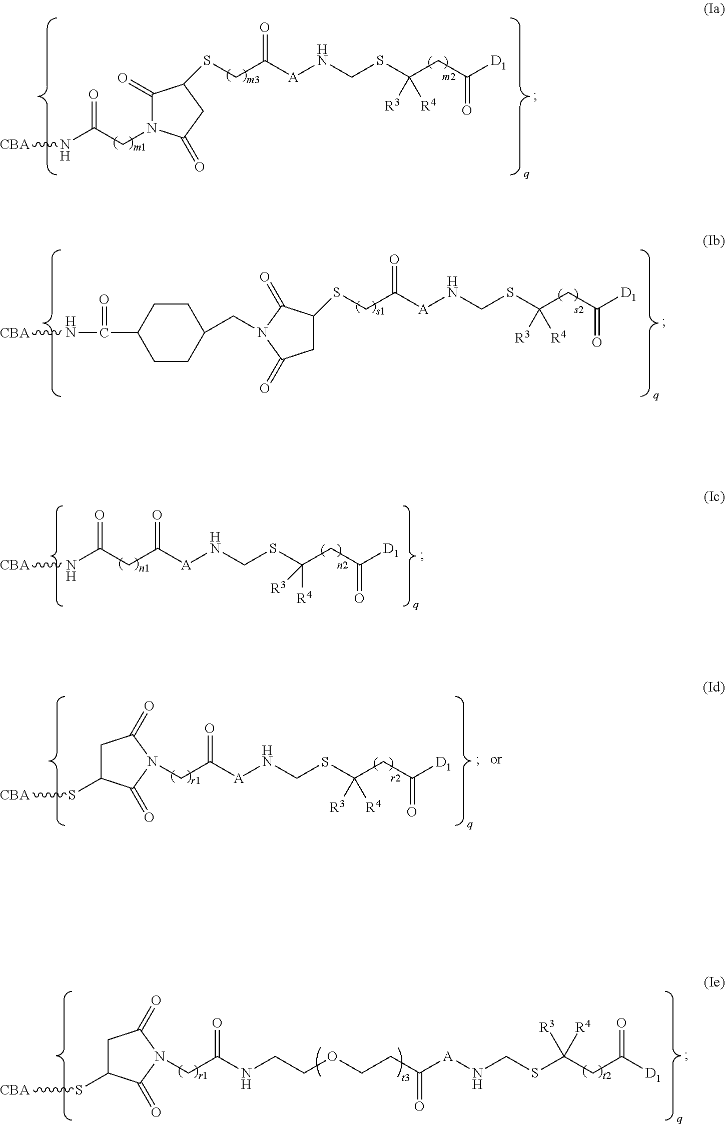

or a pharmaceutically acceptable salt thereof, wherein:

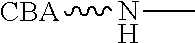

is the anti-FRα antibody or antigen-binding fragment thereof (e.g., biparatopic anti-FRα antibody or antigen binding fragment thereof) connected to the L₂ group through a Lys amine group;

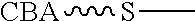

is the anti-FRα antibody or antigen-binding fragment thereof (e.g., biparatopic anti-FRα antibody or antigen binding fragment thereof) connected to the L₂ group through a Cys thiol group;
R³ and R⁴ are each independently H or Me;
m1, m3, n1, r1, s1 and t1 are each independently an integer from 1 to 6;
m2, n2, r2, s2 and t2 are each independently an integer from 1 to 7;
t3 is an integer from 1 to 12;
D1 is represented by the following formula:

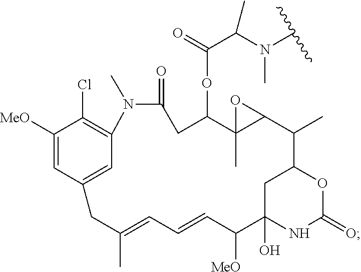 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

and
q is an integer from 1 to 20. In some aspects q is an integer from 1 to 10. In some aspects q is an integer from 2 to 5. In some aspects, q is an integer from 3 to 4. In some aspects, D₁ is represented by the following formula:

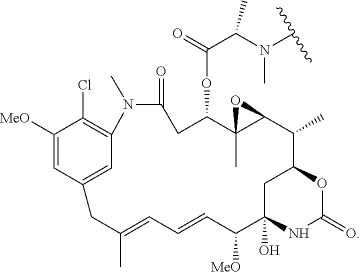 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In an 8^{th} specific aspect (A8), an immunoconjugate provided herein is represented by the following formula:

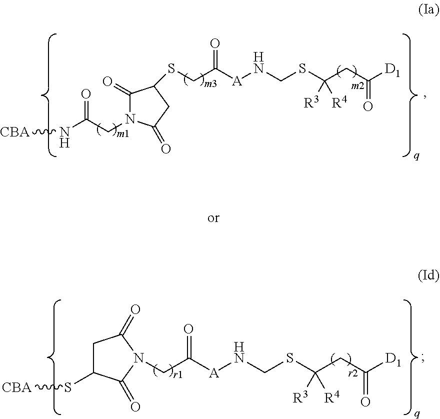

wherein:
   m1 and m3 are each independently an integer from 2 to 4;
   m2 is an integer from 2 to 5;
   r1 is an integer from 2 to 6;
   r2 is an integer from 2 to 5; and
      the remaining variables are as described in the 7^{th} specific aspect (A7).

In a₉^{th} specific aspect (A9), for the immunoconjugates described in the 7^{th} or 8^{th} specific aspects (A7 or A8), A is Ala-Ala-Ala, Ala-D-Ala-Ala, Ala-Ala, D-Ala-Ala, Val-Ala, D-Val-Ala, D-Ala-Pro, or D-Ala-tBu-Gly. In some aspects, for the immunoconjugates described in the 7^{th} or 8^{th} specific aspects (A7 or A8), A is L-Ala-D-Ala-L-Ala.

In a 10^{th} specific aspect (A10), an immunoconjugate provided herein is represented by the following formula:

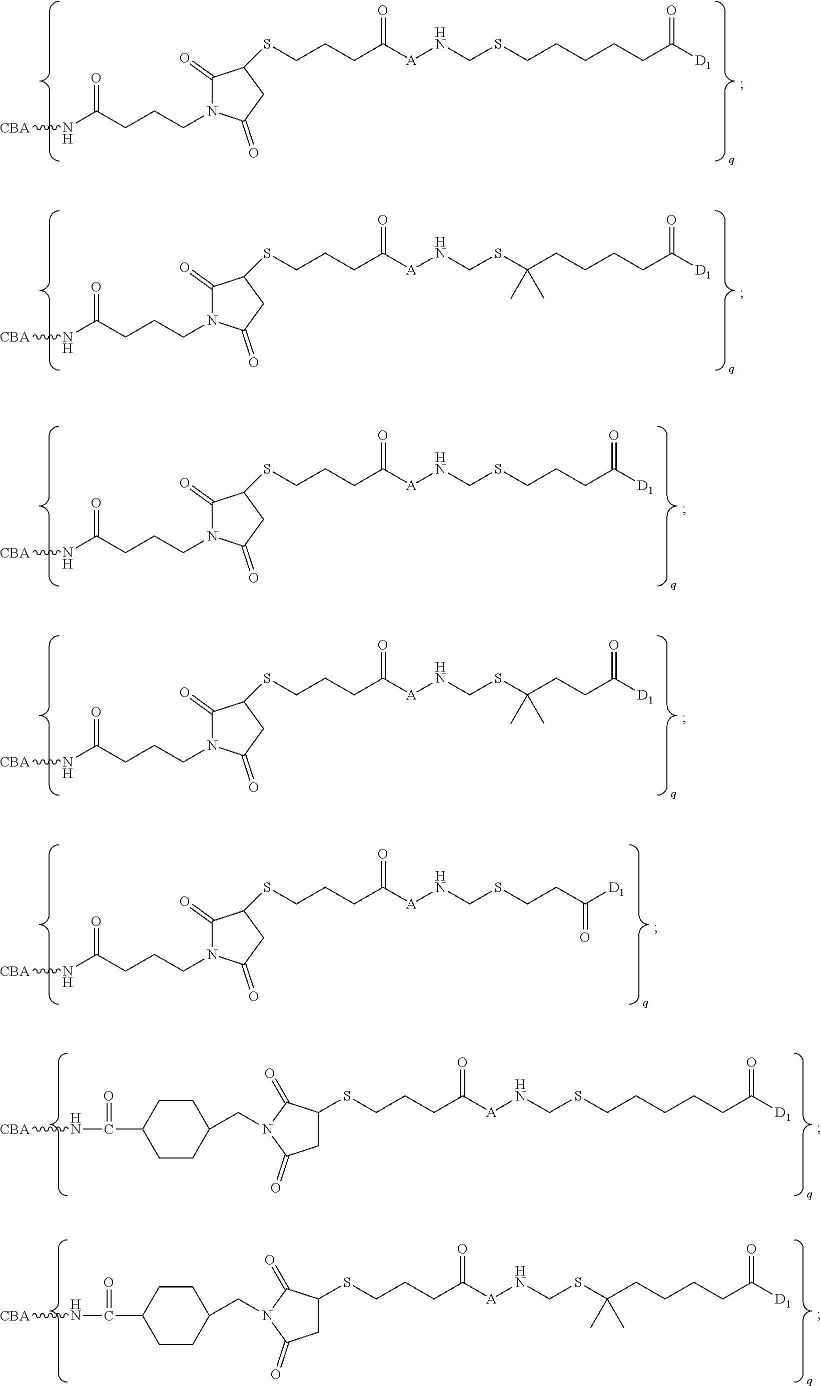

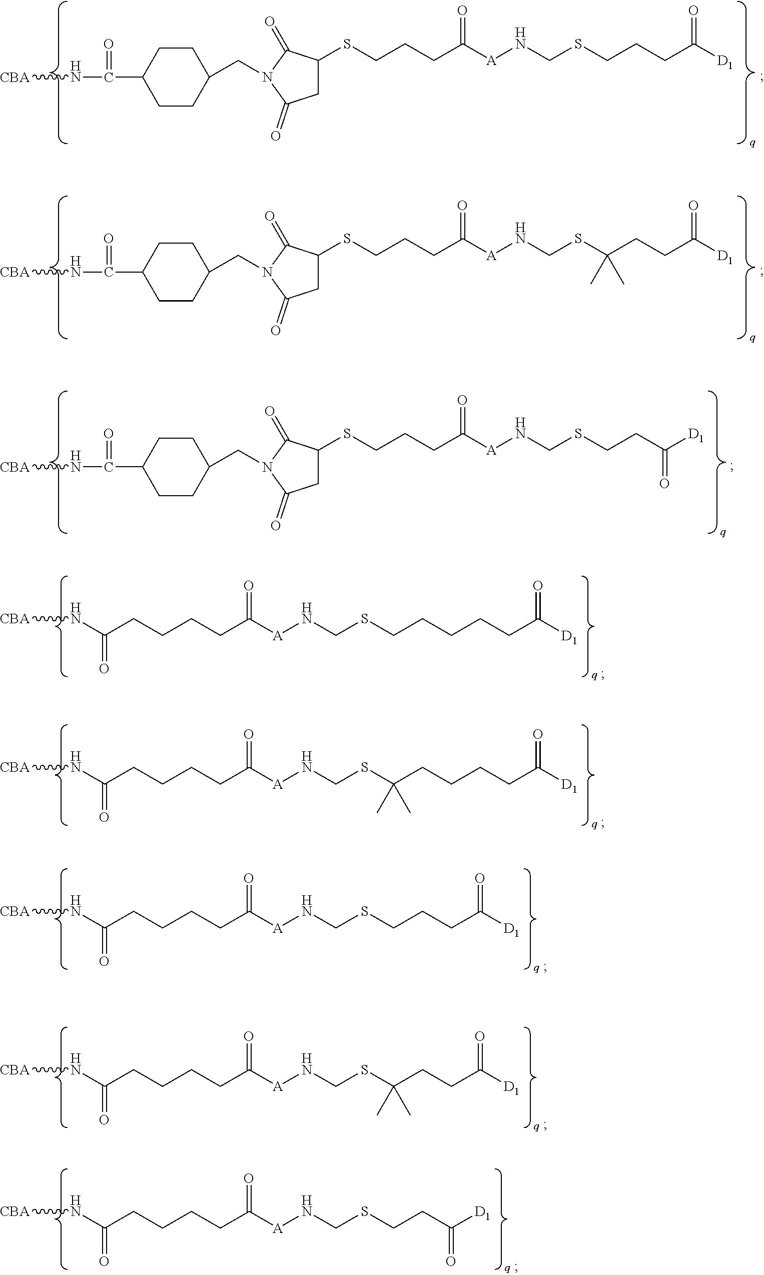

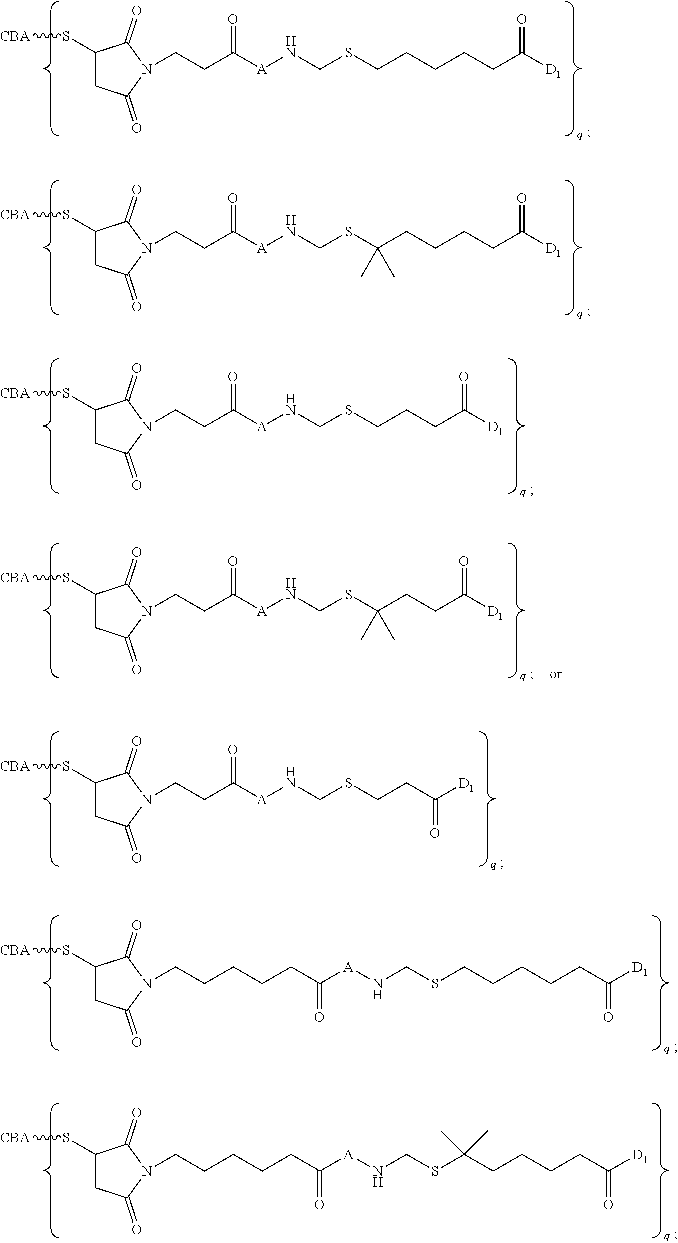

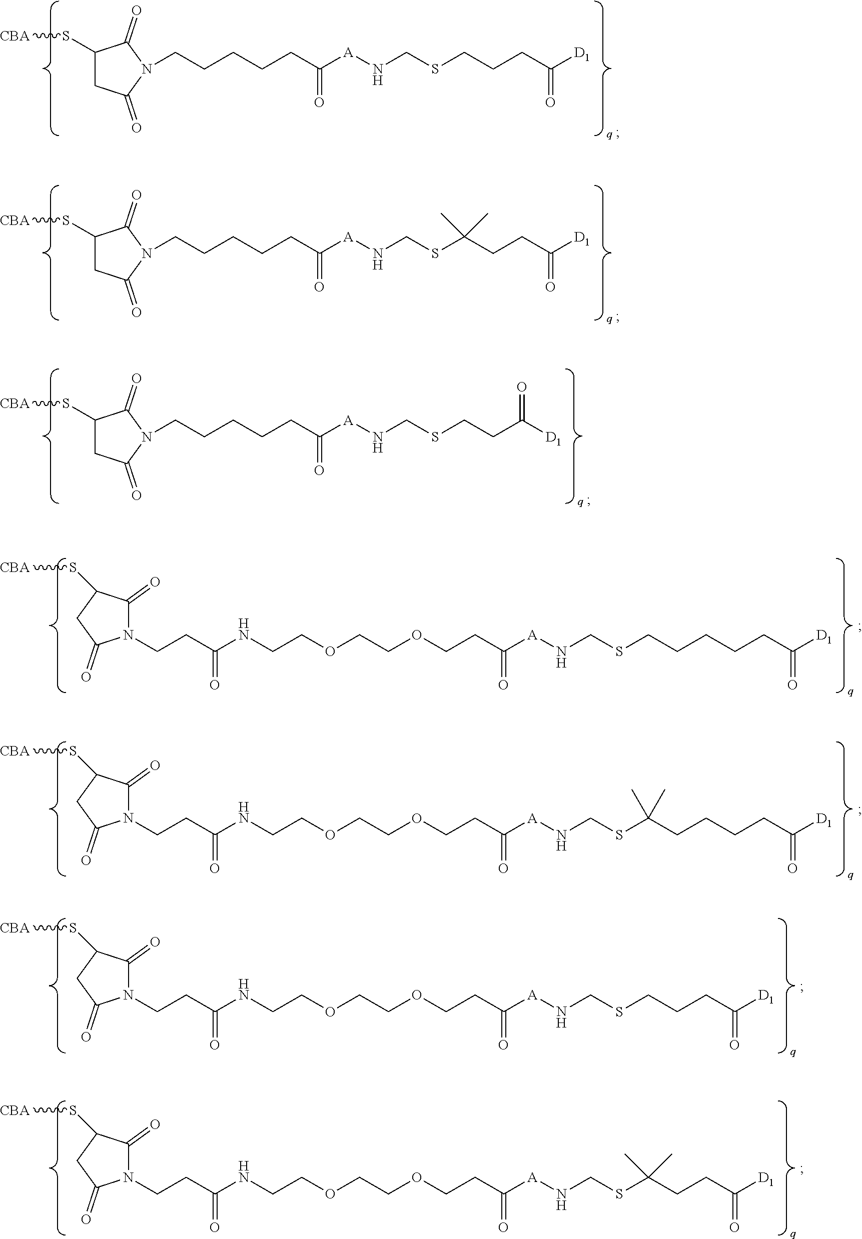

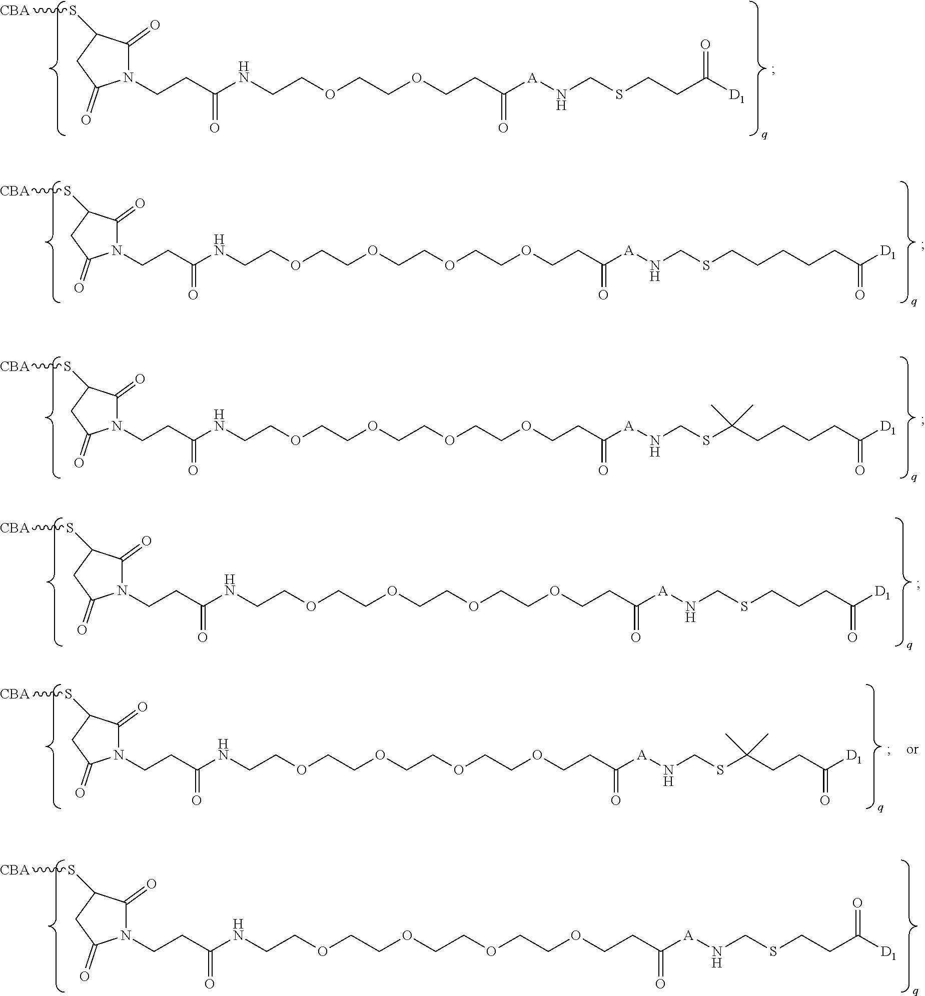

or a pharmaceutically acceptable salt thereof, wherein:
   A is Ala-Ala-Ala, Ala-D-Ala-Ala, Ala-Ala, D-Ala-Ala, Val-Ala, D-Val-Ala, D-Ala-Pro, or D-Ala-tBu-Gly, and
   D₁ is represented by the following formula:

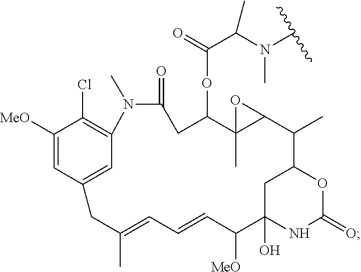 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

and the remaining variables are as described in the 7^{th}, 8^{th} or 9^{th} specific aspects (A7, A8, or A9). In some aspects, A is L-Ala-D-Ala-L-Ala. In some aspects, D₁ is represented by the following formula:

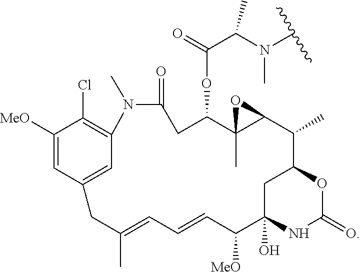 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In an 11^{th} specific aspect (A11), an immunoconjugate provided herein is represented by the following formula:

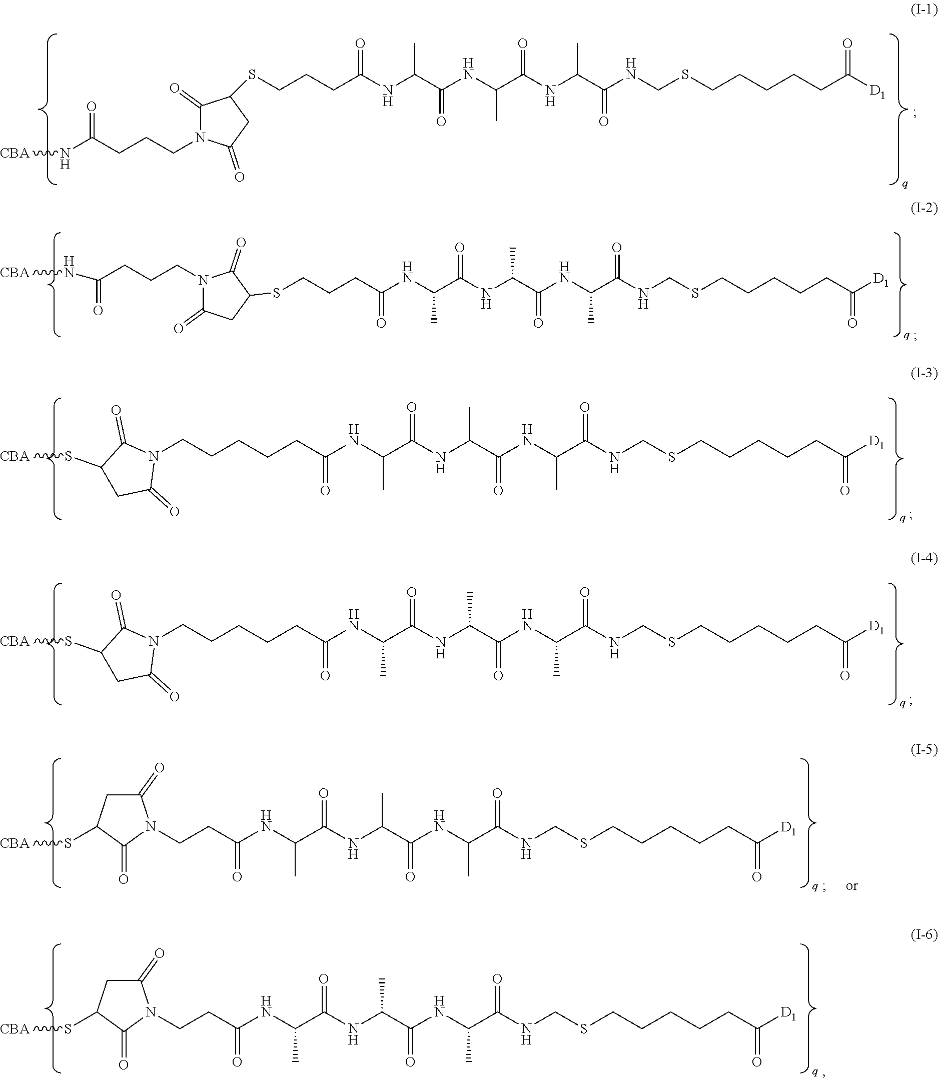

wherein D₁ is represented by the following formula:

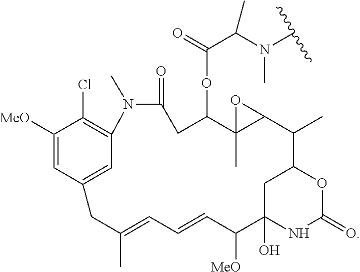 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

In some aspects, D₁ is represented by the following formula:

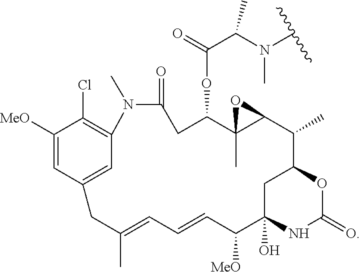 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In a 12^{th} specific aspect (A12), an immunoconjugate provided herein is represented by the following formula:

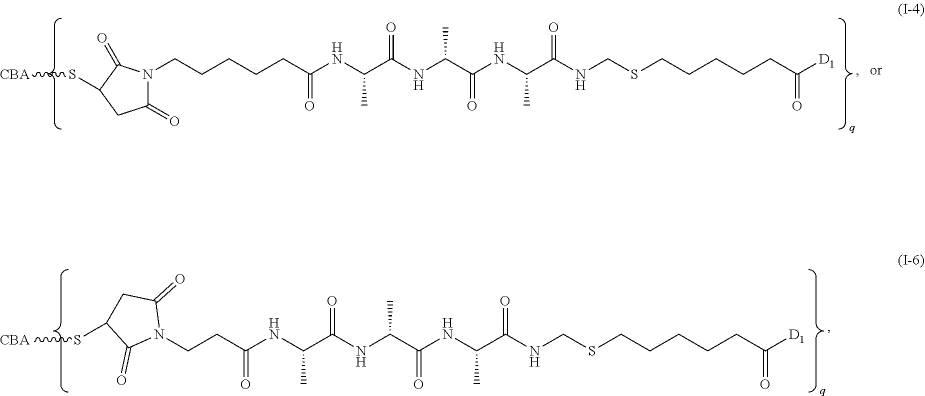

wherein:
CBA is a biparatopic anti-FRα antibody or antigen-binding fragment thereof, wherein said antibody or antigen-binding fragment thereof comprises (a) VH CDR1, VH CDR2, and VH CDR3 comprising the amino acid sequences of SEQ ID NOs:2-4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10-12, respectively; and (d) VL CDR1, VL CDR2, and VL CDR3 comprising the amino acid sequences of SEQ ID NOs:15-17, respectively;
q is 1 or 2;
D₁ is represented by the following formula:

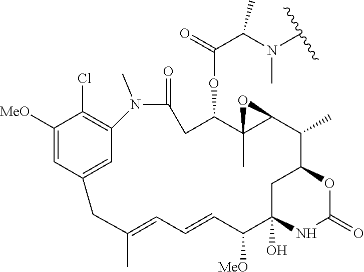 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some aspects, for the immunoconjugate of formula (I-4) or (I-6), the a biparatopic anti-FRα antibody or antigen-binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38.

In a 13^{th} specific aspect (A13), an immunoconjugate provided herein is represented by the following formula:

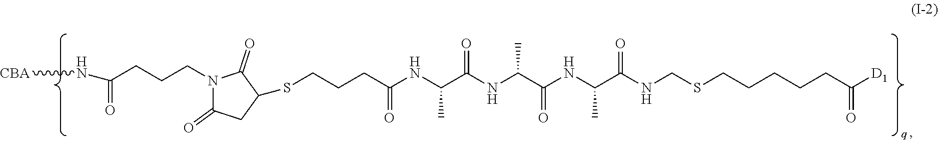

wherein:
CBA is a biparatopic anti-FRα antibody or antigen-binding fragment thereof, wherein said antibody or antigen-binding fragment thereof comprises (a) VH CDR1, VH CDR2, and VH CDR3 comprising the amino acid sequences of SEQ ID NOs:2-4, respectively; (b) VL CDR1, VL CDR2, and VL CDR3 comprising the amino acid sequences of SEQ ID NOs:7-9, respectively; (c) VH CDR1, VH CDR2, and VH CDR3 comprising the amino acid sequences of SEQ ID NOs:10-12, respectively; and (d) VL CDR1, VL CDR2, and VL CDR3 comprising the amino acid sequences of SEQ ID NOs:15-17, respectively;
q is an integer from 1 to 10, e.g., 1 or 10; and
D₁ is represented by the following formula:

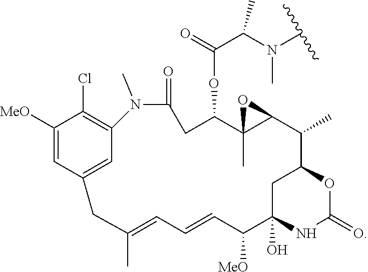 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some aspects, for the immunoconjugate of formula (I-2), the a biparatopic anti-FRα antibody or antigen-binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38. In some aspects, for the immunoconjugate of formula (I-2), the a biparatopic anti-FRα antibody or antigen-binding fragment thereof comprises polypeptides having the amino acid sequences of SEQ ID NOs:61, 62, and 56.

In a 14^{th} aspect (A14), an immunoconjugate provided herein comprises an biparatopic anti-FRα antibody coupled to a maytansinoid compound DM21C (also referred to as Mal-LDL-DM or MalC5-LDL-DM or compound 17a) represented by the following structural formula:

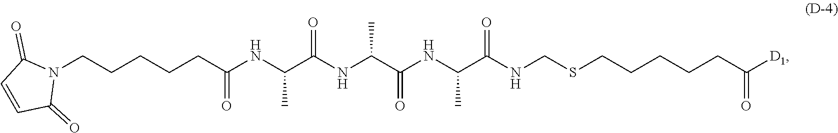

wherein the biparatopic anti-FRα antibody or antigen binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38; and D₁ is represented by the following formula:

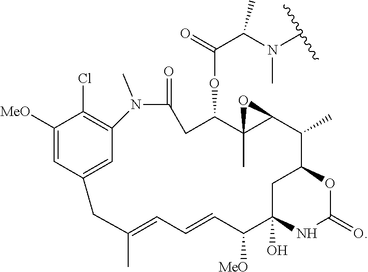 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some aspects, the immunoconjugate is represented by the following structural formula:

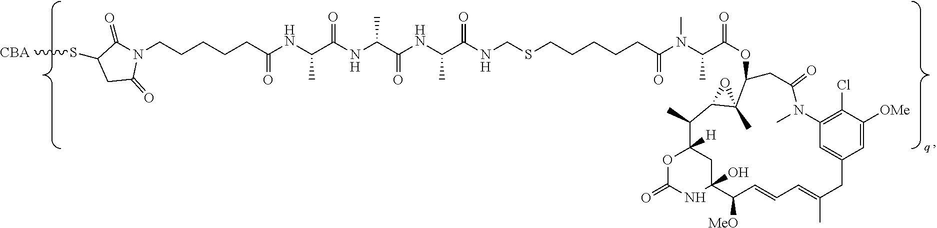

wherein:
CBA is a biparatopic anti-FRα antibody or antigen binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38; and
q is 1 or 2.

In some aspects, for compositions (e.g., pharmaceutical compositions) comprising immunoconjugates of the 14^{th} specific aspect (A14), DAR is in the range of 1.5 to 2.2, 1.7 to 2.2 or 1.9 to 2.1. In some aspects, the DAR is 1.7, 1.8, 1.9, 2.0 or 2.1.

In a 15^{th} specific aspect (A15), an immunoconjugate provided herein comprises a biparatopic anti-FRα antibody or antigen-binding fragment thereof coupled to a maytansinoid compound DM21 (also referred to as DM21L, LDL-DM, DM21L-G, or compound 14c) represented by the following structural formula:

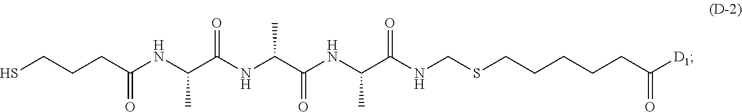

via γ-maleimidobutyric acid N-succinimidyl ester (GMBS) or a N-(γ-maleimidobutryloxy)sulfosuccinimide ester (sulfo-GMBS or sGMBS) linker. The biparatopic anti-FRα antibody or antigen binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38.

The GMBS and sulfo-GMBS (or sGMBS) linkers are known in the art and can be presented by the following structural formula:

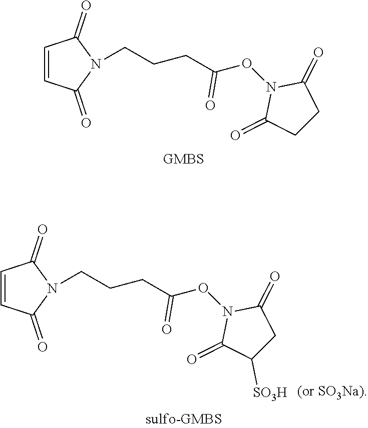 [SMILES: O=C(CCCN1C(=O)C=CC1=O)ON1C(=O)CCC1=O ; O=C(CCCN1C(=O)C=CC1=O)ON1C(=O)CC(S(=O)(=O)O)C1=O]

In some aspects, the immunoconjugate is represented by the following structural formula:

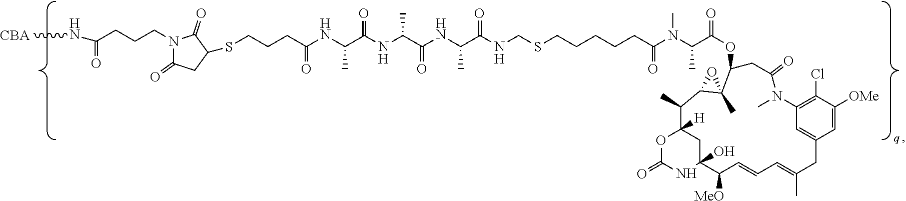

wherein:
CBA is a biparatopic anti-FRα antibody or antigen binding fragment thereof comprises a VL comprising the amino acid sequence of SEQ ID NO:43, a VH comprising the amino acid sequence of SEQ ID NO:37, a VL comprising the amino acid sequence of SEQ ID NO:44, and a VH comprising the amino acid sequence of SEQ ID NO:38; and
q is an integer from 1 to 10, e.g., 1 or 10. In some aspects q is an integer from 2 to 5. In some aspects, q is an integer from 3 to 4.

In some aspects, for immunoconjugates of the 15^{th} specific aspect (A15), the a biparatopic anti-FRα antibody or antigen-binding fragment thereof comprises polypeptides having the amino acid sequences of SEQ ID NOs: 61, 62, and 56.

In some aspects, for compositions (e.g., pharmaceutical compositions) comprising immunoconjugates of the 15^{th} specific aspect (A15), DAR is in the range of 3.0 to 4.0, 3.2 to 3.8, 3.1 to 3.7, or 3.4 to 3.7. In some aspects, the DAR is 3.2, 3.3, 3.4, 3.5, 3.5, 3.7, or 3.8. In some aspects, the DAR is 3.5.

In some aspects, for compositions comprising lysine conjugates, DAR is in the range of 1.5 to 3.1. In some aspects, the DAR is about 2.0.

The anti-FRα immunoconjugate can comprise eribulin mesylate linked to an anti-FRα antibody or antigen-binding fragment thereof comprising (a) VH CDR1, VH CDR2, and VH CDR3 comprising the amino acid sequences of SEQ ID NOs:80 or 83, 81 or 84, and 82 or 85, respectively; and (b) VL CDR1, VL CDR2, and VL CDR3 comprising the amino acid sequences of SEQ ID NOs:86 or 89, 87 or 90, and 88, respectively. The eribulin mesylate can be linked to the antibody or antigen-binding fragment thereof via a cathepsin-B linker. The immunoconjugate can comprise 1-10, 2-5, or 4 eribulin mesylate.

The anti-FRα immunoconjugate can comprise eribulin mesylate linked to an anti-FRα antibody or antigen-binding fragment thereof comprising a VH comprising the amino acid sequence of SEQ ID NO:99 and a VL comprising the amino acid sequence of SEQ ID NO:101. The eribulin mesylate can be linked to the antibody or antigen-binding fragment thereof via a cathepsin-B linker. The immunoconjugate can comprise 1-10, 2-5, or 4 eribulin mesylate.

The anti-FRα immunoconjugate can comprise eribulin mesylate linked to an anti-FRα antibody or antigen-binding fragment thereof comprising a heavy chain comprising the amino acid sequence of SEQ ID NO:103 and a VL comprising the amino acid sequence of SEQ ID NO:104. The eribulin mesylate can be linked to the antibody or antigen-binding fragment thereof via a cathepsin-B linker. The immunoconjugate can comprise 1-10, 2-5, or 4 eribulin mesylate.

In some aspects, an anti-FRα immunoconjugate is MORAb-202. MORAb-202 is an antibody-drug conjugate containing farletuzumab (MORAb-003) conjugated to eribulin mesylate via a cathepsin-B-cleavable linker. MORAb-202. MORAb-202 is disclosed in U.S. Published Application No. 2020/0297860, which is herein incorporated by reference in its entirety.

The anti-FRα immunoconjugate can comprise 3-aminophenyl hemiasterlin (SC209) linked to an anti-FRα antibody or antigen-binding fragment thereof comprising (a) VH CDR1, VH CDR2, and VH CDR3 comprising the amino acid sequences of SEQ ID NOs:91 or 94, 92 or 95, and 93, respectively; and (b) VL CDR1, VL CDR2, and VL CDR3 comprising the amino acid sequences of SEQ ID NOs:96-98, respectively. The anti-FRα antibody or antigen-binding fragment thereof can comprise antibody comprises one or more non-natural amino acids at sites selected from the group consisting of: HC-F404, HC-Y180, and LC-K42 according to the Kabat or EU numbering scheme of Kabat. The immunoconjugate can comprise an SC239 linker-cytotoxin. The immunoconjugate can comprise 1-10, 2-5, or 4 SC239s.

The anti-FRα immunoconjugate can comprise 3-aminophenyl hemiasterlin (SC209) linked to an anti-FRα antibody or antigen-binding fragment thereof comprising a VH comprising the amino acid sequence of SEQ ID NO:100 and a VL comprising the amino acid sequence of SEQ ID NO:102. The anti-FRα antibody or antigen-binding fragment thereof can comprise antibody comprises one or more non-natural amino acids at sites selected from the group consisting of: HC-F404, HC-Y180, and LC-K42 according to the Kabat or EU numbering scheme of Kabat. The immunoconjugate can comprise an SC239 linker-cytotoxin. The immunoconjugate can comprise 1-10, 2-5, or 4 SC239s.

In some aspects, an anti-FRα immunoconjugate is STRO-002. STRO-002 contains the anti-FolRa human IgG1 antibody (SP8166) conjugated to a cleavable drug-linker (SC239). STRO-002 is disclosed in U.S. Published Application No. 2019/0083641, which is herein incorporated by reference in its entirety.

In some aspects, for compositions (e.g., pharmaceutical compositions) comprising immunoconjugates of the first aspect (A1), or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th} specific aspects (A1-1, A1-2, A1-3, A1-4, A1-5, A1-6, A7, A8, A9, A10, A11, A12, A13, A14, or A15), the average number of the cytotoxic agent per antibody molecule (i.e., average value of q), also known as Drug-Antibody Ratio (DAR) in the composition is in the range of 1.0 to 8.0. In some aspects, DAR is in the range of 1.0 to 5.0, 1.0 to 4.0, 1.5 to 4.0, 2.0 to 4.0, 2.5 to 4.0, 1.0 to 3.4, 1.0 to 3.0, 3.0 to 4.0, 3.1 to 3.5, 3.1 to 3.7, 3.4 to 3.6, 1.5 to 2.5, 2.0 to 2.5, 1.7 to 2.3, or 1.8 to 2.2. In some aspects, the DAR is less than 4.0, less than 3.8, less than 3.6, less than 3.5, less than 3.0 or less than 2.5. In some aspects, the DAR is in the range of 3.1 to 3.7. In some aspects, the DAR is in the range of 3.1 to 3.4. In some aspects, the DAR is in the range of 3.3 to 3.7. In some aspects, the DAR is in the range of 3.5 to 3.9. In some aspects, the DAR is 3.1, 3.2, 3.3, 3.4, 3.5, 3.6, 3.7 or 3.8. In some aspects, the DAR is 3.5. In some aspects, the DAR is in the range of 1.8 to 2.0. In some aspects, the DAR is in the range of 1.7 to 1.9. In some aspects, the DAR is in the range of 1.9 to 2.1. In some aspects, the DAR is 1.9, 2.0 or 2.1. In some aspects, for the immunoconjugates comprising an anti-FRα antibody or an antigen-binding fragment thereof linked to the maytansinoid compound through one or more cysteine thiol group, the DAR is in the range of 1.5 to 2.5, 1.8 to 2.2, 1.1 to 1.9 or 1.9 to 2.1. In some aspects, the DAR is 1.8, 1.9, 2.0 or 2.1.

### B. Linkers

Any suitable linkers known in the art can be used in preparing the immunoconjugates. In some aspects, the linkers are bifunctional linkers. As used herein, the term “bifunctional linker” refers to modifying agents that possess two reactive groups; one of which is capable of reacting with a cell binding agent while the other one reacts with the maytansinoid compound to link the two moieties together. Such bifunctional crosslinkers are well known in the art (see, for example, Isalm and Dent in Bioconjugation chapter 5, p 218-363, Groves Dictionaries Inc. New York, 1999). For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups on to a cell binding agent are well known in the art (see US Patent Publication Nos. 2008/0050310, 20050169933, available from Pierce Biotechnology Inc. P.O. Box 117, Rockland, IL 61105, USA) and include, but not limited to, bis-maleimidopolyethyleneglycol (BMPEO), BM(PEO)₂, BM(PEO)₃, N-(β-maleimidopropyloxy)succinimide ester (BMPS), 7-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(β-maleimidophenyl)-butyrate (SMPB), succinimidyl-6-(β-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4-bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(γ-maleimidobutryloxy)sulfosuccinimide ester (sulfo-GMBS or sGMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(β-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with an anti-FRα antibody or antigen-binding fragment thereof. Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present disclosure. Examples of bifunctional crosslinking agents that can be used include succinimidyl-p-formyl benzoate (SFB) and succinimidyl-p-formylphenoxyacetate (SFPA).

Bifunctional crosslinking agents that enable the linkage of cell binding agent with cytotoxic compounds via disulfide bonds are known in the art and include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB or sSPDB) to introduce dithiopyridyl groups. Other bifunctional crosslinking agents that can be used to introduce disulfide groups are known in the art and are disclosed in U.S. Pat. Nos. 6,913,748, 6,716,821 and US Patent Publications 2009/0274713 and 2010/0129314, each of which is herein incorporated by reference in its entirety. Alternatively, crosslinking agents such as 2-iminothiolane, homocysteine thiolactone or S-acetylsuccinic anhydride that introduce thiol groups can also be used.

In some aspects, a linker is a cathepsin-B linker.

### C. Cytotoxic Agents

The cytotoxic agent in the immunoconjugate can be any compound that results in the death of a cell, or induces cell death, or in some manner decreases cell viability (e.g., tubulin-acting agents, DNA alkylating agents, DNA crosslinking agents, DNA topoisomerase inhibiting agents), and includes, for example, maytansinoids and maytansinoid analogs, benzodiazepines, indolinobenzodiazepines, camptothecins, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin. In some aspects, the cytotoxic agents are maytansinoids and maytansinoids analogs.

Examples of suitable maytansinoids include esters of maytansinol and maytansinol analogs. Included are any drugs that inhibit microtubule formation and that are highly toxic to mammalian cells, as are maytansinol and maytansinol analogs

Exemplary cytotoxic agents were described previously in WO 2018/160539 A1 and WO 2011/106528, each of which is herein incorporated by reference in its entirety.

The immunoconjugates provided herein can comprise a maytansinoid compound represented by the following formula:

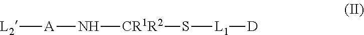

or a pharmaceutically acceptable salt thereof, wherein:
L₂′ is represented by the following structural formulas:

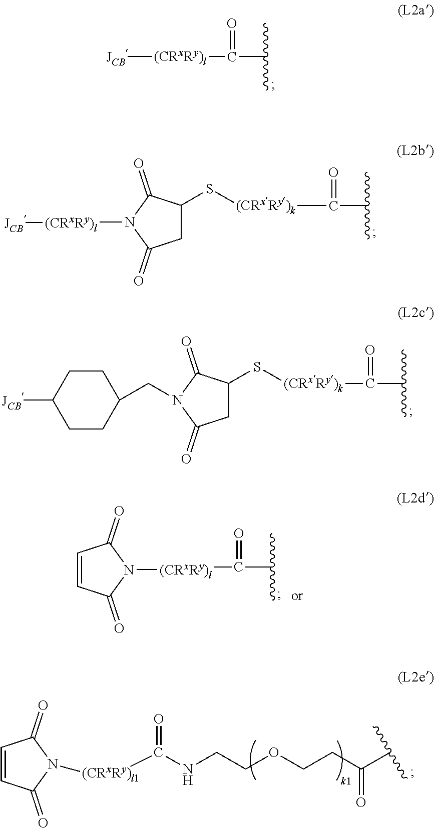

wherein:
R^{x}, R^{y}, R^{x′} and R^{y′}, for each occurrence, are independently H, —OH, halogen, —O—(C₁₋₄ alkyl), —SO₃H, —NR₄₀R₄₁R₄₂⁺, or a C₁₋₄ alkyl optionally substituted with —OH, halogen, —SO₃H or NR₄₀R₄₁R₄₂⁺, wherein R₄₀, R₄₁ and R₄₂ are each independently H or a C₁₋₄ alkyl;
l and k are each independently an integer from 1 to 10;
J_{CB}′ is —C(═O)OH or —COE, wherein —COE is a reactive ester;
A is an amino acid or a peptide comprising 2 to 20 amino acids;
R¹ and R² are each independently H or a C₁₋₃alkyl;
L₁ is represented by the following formula:

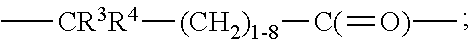

wherein R³ and R⁴ are each independently H or Me, and the —C(═O)— moiety in L₁ is connected to D;
D is represented by the following formula:

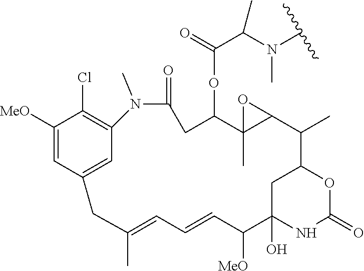 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

and
q is an integer from 1 to 20. In some aspects q is an integer from 1 to 10. In some aspects q is an integer from 2 to 5. In some aspects, q is an integer from 3 to 4.

In some aspects, the maytansinoid is represented by the following formula:

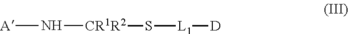

or a pharmaceutically acceptable salt thereof, wherein:
A′ is an amino acid or a peptide comprising 2 to 20 amino acids (i.e., A-NH₂);
R¹ and R² are each independently H or a C₁₋₃alkyl;
L₁ is —CR³R₄—(CH₂)₁₋₈—C(═O)—; R³ and R⁴ are each independently H or Me;
D is represented by the following formula:

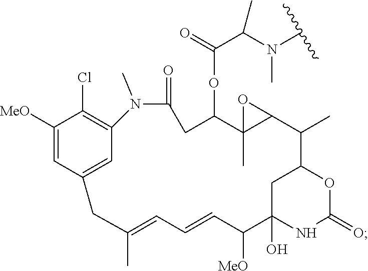 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

and
q is an integer from 1 to 20. In some aspects q is an integer from 1 to 10. In some aspects q is an integer from 2 to 5. In some aspects, q is an integer from 3 to 4.

In some aspects, the maytansinoid is represented by the following formula:

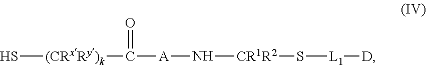

or a pharmaceutically acceptable salt thereof, wherein:
R^{x′} and R^{y′}, for each occurrence, are independently H, —OH, halogen, —O—(C₁₋₄ alkyl), —SO₃H, —NR₄₀R₄₁R₄₂, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
k is an integer from 1 to 10
A is an amino acid residue or a peptide comprising 2 to 20 amino acid residues;
R¹ and R² are each independently H or a C₁₋₃alkyl;
L₁ is —CR³R₄—(CH₂)₁₋₈—C(═O)—; R³ and R⁴ are each independently H or Me;
D is represented by the following formula:

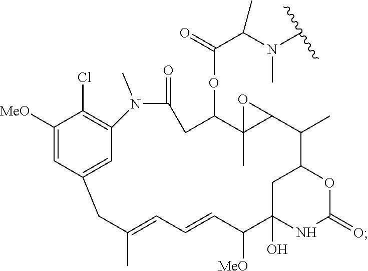 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

and
q is an integer from 1 to 20. In some aspects q is an integer from 1 to 10. In some aspects q is an integer from 2 to 5. In some aspects, q is an integer from 3 to 4.

In some aspects, for maytansinoid compounds of formulas (II), (III) or (IV), the variables are as described in the first aspect (A1), or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th} specific aspects (A1-1, A1-2, A1-3, A1-4, A1-5, A1-6, A7, A8, A9, A10, A11, A12, A13, A14, or A15).

In some aspects, the maytansinoid compound is represented by the following formula:

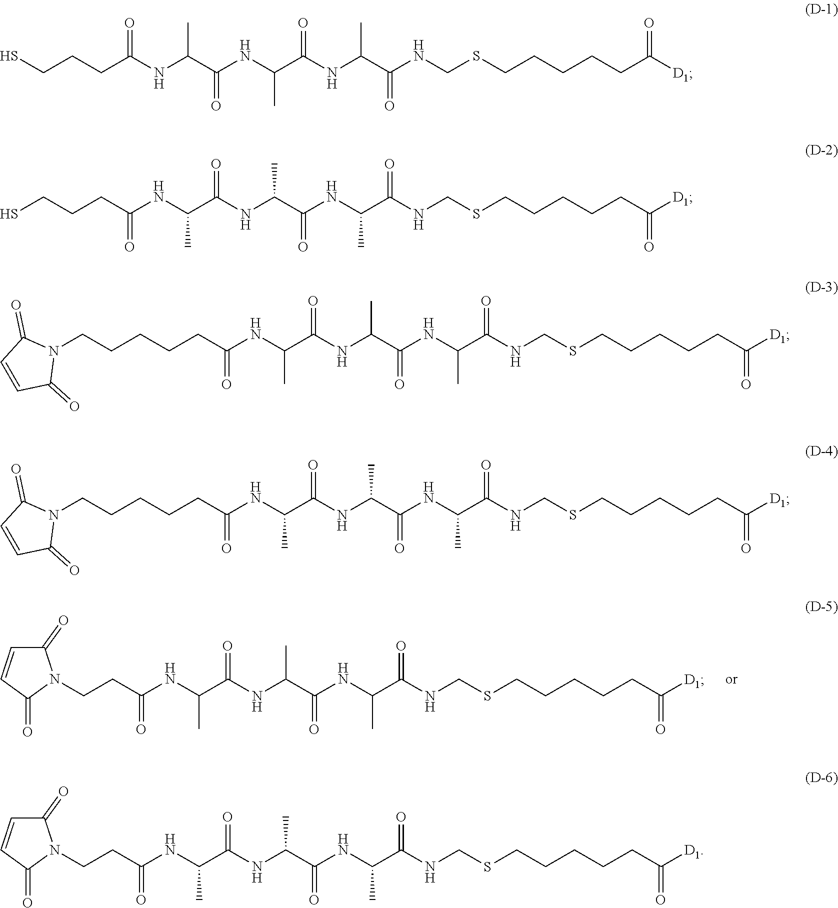

Additional examples of suitable maytansinol esters include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 7,276,497 and 7,473,796. In addition, several descriptions for producing such antibody-maytansinoid conjugates are provided in U.S. Pat. Nos. 6,333,410, 6,441,163, 6,716,821, and 7,368,565, each of which is herein incorporated by reference in its entirety.

In some aspects, the immunoconjugate comprises N^{2′}-deacetyl-N^{2′}-(3-mercapto-1-oxopropyl)-maytansine (DM1), N^{2′}-deacetyl-N-^{2′}(4-mercapto-1-oxopentyl)-maytansine (termed DM3), N^{2′}-deacetyl-N^{2′}-(4-mercapto-4-methyl-1-oxopentyl) maytansine (DM4), both of which were previously described in PCT Application Publication No. WO 2011/106528 A1 and U.S. Pat. No. 8,557,966 B2, each of which is herein incorporated by reference in its entirety.

In some aspects, an immunoconjugate comprises 3-aminophenyl hemiasterlin (SC209).

In some aspects, an immunoconjugate comprises eribulin mesylate.

### D. Drug Conjugation

The immunoconjugates comprising an anti-FRα-binding antibody or antigen-binding fragment thereof covalently linked to a cytotoxic agent (e.g., maytansinoid) described herein can be prepared according to any suitable methods known in the art.

In some aspects, the immunoconjugates of the first aspect (A1) can be prepared by a first method comprising the steps of reacting the anti-FRα antibody or antigen-binding fragment thereof with the maytansinoid compound of formula (II).

In some aspects, the immunoconjugates of the first aspect (A1) can be prepared by a second method comprising the steps of:
(a) reacting the maytansinoid compound of formula (III) or (IV) with a linker compound described herein to form a cytotoxic agent-maytansinoid compound having an amine-reactive group or a thiol-reactive group bound thereto (e.g., compound of formula (II)) that can be covalently linked to the anti-FRα antibody or antigen-binding fragment thereof; and
(b) reacting the anti-FRα antibody or antigen-binding fragment thereof with the maytansinoid-linker compound to form the immunoconjugate.

In some aspects, the immunoconjugates of the first aspect (A1) can be prepared by a third method comprising the steps of.
(a) reacting the anti-FRα antibody or antigen-binding fragment thereof with a linker compound described herein to form a modified anti-FRα antibody or antigen-binding fragment thereof having an amine-reactive group or a thiol-reactive group bound thereto that can be covalently linked to the maytansinoid compound of formula (III) or (IV); and
(b) reacting the modified anti-FRα antibody or antigen-binding fragment thereof with the maytansinoid compound of formula (III) or (IV) to form the immunoconjugate.

In some aspects, for the second, third or fourth methods described above, the linker compound is represented by any one of the formula (a1L)-(a10L):

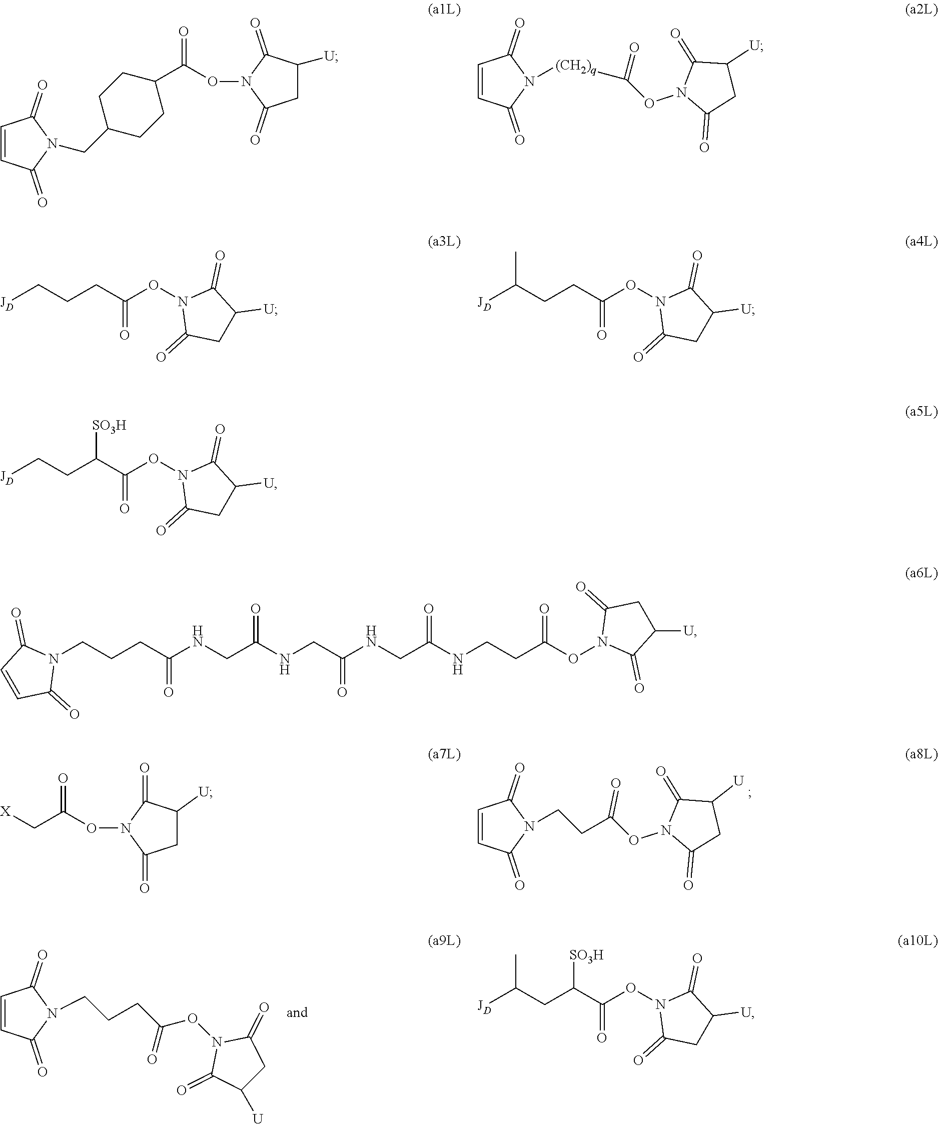

wherein X is halogen; J_{D}-SH, or —SSR^{d}; R^{d} is phenyl, nitrophenyl, dinitrophenyl, carboxynitrophenyl, pyridyl or nitropyridyl; R⁹ is an alkyl; and U is —H or SO₃H or a pharmaceutically acceptable salt thereof.

In some aspects, the linker compound is GMBS or sulfo-GMBS (or sGMBS) represented by represented by formula (a9L), wherein U is —H or SO₃H or a pharmaceutically acceptable salt thereof.

In some aspects, an immunoconjugate is represented by the following formula:

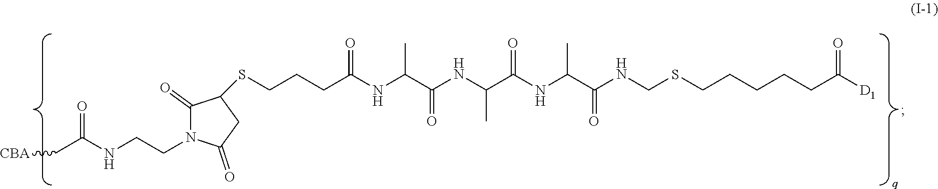

and the immunoconjugate is prepared by the second, third or fourth method described above, wherein the linker compound is GMBS or sulfo-GMBS represented by represented by formula (a9L), wherein U is —H or SO₃H or a pharmaceutically acceptable salt thereof; and the maytansinoid compound is represented by formula (D-1) described above. In some aspects, the immunoconjugate of formula (I-1) is prepared by reacting the maytansinoid compound of formula (D-1) with the linker compound GMBS or sulfo-GMBS to form a maytansinoid-linker compound, followed by reacting the anti-FRα antibody or antigen-binding fragment thereof with the maytansinoid-linker compound. In some aspects, the maytansinoid linker compound is not purified before reacting with the anti-FRα antibody or antigen-binding fragment thereof.

In some aspects, the immunoconjugate is represented by the following formula:

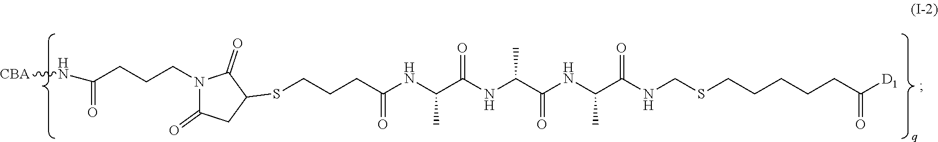

and the immunoconjugate can be prepared by the second, third or fourth method described above, wherein the linker compound is GMBS or sulfo-GMBS represented by represented by formula (a9L), wherein U is —H or SO₃H or a pharmaceutically acceptable salt thereof; and the maytansinoid compound is represented by formula (D-2) described above. In some aspects, the immunoconjugate of formula (I-2) is prepared by reacting the maytansinoid compound of formula (D-2) with the linker compound GMB S or sulfo-GMB S to form a maytansinoid-linker compound, followed by reacting the anti-FRα antibody or antigen-binding fragment thereof with the maytansinoid-linker compound. In some aspects, the maytansinoid linker compound is not purified before reacting with the anti-FRα antibody or antigen-binding fragment thereof. In some aspects, the immunoconjugate is represented by the following formula:

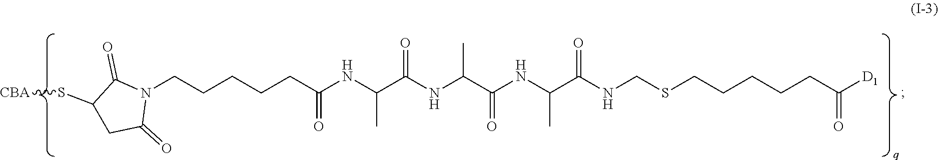

and the immunoconjugate is prepared according to the first method described above by reacting the anti-FRα antibody or antigen-binding fragment thereof with the maytansinoid compound of formula (D-3) described above.

In some aspects, the immunoconjugate is represented by the following formula:

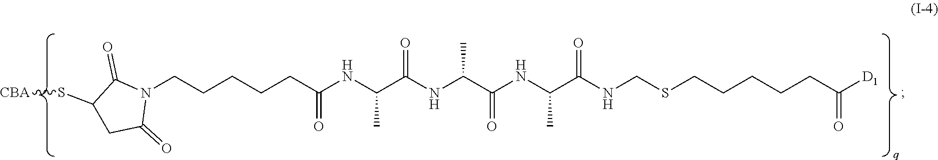

and the immunoconjugate is prepared according to the first method described above by reacting the anti-FRα antibody or antigen-binding fragment thereof with the maytansinoid compound of formula (D-4) described above.

In some aspects, the immunoconjugate is represented by the following formula:

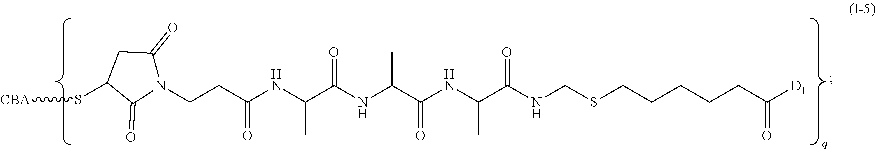

and the immunoconjugate is prepared according to the first method described above by reacting the anti-FRα antibody or an antigen-binding fragment thereof with the maytansinoid compound of formula (D-5) described above.

In some aspects, the immunoconjugate is represented by the following formula:

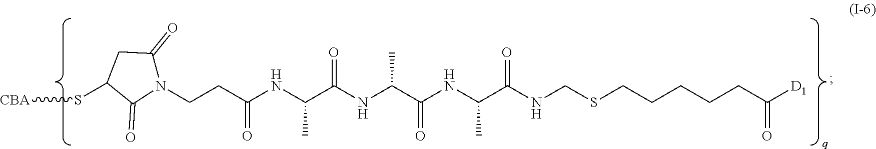

and the immunoconjugate is prepared according to the first method described above by reacting the anti-FRα antibody or antigen-binding fragment thereof with the maytansinoid compound of formula (D-6) described above.

In some aspects, the immunoconjugates represented by formulas I-3 through I-6 disclosed above are prepared according to the methods described in U.S. Provisional Application 62/821,707 filed on Mar. 21, 2019 and related U.S. application Ser. No. 16/825,127.

In some aspects, the immunoconjugates prepared by any methods described above is subject to a purification step. In this regard, the immunoconjugate can be purified from the other components of the mixture using tangential flow filtration (TFF), non-adsorptive chromatography, adsorptive chromatography, adsorptive filtration, selective precipitation, or any other suitable purification process, as well as combinations thereof.

In some aspects, the immunoconjugate is purified using a single purification step (e.g., TFF). In some aspects, the conjugate is purified and exchanged into the appropriate formulation using a single purification step (e.g., TFF). In some aspects, the immunoconjugate is purified using two sequential purification steps. For example, the immunoconjugate can be first purified by selective precipitation, adsorptive filtration, absorptive chromatography or non-absorptive chromatography, followed by purification with TFF. One of ordinary skill in the art will appreciate that purification of the immunoconjugate enables the isolation of a stable conjugate comprising the cell-binding agent chemically coupled to the cytotoxic agent.

Any suitable TFF systems may be utilized for purification, including a Pellicon type system (Millipore, Billerica, Mass.), a Sartocon Cassette system (Sartorius AG, Edgewood, N.Y.), and a Centrasette type system (Pall Corp., East Hills, N.Y.)

Any suitable adsorptive chromatography resin may be utilized for purification. Adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.) Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g., Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

### VI. Compositions and Kits

Provided herein are compositions comprising an-anti FRα active agent (e.g., immunoconjugate, antibody, or antigen-binding fragment thereof) described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed.

A pharmaceutical composition may be formulated for a particular route of administration to a subject. For example, a pharmaceutical composition can be formulated for parenteral, e.g., intravenous, administration. The compositions to be used for in vivo administration can be sterile. This is readily accomplished by filtration through, e.g., sterile filtration membranes.

The pharmaceutical compositions described herein are, in some aspects, for use as a medicament. Pharmaceutical compositions described herein can be useful in treating a condition such as cancer. Examples of cancer that can be treated as described herein include, but are not limited to, carcinoma, lymphoma, blastoma, sarcoma, and leukemia. More particular examples of such cancers include fallopian tube cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

A pharmaceutical composition provided herein can comprise anti-FRα immunoconjugates and the pharmaceutical composition (immunoconjugates in the pharmaceutical composition) can have an average of 1 to 20 drugs per anti-FRα antibody or antigen-binding fragment thereof. In some aspects, a pharmaceutical composition comprises an average of 1 to 10 drugs per anti-FRα antibody or antigen-binding fragment thereof. In some aspects, a pharmaceutical composition comprises an average of 2 to 5 drugs per anti-FRα antibody or antigen-binding fragment thereof. In some aspects, a pharmaceutical composition comprises an average of 3 to 4 drugs per anti-FRα antibody or antigen-binding fragment thereof. In some aspects, a pharmaceutical composition comprises about 3.5 drugs per anti-FRα antibody or antigen-binding fragment thereof.

Provided herein are also kits comprising reagents for the detection of soluble FRα and an anti-FRα active agent or instructions to administer an anti-FRα active agent if soluble FRα or a high level of soluble FRα is detected. The reagents for detection of soluble FRα can comprise (a) a first reagent, which can be an immunocapture reagent, which binds to FRα; and (b) a digestion reagent, which can digest captured FRα into peptides. As provided elsewhere herein, the immunocapture reagent can be an anti-FRα antibody or antigen-binding fragment thereof, including, for example, the FR1-9 antibody or an antigen-binding fragment thereof, the FR1-13 antibody or an antigen-binding fragment thereof, an antibody or an antigen-binding fragment thereof comprising the CDRs of the FR1-9 or FR1-13 antibody, or an antibody or antigen-binding fragment thereof comprising the VH and/or VL of the FR1-9 or FR1-13 antibody.

The kit can further comprise at least one peptide derived from FRα. The peptide can be used as a standard in liquid chromatography-mass spectrometry analyses. In some aspects, the kit contains a peptide comprising the sequence of SEQ ID NO:68. In some aspects, the kit contains a peptide comprising the sequence of SEQ ID NO:69. In some aspects, the kit contains a peptide comprising the sequence of SEQ ID NO:70. In some aspects, the kit contains a peptide comprising the sequence of SEQ ID NO:71. In some aspects, the kit comprises four signature peptides which can be used as a standard in liquid chromatography-mass spectrometry analyses. In some aspects, the four signature peptides consist of 1) a peptide comprising the sequence of SEQ ID NO:68; 2) a peptide comprising the sequence of SEQ ID NO:69; 3) a peptide comprising the sequence of SEQ ID NO:70; and 4) a peptide comprising the sequence of SEQ ID NO:71.

The kit can further comprise a solid support for the capture reagents, which can be provided as a separate element or upon which the capture reagents are already immobilized. Hence, the capture antibodies or antigen-binding fragments thereof in the kit can be immobilized on a solid support, or they can be immobilized on such support that is included with the kit or provided separately from the kit. In some aspects, the solid support comprises a mass spectrometric immunoassay (MSIA) microcolumn. In some aspects, the solid support comprises magnetic beads. In some aspects, the solid support is coated with streptavidin. In some aspects, the immunocapture reagent is attached to the solid support through a biotin-streptavidin interaction.

### VII. Cancers for Treatment

As provided herein, cancers in patients with a soluble FRα level equal to or greater than a target soluble FRα level can be treated using anti-FRα active agents. In certain aspects, the cancer is a cancer including, but are not limited to, fallopian tube cancer, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer (TNBC)), colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

More particular examples of such cancers include ovarian cancer, epithelial ovarian cancer, ovarian primary peritoneal cancer, or fallopian tube cancer. In some aspects, the cancer is ovarian cancer. In some aspects, the ovarian cancer is epithelial ovarian cancer (EOC). In some aspects, the ovarian cancer (e.g., an EOC) is platinum resistant, relapsed, or refractory. In some aspects, the cancer is peritoneal cancer. In some aspects, the peritoneal cancer is primary peritoneal cancer. In some aspects, the cancer is endometrial cancer. In some aspects, the endometrial cancer is serous endometrial cancer. In some aspects, cancer is lung cancer. In some aspects, the lung cancer is non-small cell lung cancer (NSCLC). In some aspects, the lung cancer is lung cancer is adenocarcinoma or bronchioloalveolar carcinoma. In some aspects, the cancer is uterine cancer.

In some aspects, the cancer is platinum refractory. In some aspects, the cancer is primary platinum refractory. In some aspects, the cancer is platinum sensitive.

In some aspects, the cancer is a metastatic or advanced cancer.

In certain aspects, a cancer for treatment with IMGN151 is resistant to treatment with IMGN853.

As demonstrated herein, soluble FRα levels can be in independent a predictor of responsiveness to FRα-targeted therapy (e.g., independent of membrane FRα scores as measured by immunohistochemistry (IHC) (see e.g., Examples 2 and 3). Accordingly, in some aspects provided herein, an IHC score has not been obtained for the tumor. Methods of detecting membrane FRα by IHC are known in the art and are provided, for example, in WO 2012/135675, and WO 2015/031815, each of which is herein incorporated by reference in its entirety.

FRα can be detected by IHC using anti-FRα antibodies and antigen-binding fragments thereof. Anti-FRα antibodies and antigen-binding fragments thereof useful in the detection of FRα by IHC can be called IHC FRα-detection antibodies or antigen-binding fragments thereof. IHC FRα-detection antibodies or antigen-binding fragments thereof include, e.g., the 2.1 antibody described in Section II, above. IHC FRα-detection antibodies or antigen-binding fragments thereof also include, e.g., antibodies and antigen-binding fragments thereof that comprise the six CDRs (i.e., the VH-CDR1, VH-CDR2, VH-CDR3, VL-CDR1, VL-CDR2, and VL-CDR3) of 2.1, or the VH and/or VL of 2.1.

In some aspects, an IHC FRα-detection antibody or antigen-binding fragment thereof comprises (a) VH CDR1, VH CDR2, and VH CDR3 comprising the amino acid sequences of SEQ ID NOs:30-32, respectively; and (b) VL CDR1, VL CDR2, and VL CDR3 comprising the amino acid sequences of SEQ ID NOs:33-35, respectively.

In some aspects, an IHC FRα-detection antibody or antigen-binding fragment thereof comprises (a) a variable heavy chain (VH) comprising the amino acid sequence of SEQ ID NO:41 and/or (b) a variable light chain (VL) comprising the amino acid sequence of SEQ ID NO:47.

In some aspects, an IHC FRα-detection antibody or antigen-binding fragment thereof comprises (a) a heavy chain (HC) comprising the amino acid sequence of SEQ ID NO:53 and/or (b) a light chain (CL) comprising the amino acid sequence of SEQ ID NO:59.

In some aspects, membrane FRα protein measured by IHC is given a staining intensity score and/or a staining uniformity score by comparison to controls (e.g., calibrated controls) exhibiting defined scores (e.g. an intensity score of 3⁺ is given to the test sample if the intensity is comparable to the level 3⁺ calibrated control or an intensity of 2+ (moderate) is given to the test sample if the intensity is comparable to the level 2+ calibrated control). A staining uniformity is based on the percent of stained cells.

In some aspects, a tumor sample obtained from the patient does not have at least 75% of cells having with percent staining (PS)2+ staining intensity. In some aspects, a tumor sample obtained from the patient does not have at least 50% of cells having with PS2+ intensity. In some aspects, the IHC staining and intensity has been determined prior to the administration. As demonstrated herein, such patients can be successfully treated with an anti-FRα active agent (e.g., immunoconjugate) because soluble FRα predicts the efficacy of such active agents independently of membrane FRα IHC scores.

In some aspects, a tumor sample obtained from the patient has at least 75% of cells with PS2+ staining intensity. In some aspects, a tumor sample obtained from the patient has at least 50% of cells with PS2+ staining intensity. In some aspects, the IHC staining and intensity has been determined prior to the administration.

In some aspects, the subject is a human.

Administration of the anti-FRα active agent can be parenteral, including intravenous, administration.

The amount of anti-FRα active agent (e.g., immunoconjugate, antibody or antigen-binding fragment thereof) or composition which will be effective in the treatment of a condition will depend on the nature of the disease. The precise dose to be employed in a composition will also depend on the route of administration, and the seriousness of the disease.

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### Examples

It is understood that the examples and aspects described herein are for illustrative purposes only and that various modifications or changes in light thereof will be suggested to persons skilled in the art and are to be included within the spirit and purview of this application.

### Example 1. Soluble FRα Levels Predict Efficacy of FRα-Targeted Therapy

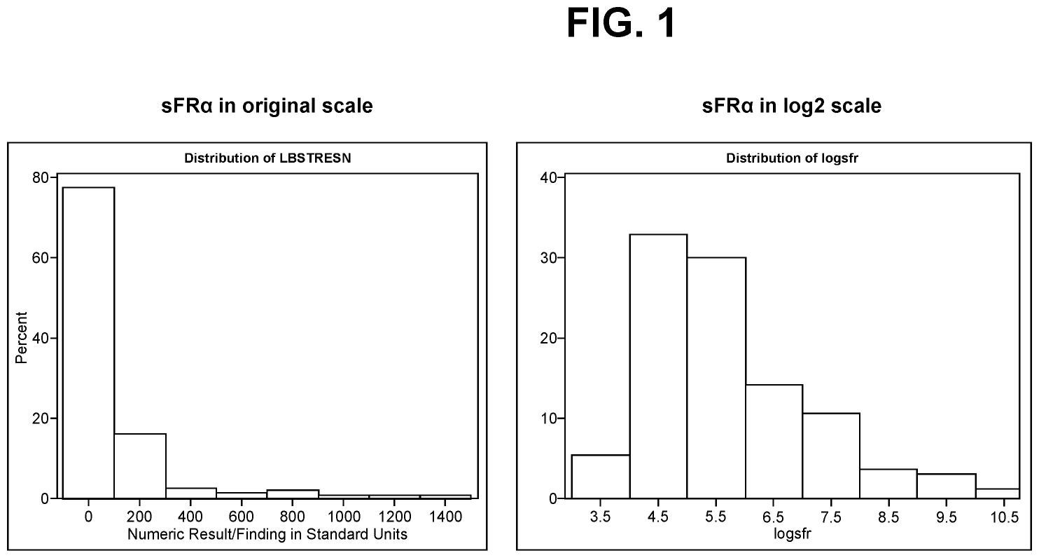

Levels of soluble FRα were detected in patients enrolled in a Phase 3 clinical trial (FORWARD I) involving administration of the anti-FRα immunoconjugate IMGN853 (mirvetuximab soravtansine). The trial was designed to compare the safety and efficacy of IMGN853 to that of selected single-agent chemotherapy (Investigator's choice (IC)) in women with platinum-resistant advanced epithelial ovarian cancer (EOC), primary peritoneal cancer, and/or fallopian tube cancer. All of the enrolled patients had a FRα-positive tumor as determined by 10× immunohistochemistry (IHC). (These patients IHC scores were subsequently reevaluated using PS2 scoring.) The soluble FRα levels were determined by immunocapturing soluble FRα, digesting the soluble FRα, and performing liquid chromatography-mass spectrometry (LC/MS) analysis on the digested soluble FRα (as disclosed in U.S. Published Application No. 2020/0284810, which is herein incorporated by reference in its entirety). The distribution of soluble FRα levels in the patients is shown in FIG. 1.

The patients were randomized and treated with either IMGN853 administered at 6 mg/kg adjusted ideal body weight (AIBW) once every three weeks (Q3W) or with paclitaxel (80 mg/m² weekly), pegylated liposomal doxorubicin (40 mg/m² once every 4 weeks (Q4W)), or topotecan (4 mg/m² on Days 1, 8, and 15 Q4W or 1.25 mg/m² on Days 1-5 Q3W). The progression free survival (PFS), objective response rate (ORR) per RECIST 1.1, and overall survival (OS) as measured from the date of randomization until the date of death were measured for up to 2 years. PFS and ORR were both measured by a blinded independent review committee (BIRC) and by investigators (INV).

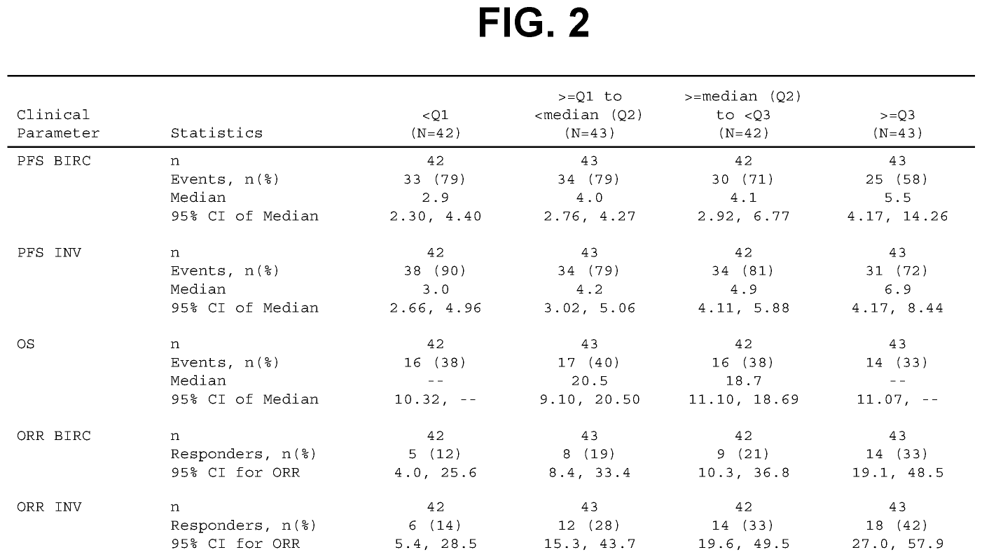

The patients were divided into four quartiles based on levels of soluble FRα. Patients in the first quartile “Q1” had the lowest levels of soluble FRα, and patients in the fourth quartile “Q4” had the highest levels of soluble FRα. The efficacy of IMGN853 treatment in each of these quartiles was determined. The results are shown in FIG. 2. The median PFS time in Q4 was 2.6 months longer than the median PFS time in Q1 (5.5 months vs. 2.9 months) as measured by BIRC and 3.9 months longer as measured by INV (6.9 months vs. 3.0 months). In addition, the overall response rate was 33% in Q4 as compared to only 12% in Q1 based on BIRC and 42% in Q4 as compared to only 14% in Q1 based on INV.

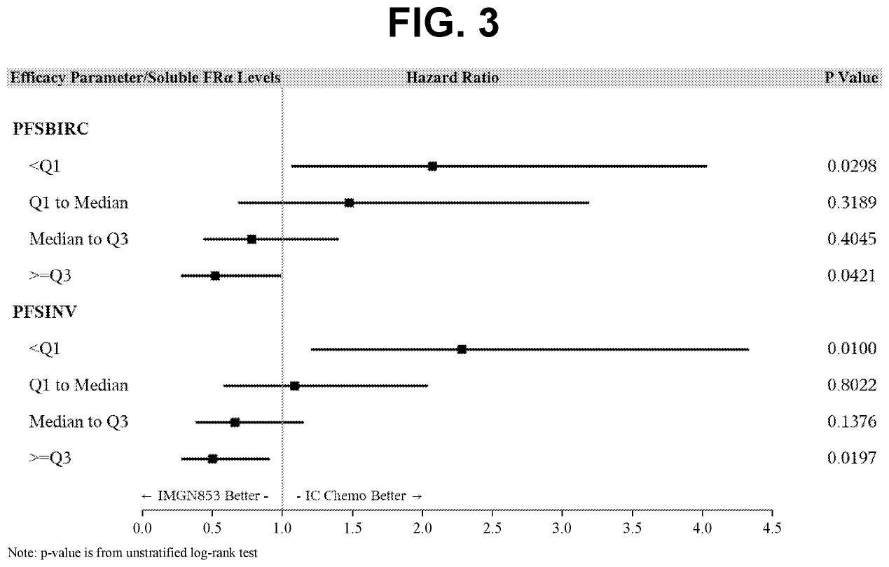

The relative efficacy of IMGN853 and chemotherapy was also compared in each of the quartiles (FIG. 3). Patients in the two quartiles with higher levels of soluble FRα (Q3 and Q4) had longer PFS times when treated with IMGN853 than when treated with chemotherapy. In contrast, patients in the two quartiles with lower levels of soluble FRα (Q1 and Q2) had longer PFS times when treated with chemotherapy than when treated with IMGN853.

These results indicate that surprisingly, higher soluble FRα was associated with improved responses (PFS and ORR) to IMGN853. Accordingly, comparison of a patient's soluble FRα level with a target soluble FRα level can be used as an independent predictor of the likelihood of a patient's cancer to respond to an anti-FRα active agent: a soluble FRα level in a patient that is equal to or greater than the target level indicates the patient's cancer is likely to respond to an anti-FRα active agent such as IMGN853 or IMGN151.

### Example 2. Soluble FRα Levels were not Significantly Correlated with Membrane FRα IHC Levels

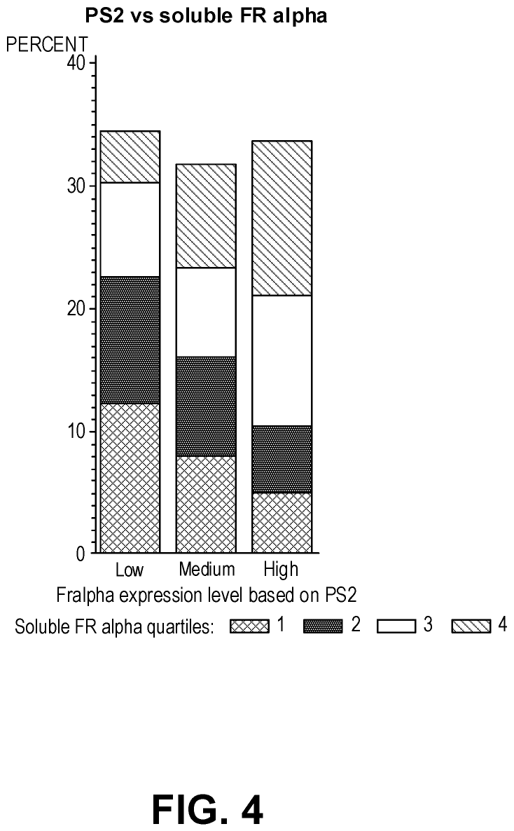

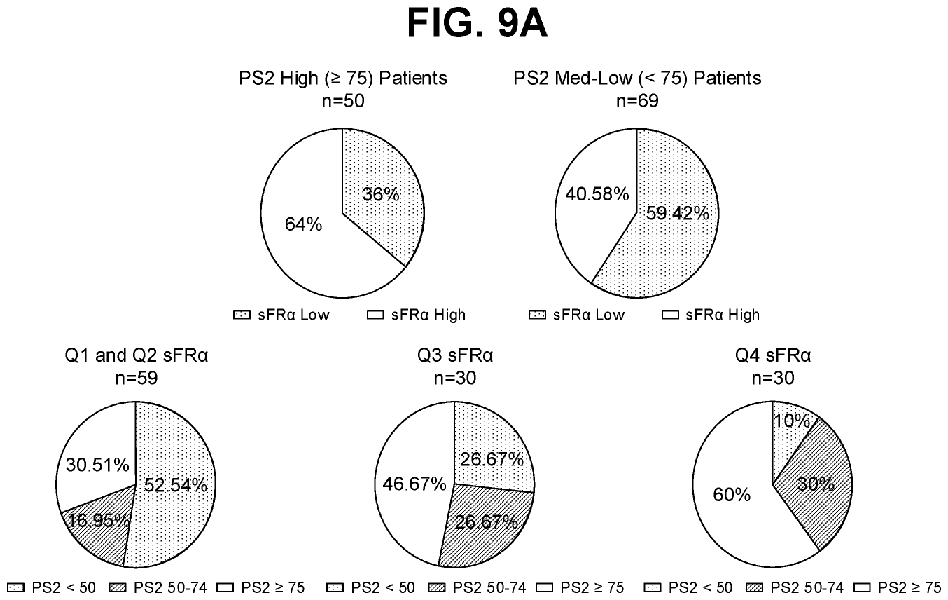

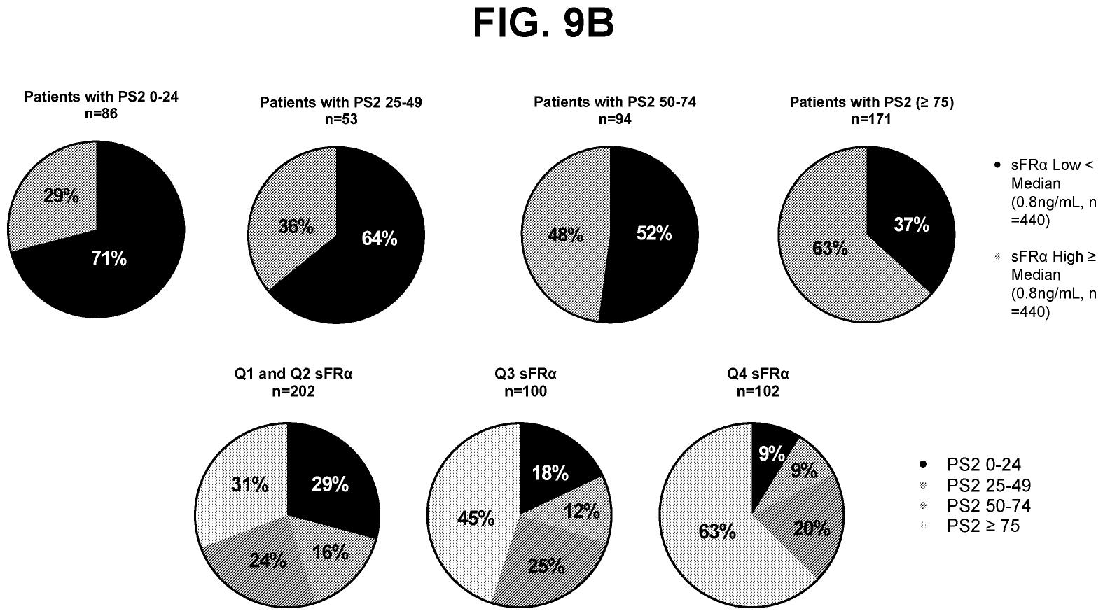

It has previously been shown that increased IHC levels of FRα in a tumor were associated with increased responsiveness to IMGN853. Therefore, the relationship between levels of soluble FRα and tumor levels of membrane FRα as measured by IHC was examined. FIG. 4 shows the percent of patients with low (less than 50% of cells with FRα membrane staining with ≥2+ intensity), medium (50-74% of cells with FRα membrane staining with ≥2+ intensity), and high (≥75% of cells with FRα membrane staining with ≥2+ intensity) membrane FRα as measured by IHC that were in each quartile of soluble FRα levels. The results demonstrate that soluble FRα and tumor levels of membrane FRα as measured by IHC were not significantly correlated. For example, patients in the highest quartile (Q4) for soluble FRα could have low, medium, or high membrane FRα as measured by IHC. (FIG. 9A.) Similarly, patients in the lowest quartile (Q1) for soluble FRα could also have low, medium, or high membrane FRα as measured by IHC. (FIG. 9A.) A similar analysis was conducted with a larger group of patients and confirmed that patients in the highest quartile (Q4) for soluble FRα could have PS2 0-24, PS2 25-49, PS250-74, or PS2≥75 membrane FRα as measured by IHC. (FIG. 9B.) Similarly, patients in the lowest quartiles (Q1 and Q2) for soluble FRα could also have PS2 0-24, PS2 25-49, PS250-74, or PS2≥75 membrane FRα as measured by IHC. (FIG. 9B.)

Moreover, partial responses and/or complete responses (as measured by BIRC and/or INV) were observed in patients treated with IMGN853 who did not have high (≥75% of cells with FRα membrane staining with ≥2+ intensity) IHC scores using PS2 staining but who had soluble FRα levels equal to or above a target soluble FRα level (e.g., soluble FRα levels in Q3 or Q4).

Therefore, soluble FRα levels represent an independent predictor of responsiveness to FRα-targeted therapy.

### Example 3. Soluble FRα Distribution was Similar in FORWARD I and MIRASOL Trials and Soluble FRα Serum Concentrations do not Significantly Correlate with IHC FRα Scores

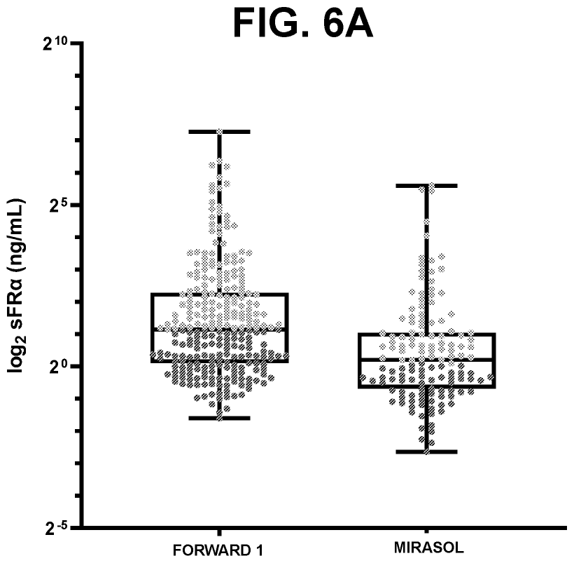

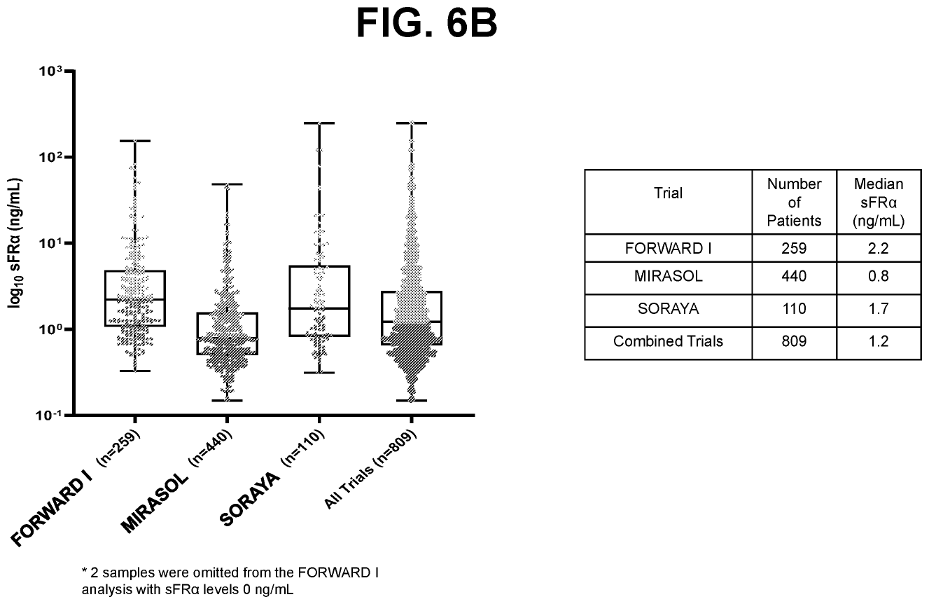

Levels of soluble FRα were detected in patients screened for a Phase 3 clinical trial (MIRSASOL) using IMGN853. The trial was designed to compare the progression free survival (PFS) of patients randomized to receive IMGN853 to that of selected single-agent chemotherapy (Investigator's choice (IC)) in women with platinum-resistant advanced epithelial ovarian cancer (EOC), primary peritoneal cancer, and/or fallopian tube cancer. Serum samples were collected from patients who were screened for MIRASOL, and soluble FRα levels were measured using LC-MS, regardless of the patient's enrollment in the study. Soluble FRα levels were compared to levels observed in patients enrolled in the FORWARD I trial. Despite the FORWARD I trial representing a more FRα-enriched population as measured by IHC, similar soluble FRα distributions were observed between the two trials (FIG. 6A). The soluble FRα levels for 135 patients who were screened for the MIRASOL trial (as shown in FIG. 6A) are also shown in Table 12, and the soluble FRα levels for the FORWARD I patients (as shown in FIG. 6A) are shown in Table 13.

TABLE 12
Soluble FRα levels for 135 patients who
were screened for the MIRASOL trial in FIG. 6

| Quartile | Soluble FRα (ng/mL) | Log2 Soluble FRα |
|---|---|---|
| Min | 0.16038 | −2.640433851 |
| 25% | 0.6588 | −0.60208754 |
| Median | 1.1529 | 0.205267382 |
| 75% | 2.05335 | 1.03797956 |
| Max | 48.33 | 5.59484709 |

TABLE 13
Soluble FRα levels for FORWARD I patients in FIG. 6

| Quartile | Soluble FRα (ng/mL) | Log2 Soluble FRα |
|---|---|---|
| Min | 0.32839 | −1.60652 |
| 25th | 1.085071 | 0.117676 |
| 50th | 2.215005 | 1.14731 |
| 75th | 4.825695 | 2.270709 |
| Max | 154.0487 | 7.267243 |

The levels of soluble FRα from a larger group of patients (440 patients for the MIRASOL trial and 250 patients for FORWARD I) are shown in FIG. 6B. An additional clinical trail called SORAYA was conducted in FRα˜-high (by IHC) platinum-resistant ovarian cancers that been previously treated with Avastin® (bevacizumab). The levels of soluble FRα in patients from the SORAYA trial and in patients from the three clinical trials (i.e., MIRASOL, FORWARD I, and SORAYA) are also shown in FIG. 6B.

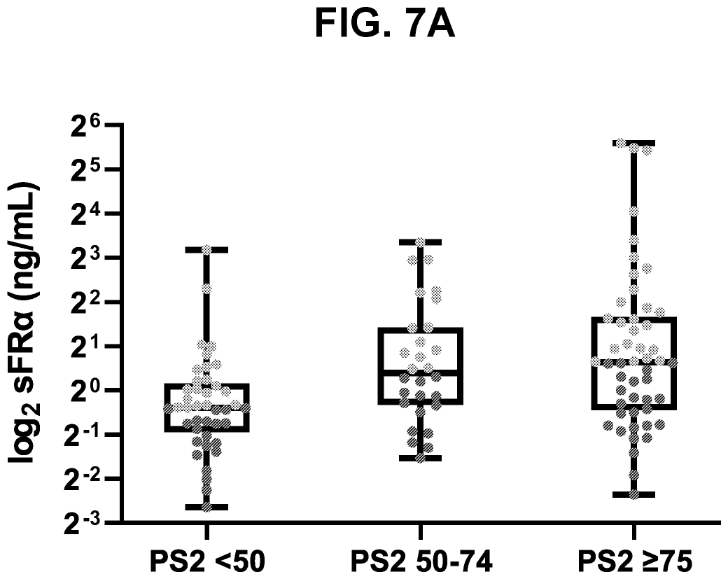

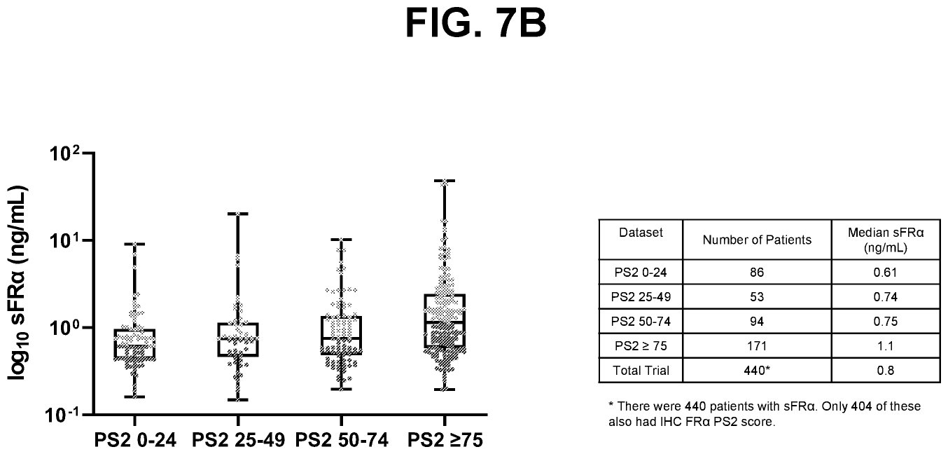

Soluble FRα data points from patients screened for the MIRASOL trial were divided into three groups based on IHC FRα˜ PS2 scores. Similar soluble FRα distributions were observed regardless of surface tumor FRα˜ expression (FIG. 7A). The soluble FRα levels for the 119 patients in the MIRASOL trial with an FRα˜-positive tumor as determined by PS2 scoring (as shown in FIG. 7A) are also shown in Tables 14A-14C.

TABLE 14A
Soluble FRα levels for MIRASOL patients
with IHC PS2 Scores <50

| Quartile | Soluble FRα (ng/mL) | Log2 Soluble FRα |
|---|---|---|
| Min | 0.16038 | −2.64043 |
| 25% | 0.5454 | −0.87461 |
| Median | 0.7614 | −0.39327 |
| 75% | 1.0719 | 0.10017 |
| Max | 9.018 | 3.172808 |

TABLE 14B
Soluble FRα levels for MIRASOL
patients with IHC PS2 Scores 50-74

| Quartile | Soluble FRα (ng/mL) | Log2 Soluble FRα |
|---|---|---|
| Min | 0.3456 | −1.53282 |
| 25% | 0.8127 | −0.29942 |
| Median | 1.3149 | 0.392024 |
| 75% | 2.665575 | 1.414443 |
| Max | 10.206 | 3.351346 |

TABLE 14C
Soluble FRα levels for MIRASOL patients
with IHC PS2 Scores >75

| Quartile | Soluble FRα (ng/mL) | Log2 Soluble FRα |
|---|---|---|
| Min | 0.19467 | −2.3609 |
| 25% | 0.7614 | −0.39423 |
| Median | 1.5498 | 0.632003 |
| 75% | 3.07125 | 1.618815 |
| Max | 48.33 | 5.594847 |

Soluble FRα data points from a larger group of patients for the MIRASOL trial were divided into four groups based on IHC FRα PS2 scores, and the results are shown in FIG. 7B.

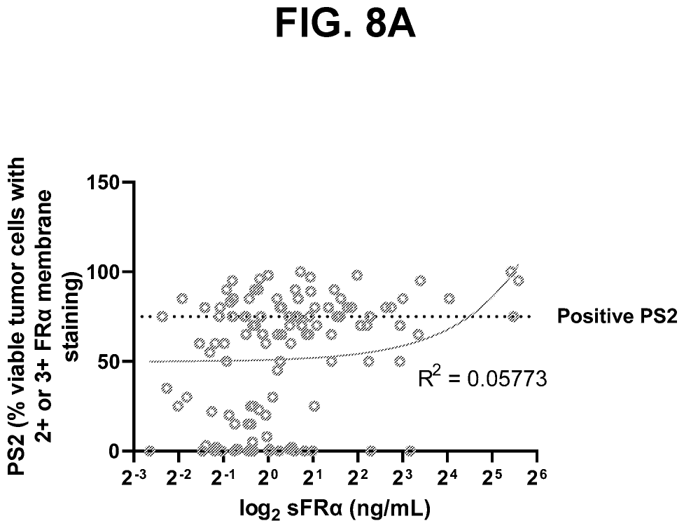

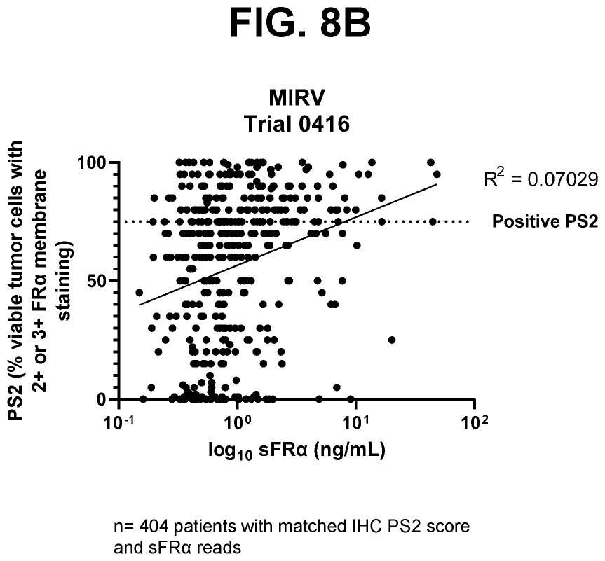

Soluble FRα data points from patients screened for the MIRASOL trial were plotted against the patient's corresponding IHC FRα PS2 score. No significant correlation between soluble FRα levels and surface tumor FRα expression (PS2) was observed (FIG. 8A), and these results were confirmed with a larger group of patients (FIG. 8B).

Thus, no significant correlations between soluble FRα and IHC scores were observed.

### Example 4. Soluble FRα Measurements Correlate Across Detection Methods

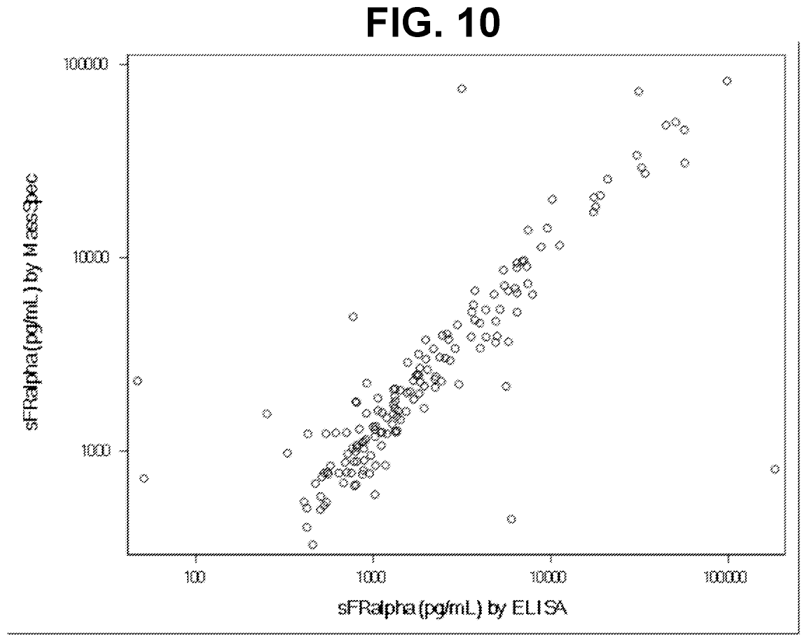

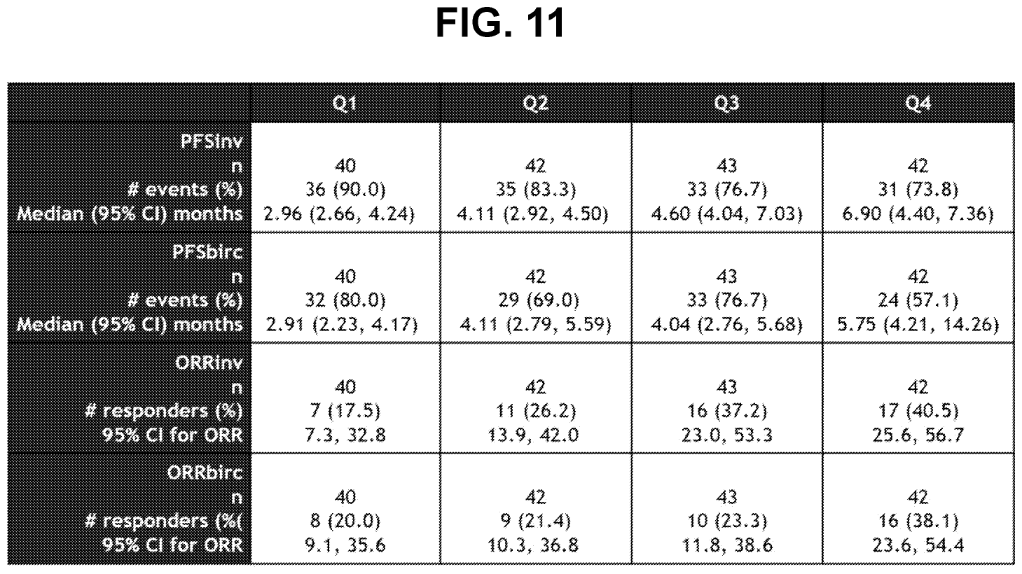

Soluble FRα levels were measured from patient serum from the FORWARD I trial using the Human FOLR1 Quantikine ELISA Kit (R&D Systems Cat #: DFLR10). All samples were collected prior to study drug dosing from patients who went on to receive mirvetuximab soravtansine as part of the I trial. The soluble FRα levels as measured by ELISA were assessed for correlation to solubleFRα levels as detected by liquid chromatography-mass spectrometry (LC-MS). The results, shown in FIG. 10, demonstrate that soluble FRα levels detected by ELISA and LC-MS correlate. In addition, PFS and ORR were compared in patients with different soluble FRα levels as measured by ELISA. The results, shown in FIG. 11, demonstrate increased PFS, and ORR in patients with increased soluble FRα levels as measured by ELISA.

It is to be appreciated that the Detailed Description section, and not the Summary and Abstract sections, is intended to be used to interpret the claims. The Summary and Abstract sections sets forth one or more, but not all, exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects provided herein will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects provided herein, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of treating cancer in a patient comprising administering a pharmaceutical composition comprising an anti-folate receptor α (FRα) active agent to a cancer patient with a soluble FRα level equal to or greater than a target soluble FRα level.

2. The method of claim 1, wherein the patient's soluble FRα has been detected in a sample obtained from the patient prior to the administration.

3. The method of claim 1 or 2, wherein a cancer sample obtained from the patient does not have a high FRα immunohistochemistry (IHC) score.

4. The method of claim 1 or 2, wherein a cancer sample obtained from the patient has a high FRα IHC score.

5. The method of claim 1 or 2, wherein no FRα IHC score has been obtained from the patient.

6. A method of treating cancer in a patient comprising (i) administering a pharmaceutical composition comprising an anti-FRα active agent to the patient if the patient has a soluble FRα level equal to or greater than a target soluble FRα level and/or a cancer sample obtained from the patient has a high FRα IHC score and (ii) administering chemotherapy to the patient if the patient does not have a soluble FRα level equal to or greater than a target soluble FRα level and a cancer sample obtained from the patient does not have a high FRα IHC score.

7. The method of claim any one of claims 1-6, further comprising determining the level of soluble FRα in sample obtained from the patient prior to the administering of the active agent.

8. The method of any one of claims 3, 4, 6, and 7, further comprising determining the FRα IHC score in a cancer sample obtained from the patient prior to the administration.

9. A method for identifying a cancer in a patient as likely to respond to an anti-FRα active agent, the method comprising assaying for soluble FRα in a sample obtained from the patient and optionally determining the FRα IHC score in a tumor sample obtained from the patient, wherein the presence of a soluble FRα level equal to or greater than a target soluble FRα level and/or a high FRα IHC score indicates the cancer is likely to respond to the anti-FRα active agent, optionally wherein the method further comprises administering a pharmaceutical composition comprising the anti-FRα active agent to the patient if the cancer is likely to respond.

10. The method of claim 10, wherein a level of soluble FRα that is not equal to or greater than a target soluble FRα level and an IHC score that is not high indicates the cancer is not likely to respond to a pharmaceutical composition comprising the anti-FRα active agent.

11. The method of claim 10, wherein a level of soluble FRα that is not equal to or greater than a target soluble FRα level and an IHC score that is not high indicates the cancer is likely to be more responsive to chemotherapy than to a pharmaceutical composition comprising the anti-FRα active agent.

12. The method of any one of claims 1-11, wherein the patient's level of soluble FRα is assessed using liquid chromatography-mass spectrometry (LC/MS), enzyme-linked immunosorbent assay (ELISA), and/or or meso scale discovery (MSD).

13. The method of any one of claims 1-12, wherein the target soluble FRα level is about 0.5 ng/mL.

14. The method of any one of claims 1-12, wherein the target soluble FRα level is about 0.6 ng/mL.

15. The method of any one of claims 1-12, wherein the target soluble FRα level is about 0.7 ng/mL.

16. The method of any one of claims 1-12, wherein the target soluble FRα level is about 0.75 ng/mL.

17. The method of any one of claims 1-12, wherein the target soluble FRα level is about 0.8 ng/mL.

18. The method of any one of claims 1-12, wherein the target soluble FRα level is about 0.9 ng/mL.

19. The method of any one of claims 1-12, wherein the target soluble FRα level is about 1 ng/mL.

20. The method of any one of claims 1-12, wherein the target soluble FRα level is about 1.1 ng/mL.

21. The method of any one of claims 1-12, wherein the target soluble FRα level is about 1.2 ng/mL.

22. The method of any one of claims 1-12, wherein the target soluble FRα level is about 1.25 ng/mL.

23. The method of any one of claims 1-12, wherein the target soluble FRα level is about 1.3 ng/mL.

24. The method of any one of claims 1-12, wherein the target soluble FRα level is about 1.4 ng/mL.

25. The method of any one of claims 1-12, wherein the target soluble FRα level is about 1.5 ng/mL.

26. The method of any one of claims 1-12, wherein the target soluble FRα level is about 1.6 ng/mL.

27. The method of any one of claims 1-12, wherein the target soluble FRα level is about 1.7 ng/mL.

28. The method of any one of claims 1-12, wherein the target soluble FRα level is about 1.75 ng/mL.

29. The method of any one of claims 1-12, wherein the target soluble FRα level is about 1.8 ng/mL.

30. The method of any one of claims 1-12, wherein the target soluble FRα level is about 1.9 ng/mL.

31. The method of any one of claims 1-12, wherein the target soluble FRα level is about 2.0 ng/mL.

32. The method of any one of claims 1-12, wherein the target soluble FRα level is about 2.1 ng/mL.

33. The method of any one of claims 1-12, wherein the target soluble FRα level is about 2.2 ng/mL.

34. The method of any one of claims 1-12, wherein the target soluble FRα level is about 2.25 ng/mL.

35. The method of any one of claims 1-12, wherein the target soluble FRα level is about 2.3 ng/mL.

36. The method of any one of claims 1-12, wherein the target soluble FRα level is about 2.4 ng/mL.

37. The method of any one of claims 1-12, wherein the target soluble FRα level is about 2.5 ng/mL.

38. The method of any one of claims 1-12, wherein the target soluble FRα level is about 2.6 ng/mL.

39. The method of any one of claims 1-12, wherein the target soluble FRα level is about 2.7 ng/mL.

40. The method of any one of claims 1-12, wherein the target soluble FRα level is about 2.75 ng/mL.

41. The method of any one of claims 1-12, wherein the target soluble FRα level is about 2.8 ng/mL.

42. The method of any one of claims 1-12, wherein the target soluble FRα level is about 2.9 ng/mL.

43. The method of any one of claims 1-12, wherein the target soluble FRα level is about 3.0 ng/mL.

44. The method of any one of claims 1-12, wherein the target soluble FRα level is about 3.1 ng/mL.

45. The method of any one of claims 1-12, wherein the target soluble FRα level is about 3.2 ng/mL.

46. The method of any one of claims 1-12, wherein the target soluble FRα level is about 3.25 ng/mL.

47. The method of any one of claims 1-12, wherein the target soluble FRα level is about 3.3 ng/mL.

48. The method of any one of claims 1-12, wherein the target soluble FRα level is about 3.4 ng/mL.

49. The method of any one of claims 1-12, wherein the target soluble FRα level is about 3.5 ng/mL.

50. The method of any one of claims 1-12, wherein the target soluble FRα level is about 3.6 ng/mL.

51. The method of any one of claims 1-12, wherein the target soluble FRα level is about 3.7 ng/mL.

52. The method of any one of claims 1-12, wherein the target soluble FRα level is about 3.75 ng/mL.

53. The method of any one of claims 1-12, wherein the target soluble FRα level is about 3.8 ng/mL.

54. The method of any one of claims 1-12, wherein the target soluble FRα level is about 3.9 ng/mL.

55. The method of any one of claims 1-12, wherein the target soluble FRα level is about 4.0 ng/mL.

56. The method of any one of claims 1-12, wherein the target soluble FRα level is about 4.1 ng/mL.

57. The method of any one of claims 1-12, wherein the target soluble FRα level is about 4.2 ng/mL.

58. The method of any one of claims 1-12, wherein the target soluble FRα level is about 4.25 ng/mL.

59. The method of any one of claims 1-12, wherein the target soluble FRα level is about 4.3 ng/mL.

60. The method of any one of claims 1-12, wherein the target soluble FRα level is about 4.4 ng/mL.

61. The method of any one of claims 1-12, wherein the target soluble FRα level is about 4.5 ng/mL.

62. The method of any one of claims 1-12, wherein the target soluble FRα level is about 4.6 ng/mL.

63. The method of any one of claims 1-12, wherein the target soluble FRα level is about 4.7 ng/mL.

64. The method of any one of claims 1-12, wherein the target soluble FRα level is about 4.75 ng/mL.

65. The method of any one of claims 1-12, wherein the target soluble FRα level is about 4.8 ng/mL.

66. The method of any one of claims 1-12, wherein the target soluble FRα level is about 4.9 ng/mL.

67. The method of any one of claims 1-12, wherein the target soluble FRα level is about 5 ng/mL.

68. The method of any one of claims 1-67, wherein the target soluble FRα level is the average soluble FRα level in patients with ovarian, primary peritonea, or fallopian tube cancer with a tumor with medium (50-74% cells positive) or high (at least 75% cells positive) membrane FRα levels as determined by percent staining (PS) 2+ staining intensity.

69. The method of any one of claims 3, 4, and 6-68, wherein a high IHC score refers to at least 75% of tumor cells with percent staining (PS) 2+ staining intensity.

70. The method of any one of claims 3, 4, and 6-68, wherein a high IHC score refers to at least 50% of tumor cells with PS2+ staining intensity.

71. The method of any one of claims 1-70, wherein the cancer is a solid tumor.

72. The method of any one of claims 1-71, wherein the cancer is selected from the group consisting of: ovarian cancer, uterine cancer, endometrial cancer, pancreatic cancer, renal cancer, lung cancer, peritoneal cancer, breast cancer, and fallopian tube cancer.

73. The method of claim 72, wherein the cancer is ovarian cancer, optionally wherein the ovarian cancer is platinum-resistant or platinum-refractory.

74. The method of claim 72, wherein the cancer is platinum-sensitive ovarian cancer.

75. The method of any one of claims 72-74, wherein the ovarian cancer is epithelial ovarian cancer.

76. The method of any one of claims 72, 73, or 75, wherein the cancer is platinum-resistant, advanced high-grade epithelial ovarian cancer.

77. The method of claim 72, wherein the cancer is uterine cancer.

78. The method of claim 72, wherein the cancer is endometrial cancer.

79. The method of claim 72, wherein the cancer is pancreatic cancer.

80. The method of claim 72, wherein the cancer is renal cancer.

81. The method of claim 72, wherein the cancer is lung cancer, optionally wherein the lung cancer is non-small cell lung cancer.

82. The method of claim 72, wherein the cancer is peritoneal cancer, optionally wherein the peritoneal cancer is primary peritoneal cancer.

83. The method of claim 72, wherein the cancer is breast cancer, optionally wherein the breast cancer is triple negative breast cancer (TNBC).

84. The method of claim 72, wherein the cancer is fallopian tube cancer.

85. The method of any one of claims 1-84, wherein the cancer is a recurrent cancer.

86. The method of any one of claims 1-85, wherein the active agent comprises an anti-FRα antibody or antigen-biding fragment thereof.

87. The method of claim 86, wherein the anti-FRα antibody or antigen-biding fragment thereof in the active agent binds to the same FRα epitope as an antibody comprising the VH of SEQ ID NO:38 and a VL of SEQ ID NO:44 and/or competitively inhibits binding of an antibody comprising the VH of SEQ ID NO:38 and a VL of SEQ ID NO:44 to FRα.

88. The method of claim 86 or 87, wherein the anti-FRα antibody or antigen-biding fragment thereof in the active agent comprises a variable heavy chain (VH) complementarity determining region (CDR) 1 comprising the amino acid sequence of SEQ ID NO:10, a VH-CDR2 comprising the amino acid sequence of SEQ ID NO:11, a VH-CDR3 comprising the amino acid sequence of SEQ ID NO:12, a variable light (VH)-CDR1 comprising the amino acid sequence of SEQ ID NO:15, a VL-CDR2 comprising the amino acid sequence of SEQ ID NO:16, and a VL-CDR3 comprising the amino acid sequence of SEQ ID NO: 17.

89. The method of any one of claims 86-88, wherein the anti-FRα antibody or antigen-biding fragment thereof in the active agent comprises a VH comprising the amino acid sequence of SEQ ID NO:38 and/or a VL comprising the amino acid sequence of SEQ ID NO:44.

90. The method of any one of claims 86-89, wherein the anti-FRα antibody or antigen-biding fragment thereof in the active agent comprises a heavy chain comprising the amino acid sequence of SEQ ID NO:50 and/or a light chain comprising the amino acid sequence of SEQ ID NO:56.

91. The method of any one of claims 86-89, wherein the anti-FRα antibody or antigen-biding fragment thereof in the active agent comprises a heavy chain comprising the same amino acid sequence as the amino acid sequence of the heavy chain encoded by the plasmid deposited with the American Type Culture Collection (ATCC) as PTA-10772 and/or a light chain comprising the same amino acid sequence as the amino acid sequence of the light chain encoded by the plasmid deposited with the ATCC as PTA-10774.

92. The method of any one of claims 86-91, wherein the anti-FRα antibody or antigen-biding fragment thereof in the active agent binds to the same FRα epitope as an antibody comprising the VH of SEQ ID NO:37 and a VL of SEQ ID NO:43 and/or competitively inhibits binding of an antibody comprising the VH of SEQ ID NO:37 and a VL of SEQ ID NO:43 to FRα.

93. The method of any one of claims 86-92, wherein the anti-FRα antibody or antigen-biding fragment thereof in the active agent comprises a variable heavy chain (VH) complementarity determining region (CDR) 1 comprising the amino acid sequence of SEQ ID NO:2, a VH-CDR2 comprising the amino acid sequence of SEQ ID NO:3, a VH-CDR3 comprising the amino acid sequence of SEQ ID NO:4, a variable light (VH)-CDR1 comprising the amino acid sequence of SEQ ID NO:7, a VL-CDR2 comprising the amino acid sequence of SEQ ID NO:8, and a VL-CDR3 comprising the amino acid sequence of SEQ ID NO:9.

94. The method of any one of claims 86-93, wherein the anti-FRα antibody or antigen-biding fragment thereof in the active agent comprises a VH comprising the amino acid sequence of SEQ ID NO:37 and/or a VL comprising the amino acid sequence of SEQ ID NO:43.

95. The method of any one of claims 86-94, wherein the anti-FRα antibody or antigen-biding fragment thereof in the active agent comprises a heavy chain comprising the amino acid sequence of SEQ ID NO:49 and/or a light chain comprising the amino acid sequence of SEQ ID NO:55.

96. The method of any one of claims 1-95, wherein the active agent comprises an antigen-biding fragment of an anti-FRα antibody.

97. The method of claim 96, wherein the antigen-binding fragment is a single-chain variable fragment (scFv), optionally wherein the scFV comprises the amino acid sequence of SEQ ID NO:60.

98. The method of any one of claims 1-97, wherein the anti-FRα antibody or antigen-biding fragment thereof in the active agent is a monospecific antibody or antigen-binding fragment thereof.

99. The method of any one of claims 1-97, wherein the anti-FRα antibody or antigen-biding fragment thereof in the active agent is a biparatopic antibody or antigen-binding fragment thereof.

100. The method of claim 99, wherein the biparatopic antibody or antigen-binding fragment thereof comprises the amino acid sequences of SEQ ID NOs:61, 62, and 56.

101. The method of claim 86, wherein the anti-FRα antibody or antigen-biding fragment thereof in the active agent binds to the same FRα epitope as an antibody comprising the VH of SEQ ID NO:99 and a VL of SEQ ID NO:101 and/or competitively inhibits binding of an antibody comprising the VH of SEQ ID NO:99 and a VL of SEQ ID NO:101 to FRα.

102. The method of claim 86 or 101, wherein the anti-FRα antibody or antigen-biding fragment thereof in the active agent comprises a variable heavy chain (VH) complementarity determining region (CDR) 1 comprising the amino acid sequence of SEQ ID NO:80 or 83, a VH-CDR2 comprising the amino acid sequence of SEQ ID NO:81 or 84, a VH-CDR3 comprising the amino acid sequence of SEQ ID NO:82 or 85, a variable light (VH)-CDR1 comprising the amino acid sequence of SEQ ID NO:86 or 89, a VL-CDR2 comprising the amino acid sequence of SEQ ID NO:87 or 90, and a VL-CDR3 comprising the amino acid sequence of SEQ ID NO:88.

103. The method of any one of claims 86, 101, or 102, wherein the anti-FRα antibody or antigen-biding fragment thereof in the active agent comprises a VH comprising the amino acid sequence of SEQ ID NO:99 and/or a VL comprising the amino acid sequence of SEQ ID NO:101.

104. The method of any one of claims 86 and 101-103, wherein the anti-FRα antibody or antigen-biding fragment thereof in the active agent comprises a heavy chain comprising the amino acid sequence of SEQ ID NO:103 and/or a light chain comprising the amino acid sequence of SEQ ID NO:104.

105. The method of claim 86, wherein the anti-FRα antibody or antigen-biding fragment thereof in the active agent binds to the same FRα epitope as an antibody comprising the VH of SEQ ID NO:100 and a VL of SEQ ID NO:102 and/or competitively inhibits binding of an antibody comprising the VH of SEQ ID NO:100 and a VL of SEQ ID NO:102 to FRα.

106. The method of claim 86 or 105, wherein the anti-FRα antibody or antigen-biding fragment thereof in the active agent comprises a variable heavy chain (VH) complementarity determining region (CDR) 1 comprising the amino acid sequence of SEQ ID NO:91 or 94, a VH-CDR2 comprising the amino acid sequence of SEQ ID NO:92 or 95, a VH-CDR3 comprising the amino acid sequence of SEQ ID NO:93, a variable light (VH)-CDR1 comprising the amino acid sequence of SEQ ID NO:96, a VL-CDR2 comprising the amino acid sequence of SEQ ID NO:97, and a VL-CDR3 comprising the amino acid sequence of SEQ ID NO:98.

107. The method of any one of claims 86, 105, or 106, wherein the anti-FRα antibody or antigen-biding fragment thereof in the active agent comprises a VH comprising the amino acid sequence of SEQ ID NO:100 and/or a VL comprising the amino acid sequence of SEQ ID NO:102.

108. The method of any one of claims 1-107 wherein the active agent is an immunoconjugate comprising an anti-FRα antibody or antigen-biding fragment thereof conjugated to a cytotoxic agent.

109. The method of claim 108, wherein the cytotoxic agent is conjugated to the anti-FRα antibody or antigen-biding fragment thereof by a linker, optionally wherein the linker is selected from the group consisting of a cleavable linker, a non-cleavable linker, a hydrophilic linker, and a dicarboxylic acid based linker.

110. The method of claim 109, wherein the linker is selected from the group consisting of N-(γmaleimidobutryloxy)sulfosuccinimide ester (sulfo-GMB S or sGMBS), 7 maleimidobutyric acid N-succinimidyl ester (GMBS), N-succinimidyl 4-(2-pyridyldithio)-2-sulfobutanoate (sulfo-SPDB); N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB), N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol] ester (NHS-PEG4-maleimide).

111. The method of claim 108 or 109, wherein the linker is sulfo-SPDB.

112. The method of claim 108 or 109, wherein the linker is sulfo-GMBS.

113. The method of claim 108 or 109, wherein the linker is GMBS.

114. The method of claim 108 or 109, wherein the linker is a cathepsin-B linker.

115. The method of any one of claims 108-114, wherein the cytotoxic agent is selected from the group consisting of a maytansinoid, maytansinoid analog, benzodiazepine, taxoid, CC-1065, CC-1065 analog, duocarmycin, duocarmycin analog, calicheamicin, dolastatin, dolastatin analog, auristatin, tomaymycin derivative, and leptomycin derivative or a prodrug of the agent.

116. The method of any one of claims 108-115, wherein the cytotoxic agent is a maytansinoid.

117. The method of claim 116, wherein the maytansinoid is DM4.

118. The method of claim 116, wherein the maytansinoid is DM21.

119. The method of any one of claims 108-114, wherein the cytotoxic agent is eribulin mesylate.

120. The method of any one of claims 108-114, wherein the cytotoxic agent is—aminophenyl hemiasterlin (SC209).

121. The method of any one of claims 108-120, wherein the immunoconjugate comprises 1 to 20 cytotoxic agents, optionally wherein the immunoconjugate comprises 1 to 10 cytotoxic agents.

122. The method of claim 108, wherein the immunoconjugate is represented by the following formula: 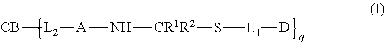
or a pharmaceutically acceptable salt thereof, wherein:
CB is an anti-FRα antibody or antigen-biding fragment thereof;
L₂ is represented by one of the following formula: 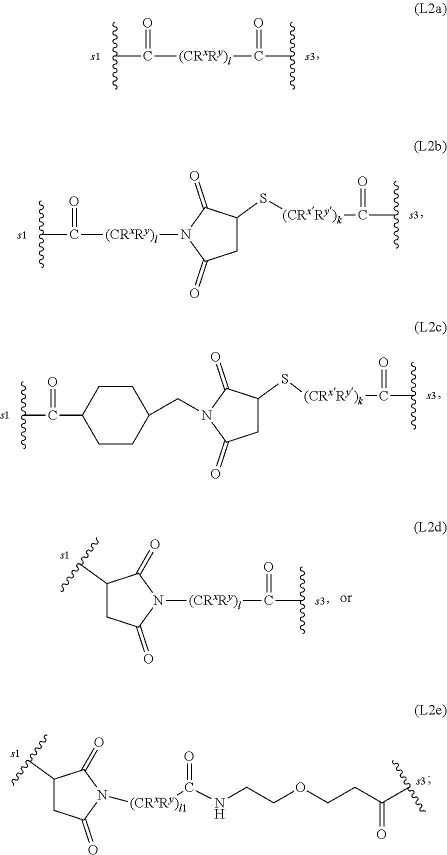
wherein:
R^{x}, R^{y}, R^{x′} and R^{y′}, for each occurrence, are independently H, —OH, halogen, —O—(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;
l1 is an integer from 2 to 5;
k1 is an integer from 1 to 5; and
s1 indicates the site connected to the cell-binding agent CB and s3 indicates the site connected to the A group;
A is an amino acid residue or a peptide comprising 2 to 20 amino acid residues;
R¹ and R² are each independently H or a C₁₋₃alkyl;
L₁ is represented by the following formula: 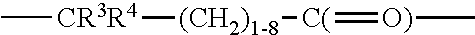
wherein R³ and R⁴ are each independently H or Me, and the —C(═O)— moiety in L₁ is connected to D;
D is represented by the following formula: 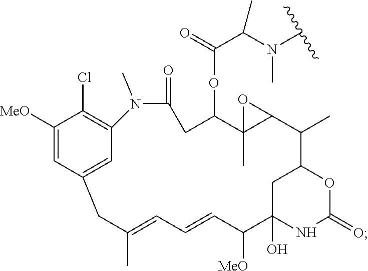 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]
q is an integer from 1 to 20.

123. The method of claim 122, wherein R^{x}, R^{y}, R^{x′} and R^{y′} are all H; and l and k are each independently an integer an integer from 2 to 6.

124. The method of claim 122 or 123, wherein A is a peptide containing 2 to 5 amino acid residues.

125. The method of claim 124, wherein A is selected from the group consisting of Gly-Gly-Gly, Ala-Val, Val-Ala, D-Val-Ala, Val-Cit, D-Val-Cit, Val-Lys, Phe-Lys, Lys-Lys, Ala-Lys, Phe-Cit, Leu-Cit, Ile-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Ala-Ala, D-Ala-Ala-Ala, Ala-D-Ala-Ala, Ala-Ala-D-Ala, Ala-Leu-Ala-Leu (SEQ ID NO:78), P-Ala-Leu-Ala-Leu (SEQ ID NO:79), Gly-Phe-Leu-Gly (SEQ ID NO:63), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Gln-Val, Asn-Ala, Gln-Phe, Gln-Ala, D-Ala-Pro, and D-Ala-tBu-Gly, wherein the first amino acid in each peptide is connected to L₂ group and the last amino acid in each peptide is connected to —NH—CR¹R²—S-L₁-D.

126. The method of any one of claims 122-125, wherein R¹ and R² are both H.

127. The method of any one of claims 122-125, wherein L₁ is —(CH₂)₄₋₆—C(═O)—.

128. The method of any one of claims 122-127, wherein D is represented by the following formula:

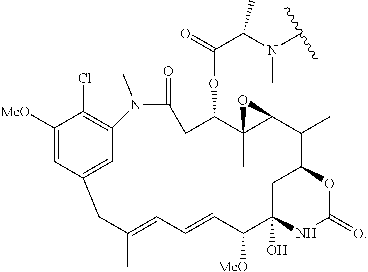 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)C(C)[C@@H]2O[C@@]12C]

129. The method of claim 108, wherein the immunoconjugate comprises an anti-FRα antibody or antigen-binding fragment thereof comprising a VH comprising the amino acid sequence of SEQ ID NO:38 and a VL comprising the amino acid sequence of SEQ ID NO:44, wherein the antibody or antigen-binding fragment thereof is conjugated to DM4 via a sulfo-SPDB linker.

130. The method of claim 129, wherein the immunoconjugate comprises an anti-FRα antibody comprising the a heavy chain comprising the amino acid sequence of SEQ ID NO:50 and a light chain comprising the amino acid sequence of SEQ ID NO:56.

131. The method of claim 129, wherein the pharmaceutical composition comprising an anti-folate receptor α (FRα) active agent comprises IMGN853.

132. The method of any one of claims 129-131, wherein the immunoconjugate is administered at a dose based on adjusted ideal body weight (AIBW).

133. The method of claim 132, wherein the immunoconjugate is administered at a dose of about 6 mg/kg AIBW.

134. The method of claim 132, wherein the immunoconjugate is administered at a dose of about 5 mg/kg AIBW.

135. The method of claim 108, wherein the immunoconjugate is represented by the following formula: 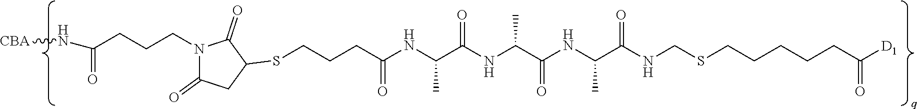
or a pharmaceutically acceptable salt thereof, wherein:
CBA is an antibody or an antigen-binding fragment comprising the amino acid sequences of SEQ ID NOs:61, 62, and 56;
D₁ is represented by the following formula: 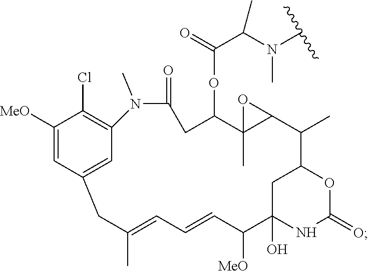 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]
and
q is an integer from 1 to 10.

136. The method of claim 101, wherein the pharmaceutical composition comprising an anti-folate receptor α (FRα) active agent comprises IMGN151.

137. The method of any one of claims 1-136, wherein the pharmaceutical composition comprises anti-FRα immunoconjugates comprising an average of 2 to 5 cytotoxic agents per antibody or antigen-binding fragment thereof, optionally wherein the anti-FRα immunoconjugates comprise an average of 3 to 4 cytotoxic agents per antibody or antigen-binding fragment thereof.

138. The method of claim 137, wherein the pharmaceutical composition comprises anti-FRα immunoconjugates comprising an average of 3.5 cytotoxic agents per antibody or antigen-binding fragment thereof.

139. The method of any one of claims 1-138, wherein soluble FRα is detected using a detection antibody or antigen-binding fragment thereof that specifically binds to FRα, wherein an antibody comprising a VH comprising the amino acid sequence of SEQ ID NO:38 and a VL comprising the amino acid sequence of SEQ ID NO:44 does not competitively inhibit binding of the detection antibody or antigen-binding fragment thereof to FRα.

140. The method of any one of claims 1-139, wherein soluble FRα is detected using a detection antibody or antigen-binding fragment thereof that specifically binds to FRα, wherein folic acid does not competitively inhibit binding of the detection antibody or antigen-binding fragment thereof to FRα.

141. The method of any one of claims 1-140, wherein soluble FRα is detected using a detection antibody or antigen-binding fragment thereof that specifically binds to FRα and comprises the VH-CDR1, VH-CDR2, VH-CDR3, VL-CDR1, VL-CDR2 and VL-CDR3 amino acid sequences of:
(a) SEQ ID NOs:18-20 and SEQ ID NOs:21-23, respectively; or
(b) SEQ ID NOs:24-26 and SEQ ID NOs:27-29, respectively.

142. The method of claim 141, wherein the detection antibody or antigen-binding fragment thereof comprises the VH and VL amino acid sequences of:
(a) SEQ ID NO:39 and SEQ ID NO:45, respectively; or
(b) SEQ ID NO:40 and SEQ ID NO:46, respectively.

143. The method of claim 142, wherein the detection antibody or antigen-binding fragment thereof comprises the heavy chain and light chain amino acid sequences of:
(a) SEQ ID NO:51 and SEQ ID NO:57, respectively; or
(b) SEQ ID NO:52 and SEQ ID NO:58, respectively.

144. The method of claim any one of claims 1-143, wherein soluble FRα is detected by (i) capturing FRα with an immunocapture reagent bound to a solid support (ii) eluting FRα from the solid support, (iii) digesting the eluted FRα, and (iv) performing LC/MS analysis on the digested FRα, wherein said FRα is detected by monitoring the chromatographic separation and mass spectrometric response of at least one signature FRα peptide.

145. The method of claim 144, wherein the solid support comprises a mass spectrometric immunoassay (MSIA) microcolumn.

146. The method of claim 144, wherein the solid support comprises magnetic beads.

147. The method of any one of claim 144-146, wherein at least one wash step is performed prior to eluting FRα from the solid support, optionally wherein two or more wash steps are performed prior to eluting FRα from the solid support.

148. The method of claim 147, where the wash step comprises contacting the FRα bound to the solid support with washing buffers, a salt solution, and a detergent.

149. The method of any one of claims 143-148, wherein FRα is eluted from the solid support with an acidic solution.

150. The method of claim 149, wherein the FRα is reduced and alkylated prior to digesting the FRα.

151. The method of any one of claims 143-149, wherein the FRα is digested with Trypsin/Lys-C.

152. The method of any one of claims 143-151, wherein digesting the FRα produces a peptide comprising the sequence of SEQ ID NO:68.

153. The method of any one of claims 143-152, wherein digesting the FRα produces a peptide comprising the sequence of SEQ ID NO:69.

154. The method of any one of claims 140-153, wherein digesting the FRα produces a peptide comprising the sequence of SEQ ID NO:70.

155. The method of any one of claims 140-154, wherein digesting the FRα produces a peptide comprising the sequence of SEQ ID NO:71.

156. The method of any one of claims 140-155, wherein at least two, at least three, or at least four signature peptides of FRα are selected and monitored at the LC/MS analysis step.

157. The method of claim 156, wherein the signature peptides comprise:
a. a peptide comprising the sequence of SEQ ID NO:68;
b. a peptide comprising the sequence of SEQ ID NO:69;
c. a peptide comprising the sequence of SEQ ID NO:70; and
d. a peptide comprising the sequence of SEQ ID NO:71.

158. The method of any one of claims 1-142, wherein the patient's level of soluble FRα is assessed using enzyme-linked immunosorbent assay (ELISA).

159. The method of any one of claims 1-158, wherein the soluble FRα is detected in a body fluid sample, optionally wherein the body fluid is plasma, serum, or ascites fluid.

160. The method of any one of claims 1-159, wherein the soluble FRα is detected in a peripheral blood sample.

161. The method of any one of claims 3, 4, or 6-160, wherein the FRα IHC score is obtained using IHC that distinguishes between staining intensity and staining uniformity in a tumor sample as compared to a reference sample.

162. The method of any one of claims 3, 4, or 6-161, wherein the FRα IHC score is obtained using an IHC antibody or antigen-binding fragment thereof that specifically binds to FRα and comprises the VH-CDR1, VH-CDR2, VH-CDR3, VL-CDR1, VL-CDR2 and VL-CDR3 amino acid sequences of: SEQ ID NOs:30-32 and SEQ ID NOs:33-35, respectively.

163. The method of claim 162, wherein the IHC detection antibody or antigen-binding fragment thereof comprises the VH and VL amino acid sequences of: SEQ ID NO:41 and SEQ ID NO:47, respectively.

164. The method of claim 163, wherein the detection antibody or antigen-binding fragment thereof comprises the heavy chain and light chain amino acid sequences of: SEQ ID NO:53 and SEQ ID NO 59, respectively.

165. A pharmaceutical composition for treating cancer in a patient with a soluble FRα level equal to or greater than a target soluble FRα according to the method of any one of claims 1-164, wherein the composition comprises an anti-folate receptor α (FRα) active agent.


SEQUENCE LISTING (104 sequences)

SEQ ID NO: 1 - protein, 257 aa, Artificial Sequence
  Full-length human FR-alpha
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 5 aa, Artificial Sequence
  VH-CDR1 FR57
SFGMH

SEQ ID NO: 3 - protein, 17 aa, Artificial Sequence
  VH-CDR2 FR57
YISSGSSTISYADSVKG

SEQ ID NO: 4 - protein, 9 aa, Artificial Sequence
  VH-CDR3 FR57
EAYGSSMEY

SEQ ID NO: 5 - protein, 10 aa, Artificial Sequence
  VH-CDR1 FR57
GFTFSSFGMH

SEQ ID NO: 6 - protein, 10 aa, Artificial Sequence
  VH-CDR2 FR57
YISSGSSTIS

SEQ ID NO: 7 - protein, 11 aa, Artificial Sequence
  VL-CDR1 FR57
RASQNINNNLH

SEQ ID NO: 8 - protein, 7 aa, Artificial Sequence
  VL-CDR2 FR57
YVSQSVS

SEQ ID NO: 9 - protein, 10 aa, Artificial Sequence
  VL-CDR3 FR57
QQSNSWPHYT

SEQ ID NO: 10 - protein, 5 aa, Artificial Sequence
  VH-CDR1 huMov19
GYFMN

SEQ ID NO: 11 - protein, 17 aa, Artificial Sequence
  VH-CDR2 huMov19
RIHPYDGDTFYNQKFQG

SEQ ID NO: 12 - protein, 9 aa, Artificial Sequence
  VH-CDR3 huMov19
YDGSRAMDY

SEQ ID NO: 13 - protein, 10 aa, Artificial Sequence
  VH-CDR1 huMov19
GYTFTGYFMN

SEQ ID NO: 14 - protein, 10 aa, Artificial Sequence
  VH-CDR2 huMov19
RIHPYDGDTF

SEQ ID NO: 15 - protein, 15 aa, Artificial Sequence
  VL-CDR1 huMov19
KASQSVSFAGTSLMH

SEQ ID NO: 16 - protein, 7 aa, Artificial Sequence
  VL-CDR2 huMov19
RASNLEA

SEQ ID NO: 17 - protein, 9 aa, Artificial Sequence
  VL-CDR3 huMov19
QQSREYPYT

SEQ ID NO: 18 - protein, 5 aa, Artificial Sequence
  VH-CDR1 muFR1-9
SFGMH

SEQ ID NO: 19 - protein, 17 aa, Artificial Sequence
  VH-CDR2 muFR1-9
YISSGSSTFYYADTVKG

SEQ ID NO: 20 - protein, 8 aa, Artificial Sequence
  VH-CDR3 muFR1-9
ELTGTFAY

SEQ ID NO: 21 - protein, 11 aa, Artificial Sequence
  VL-CDR1 muFR1-9
RASQSINNNLH

SEQ ID NO: 22 - protein, 7 aa, Artificial Sequence
  VL-CDR2 muFR1-9
YASQSIS

SEQ ID NO: 23 - protein, 10 aa, Artificial Sequence
  VL-CDR3 muFR1-9
QQSNSWPQVT

SEQ ID NO: 24 - protein, 5 aa, Artificial Sequence
  VH-CDR1 muFR1-13
RYSVH

SEQ ID NO: 25 - protein, 16 aa, Artificial Sequence
  VH-CDR2 muFR1-13
MIWSGGNTDYNSVFKS

SEQ ID NO: 26 - protein, 10 aa, Artificial Sequence
  VH-CDR3 muFR1-13
FDGKVSWFAY

SEQ ID NO: 27 - protein, 11 aa, Artificial Sequence
  VL-CDR1 muFR1-13
KASQSVSNDVL

SEQ ID NO: 28 - protein, 7 aa, Artificial Sequence
  VL-CDR2 muFR1-13
YAYNRYS

SEQ ID NO: 29 - protein, 9 aa, Artificial Sequence
  VL-CDR3 muFR1-13
QQDHSSPFT

SEQ ID NO: 30 - protein, 5 aa, Artificial Sequence
  VH-CDR1 muFRIHC2-1 (“2.1”)
NSYIH

SEQ ID NO: 31 - protein, 17 aa, Artificial Sequence
  VH-CDR2 muFRIHC2-1 (“2.1”)
WIYPESLNTQYNEKFKA

SEQ ID NO: 32 - protein, 13 aa, Artificial Sequence
  VH-CDR3 muFRIHC2-1 (“2.1”)
RGIYYYSPYALDH

SEQ ID NO: 33 - protein, 16 aa, Artificial Sequence
  VL-CDR1 muFRIHC2-1 (“2.1”)
KSSKSLLNSDGFTYLD

SEQ ID NO: 34 - protein, 7 aa, Artificial Sequence
  VL-CDR2 muFRIHC2-1 (“2.1”)
LVSNHFS

SEQ ID NO: 35 - protein, 9 aa, Artificial Sequence
  VL-CDR3 muFRIHC2-1 (“2.1”)
FQSNYLPLT

SEQ ID NO: 36 - protein, 118 aa, Artificial Sequence
  Heavy Chain Variable (VH) Sequence - FR57
EVQLVESGGGLVQPGGSRRLSCAASGFTFSSFGMHWVRQAPGKGLEWVAYISSGSSTISYADSVKGRFTISRDNSKKTLLLQMTSLRAEDTAMYYCAREAYGSSMEYWGQGTLVTVSS

SEQ ID NO: 37 - protein, 118 aa, Artificial Sequence
  Heavy Chain Variable (VH) Sequence - FR57 E6Q; G44C
EVQLVQSGGGLVQPGGSRRLSCAASGFTFSSFGMHWVRQAPGKCLEWVAYISSGSSTISYADSVKGRFTISRDNSKKTLLLQMTSLRAEDTAMYYCAREAYGSSMEYWGQGTLVTVSS

SEQ ID NO: 38 - protein, 118 aa, Artificial Sequence
  Heavy Chain Variable (VH) Sequence - huMov19
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 39 - protein, 117 aa, Artificial Sequence
  Heavy Chain Variable (VH) Sequence - muFR1-9
QVQLVESGGGLVQPGGSRKLSCAASGFTFSSFGMHWVRQAPEKGLEWVAYISSGSSTFYYADTVKGRFTISRDNPKNTLFLQMTSLRSEDTAMYYCAKELTGTFAYWGQGTLVTVSA

SEQ ID NO: 40 - protein, 118 aa, Artificial Sequence
  Heavy Chain Variable (VH) Sequence - muFR1-13
QVQLKESGPDLVAPSQSLSITCTVSGFSLSRYSVHWIRQPPGKGLEWLGMIWSGGNTDYNSVFKSRLNITKDNSKSQVFLKMNSLQTDDTAIYYCATFDGKVSWFAYWGQGTLVTVSA

SEQ ID NO: 41 - protein, 122 aa, Artificial Sequence
  Heavy Chain Variable (VH) Sequence - muFRIHC2-1 (“2.1”)
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSS

SEQ ID NO: 42 - protein, 108 aa, Artificial Sequence
  Light Chain Variable (VL) Sequence - FR57
EIVLTQSPATLSVTPGDRVSLSCRASQNINNNLHWYQQKPGQSPRLLIKYVSQSVSGIPDRFSGSGSGTDFTLSISSVEPEDFGMYFCQQSNSWPHYTFGQGTKLEIK

SEQ ID NO: 43 - protein, 108 aa, Artificial Sequence
  Light Chain Variable (VL) Sequence - FR57 F83E; Q101C
EIVLTQSPATLSVTPGDRVSLSCRASQNINNNLHWYQQKPGQSPRLLIKYVSQSVSGIPDRFSGSGSGTDFTLSISSVEPEDEGMYFCQQSNSWPHYTFGCGTKLEIK

SEQ ID NO: 44 - protein, 111 aa, Artificial Sequence
  Light Chain Variable (VL) Sequence - huMov19
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 45 - protein, 109 aa, Artificial Sequence
  Light Chain Variable (VL) Sequence - muFR1-9
DIVLTQSPATLSVTPGDSVSLSCRASQSINNNLHWYQQKSHESPRLLIKYASQSISGIPSRFSGSGSGTDFTLSINSVETEDFGMYFCQQSNSWPQVTFGAGTKLELKR

SEQ ID NO: 46 - protein, 108 aa, Artificial Sequence
  Light Chain Variable (VL) Sequence - muFR1-13
SIVMTQTPKFLLVSTGDRFTITCKASQSVSNDVLWYQQKPGQSPKLLIYYAYNRYSGVPDRFTGSGYGTDFTFTITTVQSEDLAVYFCQQDHSSPFTFGSGTKLEIKR

SEQ ID NO: 47 - protein, 114 aa, Artificial Sequence
  Light Chain Variable (VL) Sequence - muFRIHC2-1 (“2.1”)
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKR

SEQ ID NO: 48 - protein, 447 aa, Artificial Sequence
  Full-Length Heavy Chain Amino Acid Sequence - FR57
EVQLVESGGGLVQPGGSRRLSCAASGFTFSSFGMHWVRQAPGKGLEWVAYISSGSSTISYADSVKGRFTISRDNSKKTLLLQMTSLRAEDTAMYYCAREAYGSSMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 447 aa, Artificial Sequence
  Full-Length Heavy Chain Amino Acid Sequence - FR57 E6Q; G44C
EVQLVQSGGGLVQPGGSRRLSCAASGFTFSSFGMHWVRQAPGKCLEWVAYISSGSSTISYADSVKGRFTISRDNSKKTLLLQMTSLRAEDTAMYYCAREAYGSSMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 447 aa, Artificial Sequence
  Full-Length Heavy Chain Amino Acid Sequence - huMov19
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 441 aa, Artificial Sequence
  Full-Length Heavy Chain Amino Acid Sequence - muFR1-9
QVQLVESGGGLVQPGGSRKLSCAASGFTFSSFGMHWVRQAPEKGLEWVAYISSGSSTFYYADTVKGRFTISRDNPKNTLFLQMTSLRSEDTAMYYCAKELTGTFAYWGQGTLVTVSAAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 52 - protein, 454 aa, Artificial Sequence
  Full-Length Heavy Chain Amino Acid Sequence - muFR1-13
QVQLKESGPDLVAPSQSLSITCTVSGFSLSRYSVHWIRQPPGKGLEWLGMIWSGGNTDYNSVFKSRLNITKDNSKSQVFLKMNSLQTDDTAIYYCATFDGKVSWFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 53 - protein, 446 aa, Artificial Sequence
  Full-Length Heavy Chain Amino Acid Sequence - muFRIHC2-1 (“2.1”)
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSSAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 54 - protein, 215 aa, Artificial Sequence
  Full-Length Light Chain Amino Acid Sequence - FR57
EIVLTQSPATLSVTPGDRVSLSCRASQNINNNLHWYQQKPGQSPRLLIKYVSQSVSGIPDRFSGSGSGTDFTLSISSVEPEDFGMYFCQQSNSWPHYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 215 aa, Artificial Sequence
  Full-Length Light Chain Amino Acid Sequence - FR57 F83E; Q101C
EIVLTQSPATLSVTPGDRVSLSCRASQNINNNLHWYQQKPGQSPRLLIKYVSQSVSGIPDRFSGSGSGTDFTLSISSVEPEDEGMYFCQQSNSWPHYTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 218 aa, Artificial Sequence
  Full-Length Light Chain Amino Acid Sequence - huMov19
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 215 aa, Artificial Sequence
  Full-Length Light Chain Amino Acid Sequence - muFR1-9
DIVLTQSPATLSVTPGDSVSLSCRASQSINNNLHWYQQKSHESPRLLIKYASQSISGIPSRFSGSGSGTDFTLSINSVETEDFGMYFCQQSNSWPQVTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 214 aa, Artificial Sequence
  Full-Length Light Chain Amino Acid Sequence - muFR1-13
SIVMTQTPKFLLVSTGDRFTITCKASQSVSNDVLWYQQKPGQSPKLLIYYAYNRYSGVPDRFTGSGYGTDFTFTITTVQSEDLAVYFCQQDHSSPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 59 - protein, 220 aa, Artificial Sequence
  Full-Length Light Chain Amino Acid Sequence - muFRIHC2-1 (“2.1”)
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 60 - protein, 246 aa, Artificial Sequence
  FR57scFv - scFv in VL (F83E; Q101C)-(G4S)4-VH (E6Q; G44C) orientation
EIVLTQSPATLSVTPGDRVSLSCRASQNINNNLHWYQQKPGQSPRLLIKYVSQSVSGIPDRFSGSGSGTDFTLSISSVEPEDEGMYFCQQSNSWPHYTFGCGTKLEIKGGGGSGGGGSGGGGSGGGGSEVQLVQSGGGLVQPGGSRRLSCAASGFTFSSFGMHWVRQAPGKCLEWVAYISSGSSTISYADSVKGRFTISRDNSKKTLLLQMTSLRAEDTAMYYCAREAYGSSMEYWGQGTLVTVSS

SEQ ID NO: 61 - protein, 479 aa, Artificial Sequence
  FR57scFv-Fc-knob (C220S, T366W)
EIVLTQSPATLSVTPGDRVSLSCRASQNINNNLHWYQQKPGQSPRLLIKYVSQSVSGIPDRFSGSGSGTDFTLSISSVEPEDEGMYFCQQSNSWPHYTFGCGTKLEIKGGGGSGGGGSGGGGSGGGGSEVQLVQSGGGLVQPGGSRRLSCAASGFTFSSFGMHWVRQAPGKCLEWVAYISSGSSTISYADSVKGRFTISRDNSKKTLLLQMTSLRAEDTAMYYCAREAYGSSMEYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 447 aa, Artificial Sequence
  Mov19-Fc-hole (T366S, L368A, Y407V)
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 4 aa, Artificial Sequence
  Synthetic sequence
GFLG

SEQ ID NO: 64 - protein, 4 aa, Artificial Sequence
  Peptide
GGGS

SEQ ID NO: 65 - protein, 5 aa, Artificial Sequence
  Peptide
GGGGS

SEQ ID NO: 66 - protein, 15 aa, Artificial Sequence
  Linker
GGGGSGGGGSGGGGS

SEQ ID NO: 67 - protein, 20 aa, Artificial Sequence
  Linker
GGGGSGGGGSGGGGSGGGGS

SEQ ID NO: 68 - protein, 11 aa, Artificial Sequence
  sFR-alpha Signature Peptide Sequence 1
TELLNVCMNAK

SEQ ID NO: 69 - protein, 5 aa, Artificial Sequence
  sFR-alpha Signature Peptide Sequence 2
IAWAR

SEQ ID NO: 70 - protein, 8 aa, Artificial Sequence
  sFR-alpha Signature Peptide Sequence 3
VLNVPLCK

SEQ ID NO: 71 - protein, 19 aa, Artificial Sequence
  sFR-alpha Signature Peptide Sequence 4
CIQMWFDPAQGNPNEEVAR

SEQ ID NO: 72 - protein, 216 aa, Artificial Sequence
  Exemplary IgG1 Fc Region
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 215 aa, Artificial Sequence
  Exemplary IgG2 Fc Region
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 74 - protein, 216 aa, Artificial Sequence
  Exemplary IgG4 Fc Region
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 75 - protein, 216 aa, Artificial Sequence
  Exemplary L234A/L235A IgG1 Fc Region
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 216 aa, Artificial Sequence
  Exemplary N297A IgG1 Fc Region
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 216 aa, Artificial Sequence
  Exemplary N297Q IgG1 Fc Region
APELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 4 aa, Artificial Sequence
  Synthetic sequence
ALAL

SEQ ID NO: 79 - protein, 4 aa, Artificial Sequence
  Synthetic sequence
  MOD_RES (1)..(1): Beta-Ala
ALAL

SEQ ID NO: 80 - protein, 5 aa, Artificial Sequence
  VH-CDR1 MORAb-003
GYGLS

SEQ ID NO: 81 - protein, 17 aa, Artificial Sequence
  VH-CDR2 MORAb-003
MISSGGSYTYYADSVKG

SEQ ID NO: 82 - protein, 10 aa, Artificial Sequence
  VH-CDR3 MORAb-003
HGDDPAWFAY

SEQ ID NO: 83 - protein, 8 aa, Artificial Sequence
  VH-CDR1 MORAb-003
GFTFSGYG

SEQ ID NO: 84 - protein, 8 aa, Artificial Sequence
  VH-CDR2 MORAb-003
ISSGGSYT

SEQ ID NO: 85 - protein, 12 aa, Artificial Sequence
  VH-CDR3 MORAb-003
ARHGDDPAWFAY

SEQ ID NO: 86 - protein, 12 aa, Artificial Sequence
  VL-CDR1 MORAb-003
SVSSSISSNNLH

SEQ ID NO: 87 - protein, 7 aa, Artificial Sequence
  VL-CDR2 MORAb-003
GTSNLAS

SEQ ID NO: 88 - protein, 11 aa, Artificial Sequence
  VL-CDR3 MORAb-003
QQWSSYPYMYT

SEQ ID NO: 89 - protein, 7 aa, Artificial Sequence
  VL-CDR1 MORAb-003
SSISSNN

SEQ ID NO: 90 - protein, 3 aa, Artificial Sequence
  VL-CDR2 MORAb-003
GTS

SEQ ID NO: 91 - protein, 5 aa, Artificial Sequence
  VH-CDR1 1848-H01
TQSIH

SEQ ID NO: 92 - protein, 17 aa, Artificial Sequence
  VH-CDR2 1848-H01
DIFPIDGITDYADSVKG

SEQ ID NO: 93 - protein, 15 aa, Artificial Sequence
  VH-CDR3 1848-H01
GSWSWPSGMDYYLDY

SEQ ID NO: 94 - protein, 7 aa, Artificial Sequence
  VH-CDR1 1848-H01
GFNIRTQ

SEQ ID NO: 95 - protein, 6 aa, Artificial Sequence
  VH-CDR2 1848-H01
FPIDGI

SEQ ID NO: 96 - protein, 11 aa, Artificial Sequence
  VL-CDR1 1848-H01
RASQDVNTAVA

SEQ ID NO: 97 - protein, 7 aa, Artificial Sequence
  VL-CDR2 1848-H01
SASFLYS

SEQ ID NO: 98 - protein, 9 aa, Artificial Sequence
  VL-CDR3 1848-H01
QQHYTTPPT

SEQ ID NO: 99 - protein, 119 aa, Artificial Sequence
  Heavy Chain Variable (VH) Sequence - MORAb-003
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 100 - protein, 124 aa, Artificial Sequence
  Heavy Chain Variable (VH) Sequence - 1848-H01
EVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSS

SEQ ID NO: 101 - protein, 110 aa, Artificial Sequence
  Light Chain Variable (VL) Sequence - MORAb-003
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 102 - protein, 107 aa, Artificial Sequence [sequence omitted]

SEQ ID NO: 103 - protein, 449 aa, Artificial Sequence
  Full-Length Heavy Chain Amino Acid Sequence - MORAb-003
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSISSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 217 aa, Artificial Sequence
  Full-Length Light Chain Amino Acid Sequence - MORAb-003
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC